(19) United States
(12) Patent Application Publication
(10) Pub. No.: US 2012/0266262 A1
(43) Pub. Date: Oct. 18, 2012

(54) METHODS AND COMPOSITIONS RELATING TO ZPA POLYPEPTIDES

(76) Inventors: Avi J. Ashkenazi, San Mateo, CA (US); Reece Hart, San Francisco, CA (US); Erica Kratz, San Mateo, CA (US); Kiran Mukhyala, Belmont, CA (US)

(21) Appl. No.: 13/455,909

(22) Filed: Apr. 25, 2012

Related U.S. Application Data
(63) Continuation of application No. 12/299,449, filed on Sep. 23, 2009, now abandoned.
(60) Provisional application No. 60/797,703, filed on May 4, 2006.

(51) Int. Cl. C07K 14/435; A61K 38/17; A61K 39/395; A61P 25/00; A61P 35/00; C12Q 1/68; A61P 37/00; A61P 7/00; A01K 67/027; G01N 33/566; C12Q 1/18; C12Q 1/37; C12N 15/12; A61P 31/12

(52) U.S. Cl. 800/9; 530/350; 536/23.5; 514/18.9; 424/139.1; 514/17.7; 436/501; 435/32; 435/23; 435/6.13

(57) ABSTRACT

The present invention provides ZPA polypeptides, antibodies, nucleic acid molecules, antagonists, agonists, potentiators and compositions relating to ZPA polypeptides, and methods of identifying, making and using the same, that are useful for treating and preventing diseases and for medical diagnosis and research. The present invention also provides model systems for the intrinsic apoptotic pathway.

### FIELD OF THE INVENTION

The present invention is directed to ZPA polypeptides, antibodies, nucleic acid molecules, antagonists, agonists, and compositions relating to ZPA polypeptides, and methods of making and using the same, including methods for diagnosing and treating of apoptosis-related disorders in mammals. The present invention is also directed to model systems for the intrinsic apoptotic pathway.

### BACKGROUND OF THE INVENTION

Uncontrolled cell growth is the cause of many illnesses in a variety of cell types. For example, cancer occurs when there is an increase in the number of abnormal, or neoplastic, cells derived from a normal tissue that proliferate to form a tumor mass. The tumor cells often invade the adjacent tissues and can spread via the blood or lymphatic system to regional lymph nodes and to distant sites via a process called metastasis. In a cancerous growth, a cell proliferates under conditions in which normal cells would not grow. Cancer manifests itself in a wide variety of forms, characterized by different degrees of invasiveness and aggressiveness. Malignant tumors (cancers) are the second leading cause of death in the United States, after heart disease (Boring et al., CA Cancel J. Clin. 43:7 (1993)).

Much research has been devoted to discovering new treatments for cell proliferative disorders, such as cancer. Despite recent advances, there is a great need to identify and understand the role of new cellular targets for modulating cell proliferation and to develop alternative or more effective methods of treatment and therapeutic and diagnostic agents. There is also a need to develop alternative therapeutics and methods for treating specific cell types and for treating illnesses caused by or associated with abnormal cell proliferation, such as cancers. One approach to developing anti-cancer therapeutics is to study the mechanisms of apoptosis, also known as programmed cell death.

Robust control of the apoptotic mechanisms that determine cell fate is required for organism development, homeostasis, and cellular damage response. Dysregulation of such pathways often leads to serious diseases. For example, many cancers selectively inhibit pro-apoptotic pathways and/or enhance pro-survival pathways in order to evade host responses intended to regulate growth (Kirkin et al., Biochim Biophys Acta 1644 (2-3): 229-249 (2004); LeBlanc et al., Nature Med. 8:2 274-281 (2002); Cory and Adams, Trends Biochem. Sci. 26: 61-66 (2001)). Therefore, understanding and treating a variety of diseases, including cancer, autoimmune diseases, and degenerative disorders necessitates an understanding of apoptosis (Strasser, Nat. Rev. Immunol. 5: 189-200 (2005); Strasser et al., Biochim Biophys Acta 1333: F151-178 (1997)).

Two apoptosis signaling pathways have been described in mammals (Anderson et al., Nat. Rev. Drug Discov. 4(5): 399-409 (2005)): the extrinsic pathway, typically initiated by the death-inducing ligands of the TNF family, and the intrinsic pathway, primarily responding to intracellular stimuli mediated by the Bcl-2 family (Strasser et al., Nat. Rev. 1 mm. 5:189-200 (2005); Borner, Mol. Immunol. 39: 615-647 (2003)), but also activated by components of the extrinsic pathway.

The Bcl-2 proteins are characterized by four distinct alpha-helical sequence motifs known as the Bcl-2 homology (BH) domains BH1 to BH4. In some cases, Bcl-2 proteins also have a C-terminal transmembrane region that localizes them to the cytoplasmic face of the outer mitochondrial membrane, nuclear envelope, or endoplasmic reticulum (Bonier, Mol. Immunol. 39: 615-647 (2003)). Bcl-2 protein family members may be divided into three groups: (i) pro-survival Bcl-2 like proteins (e.g., Bcl-2, Bcl-x_{L}, Bcl-w, Mcl-1, A1/Bfl-1, NR-13, BHRF1, LMW5-HL, ORF16, v-Bcl-2(KSHV), E1B-19K, CED-9, Boo/DIVA/Bcl2-L-10, and Bcl-B); (ii) pro-apoptotic multidomain proteins (e.g., Bax, Bak, Bok/Mtd, and Bcl-x_{S}); and (iii) BH3-only pro-apoptotic proteins (e.g., Bik/Nbk, Blk, Hrk/DP5, BNip3, BimL/Bod, Bad, Bid, EGL-1, Noxa, Puma/Bbc3, and Bmf) (id.). Most pro-survival members contain all four BH domains while most multi-domain pro-apoptotic proteins lack a BH4 domain.

In unstimulated cells, interactions between pro-survival and pro-apoptotic multidomain family members prevent the Bax-like proteins from oligomerizing at the mitochondrial membrane and initiating the apoptotic program. Upon stimulation, BH3-only proteins relieve the inhibition of Bax-like proteins by dimerizing with the pro-survival proteins, freeing the pro-apoptotic multidomain proteins to compromise mitochondrial membrane potential and initiate apoptosis. Previous experiments have demonstrated that several members of the Bcl-2 family are critical for normal development (Lindsten et al., Mol. Cell. 6: 1389-1399 (2000); Motoyama et al., Science 267: 1506-1510 (1995); Veis et al., Cell 75: 229-240 (1993); Rinkenberger et al., Genes Dev. 14: 23-27 (2000)). However, the function of this protein family during development is largely unknown: even in Bcl-2-related gene knockouts with significant developmental effects, neither the initiating apoptotic signal nor the BH3-only proteins activated in response to the signal are known.

A number of novel Bcl-2 family members have been identified in vertebrates by sequence similarity to Bcl-2, the eponymous member of the family named for its role in B cell lymphoma (Tsujimoto et al., Science 228: 1440-1443 (1985)). However, because Bcl-2 family members are critical to development and regulation, aberrations in or deletions of one or more members of this family of proteins often cause pathologies which prevent a characterization of their functional importance, or result in nonviable animals in the first instance. A model system in which developmental and regulatory changes could be monitored from the earliest stages of growth would provide a crucial tool for addressing questions regarding the roles of the Bcl-2 family of genes in apoptosis.

Zebrafish (Danio rerio) have served as a useful model system for a variety of biological pathways. Zebrafish can serve as an exceptional model for studying apoptosis not only because development in the fish is rapid, and zebrafish embryos remain transparent throughout most of embryogenesis, but also because of the availability of mutant zebrafish lines displaying abnormal apoptosis (see, e.g., Cole and Ross, Devel. Biol. 240: 123-142 (2001)). Apoptosis patterns have been examined in zebrafish, so detection of apoptotic cells and the general dynamics of apoptosis are known in that organism (id.). However, the biochemical pathways responsible for those apoptotic patterns in zebrafish have not been characterized. It remains an open question whether the intrinsic apoptotic pathway functions in the zebrafish.

A prerequisite to establishing zebrafish as a model for apoptotic signaling through the intrinsic pathway is a demonstration that the major members of the Bcl-2 family are present in the zebrafish. Several studies have tried to identify Bcl-2 family members in zebrafish. Inohara and Nunez found many zebrafish genes homologous to mammalian and avian extrinsic pathway members such as the caspases, but only identified eight zebrafish genes putatively related to only six members of the intrinsic pathway Bcl-2 family (Bcl-x_{L}, Mcl-1, NR-13, Bax, BNIP3, and Bad) (Inohara and Nunez, Cell Death Diff. 7: 509-510 (2000)). Coultas et al. exhaustively searched the zebrafish non-redundant and EST Genbank databases by tblastn and identified only three further BH3-only Bcl-2 family members: Bid, Noxa, and Bmf (Cell Death Differ. 9: 1163-1166 (2002)). In fact, that group particularly commented on the failure to identify Bik, Bim, and Puma in zebrafish using translated BLAST searching (id.). This observation was recently confirmed by Aouacheria et al. after exhaustively searching Ensembl and GenBank nucleotide and protein sequences using PSI-BLAST and tblastn (Mol. Biol. Evol. 22(12): 2395-416 (2005)).

### SUMMARY OF THE INVENTION

The present invention provides new model systems for investigating apoptosis in vivo and in vitro, and provides methods for identifying agents that modulate apoptosis. The present invention also provides new therapeutic agents, diagnostic agents, and methods for treating or preventing apoptosis-related disease, including cancer, by targeting apoptosis, particularly the intrinsic apoptotic pathway.

In certain embodiments, the invention provides zebrafish pro-apoptosis (“ZPA”) polypeptides and polynucleotides. In one embodiment, a polypeptide having an amino acid sequence selected from SEQ ID NOs: 1, 5, 7, and 9 is provided, wherein the polypeptide is a zebrafish Bcl-2-related (“B2R”) pro-apoptotic polypeptide. In another embodiment, a polypeptide having an amino acid sequence of SEQ ID NO: 1 is provided, wherein the polypeptide is a zebrafish B2R multidomain pro-apoptotic polypeptide. In another embodiment, a polypeptide having an amino acid selected from SEQ ID NOs: 5, 7, and 9 is provided, wherein the polypeptide is a zebrafish B2R BH3-only pro-apoptotic polypeptide. In another embodiment, a polynucleotide having a nucleotide sequence selected from SEQ ID NOs: 2, 6, 8, and 10 is provided, wherein the polynucleotide encodes a zebrafish B2R pro-apoptotic polypeptide. In another embodiment, a polynucleotide having a nucleotide sequence of SEQ ID NO: 1 is provided, wherein the polynucleotide encodes a zebrafish B2R multidomain pro-apoptotic polypeptide. In another embodiment, a polynucleotide having a nucleotide sequence selected from SEQ ID NOs: 6, 8, and 10 is provided, wherein the polynucleotide encodes a zebrafish B2R BH3-only pro-apoptotic polypeptide.

In other embodiments, the invention provides zebrafish transgenic for one or more apoptosis-related proteins. In one embodiment, a transgenic zebrafish is provided, wherein one or more polynucleotides selected from SEQ ID NOs: 2, 6, 8, and 10 is deleted. In another embodiment, a transgenic zebrafish is provided, wherein the expression of one or more polynucleotides selected from SEQ ID NOs: 2, 6, 8, and 10 is modulated relative to the expression of the one or more polynucleotides in a wild-type zebrafish. In one aspect, the expression is increased. In another aspect, the expression is decreased. In another embodiment, a transgenic zebrafish is provided, wherein one or more polypeptides selected from SEQ ID NOs: 1, 5, 7, and 9 are not expressed. In another embodiment, a transgenic zebrafish is provided, wherein the expression of one or more polypeptides selected from SEQ ID NOs: 1, 5, 7, and 9 is modulated relative to the expression of the one or more polypeptides in a wild-type zebrafish. In one aspect, the expression is increased. In another aspect, the expression is decreased.

In another embodiment, a transgenic zebrafish is provided, wherein one or more endogenous B2R genes are replaced with a B2R gene counterpart from another organism. In one aspect, the counterpart is mammalian. In another aspect, the counterpart is human. In another aspect, all of the endogenous B2R genes are replaced with B2R gene counterparts from another organism. In one aspect, the counterpart is mammalian. In another aspect, the counterpart is human. In another embodiment, a transgenic zebrafish is provided, wherein one or more endogenous intrinsic apoptotic pathway genes are replaced with an intrinsic apoptotic pathway gene counterpart from another organism. In one aspect, the counterpart is mammalian. In another aspect, the counterpart is human. In another aspect, the one or more endogenous intrinsic apoptotic pathway genes are selected from SEQ ID NOs: 2, 6, 8, and 10.

In another embodiment, a transgenic zebrafish is provided, wherein all of the endogenous intrinsic apoptotic pathway genes are replaced with intrinsic apoptotic pathway gene counterparts from another organism. In one aspect, the counterpart is mammalian. In another aspect, the counterpart is human. In another aspect, the endogenous intrinsic apoptotic pathway genes include SEQ ID NOs: 2, 6, 8, and 10. In certain embodiments, the invention provides model systems for apoptosis. In one embodiment, a model system for apoptosis is provided comprising a zebrafish as described in any of the previous embodiments. In one aspect, the model system is a model system for the intrinsic apoptotic pathway. In another embodiment, an in vitro model system for apoptosis is provided comprising at least one polypeptide encoded by an amino acid sequence selected from SEQ ID NOs: 1, 5, 7, and 9. In one aspect, the model system is a model system for the intrinsic apoptotic pathway. In another aspect, the model system is a model system for the extrinsic apoptotic pathway. In another embodiment, an in vitro model system for apoptosis is provided comprising at least one polynucleotide encoded by a nucleotide sequence selected from SEQ ID NOs: 2, 6, 8, and 10. In one aspect, the model system is a model system for the intrinsic apoptotic pathway. In another aspect, the model system is a model system for the extrinsic apoptotic pathway.

In certain embodiments, the invention provides methods of identifying a compound that binds to a ZPA polypeptide, comprising contacting a ZPA polypeptide with a compound and determining whether the compound binds to the ZPA polypeptide. In certain embodiments, the invention provides methods for identifying a compound which modulates the activity of a ZPA polypeptide, comprising contacting a ZPA polypeptide with a compound and determining whether the compound modulates the activity of the ZPA polypeptide.

In certain embodiments, the invention provides methods for identifying agents that modulate apoptosis. In one embodiment, a method for identifying an agent for reducing or preventing apoptosis is provided, comprising administering at least one agent to a zebrafish and determining whether apoptosis is reduced or prevented. In one aspect, the method further comprises determining the presence or amount of apoptosis in the zebrafish prior to administering the at least one agent. In another aspect, the method further comprises stimulating apoptosis in the zebrafish prior to administering the at least one agent. In another aspect, the agent reduces or prevents apoptosis through the intrinsic apoptotic pathway. In another aspect, the agent reduces or prevents apoptosis through the extrinsic apoptotic pathway. In another aspect, the expression and/or activity of one or more B2R proteins in the zebrafish is increased relative to the expression or activity of the one or more B2R proteins in a wild-type zebrafish. In another aspect, one or more B2R proteins is not expressed in the zebrafish. In another aspect, the expression and/or activity of one or more B2R proteins is reduced in the zebrafish relative to the expression and/or activity of the one or more B2R proteins in a wild-type zebrafish. In another aspect, the agent is selected from an antibody, an antigen-binding antibody fragment, an aptamer, and a small molecule. In another aspect, the zebrafish is a larval zebrafish. In another aspect, the determining step comprises microscopic examination of cell viability. In another aspect, the determining step comprises determining caspase activation.

In another embodiment, a method for identifying an agent for initiating and/or stimulating apoptosis is provided, comprising administering at least one agent to a zebrafish and determining whether apoptosis is initiated or increased. In one aspect, the method further comprises determining the presence or amount of apoptosis in the zebrafish prior to administering the at least one agent. In another aspect, the method further comprises preventing and/or decreasing apoptosis in the zebrafish prior to administering the at least one agent. In another aspect, the agent initiates and/or stimulates apoptosis through the intrinsic apoptotic pathway. In another aspect, the agent initiates and/or stimulates apoptosis through the extrinsic apoptotic pathway. In another aspect, the expression and/or activity of one or more B2R proteins in the zebrafish is increased relative to the expression or activity of the one or more B2R proteins in a wild-type zebrafish. In another aspect, one or more B2R proteins is not expressed in the zebrafish. In another aspect, the expression and/or activity of one or more B2R proteins is reduced in the zebrafish relative to the expression and/or activity of the one or more B2R proteins in a wild-type zebrafish. In another aspect, the agent is selected from an antibody, an antigen-binding antibody fragment, an aptamer, and a small molecule. In another aspect, the zebrafish is a larval zebrafish. In another aspect, the determining step comprises microscopic examination of cell viability. In another aspect, the determining step comprises determining caspase activation.

In certain embodiments, the invention provides further methods for identifying agents for modulating apoptosis. In one embodiment, a method for identifying an agent for preventing or decreasing apoptosis is provided, comprising contacting at least one polypeptide encoded by an amino acid sequence selected from SEQ ID NOs: 1, 5, 7, and 9 with the agent and determining the ability of the agent to block or decrease activity of the at least one polypeptide. In another embodiment, a method for identifying an agent for preventing or decreasing apoptosis is provided, comprising contacting a cell comprising at least one polynucleotide encoded by a nucleotide sequence selected from SEQ ID NOs: 2, 6, 8, and 10 with the agent and determining the ability of the agent to prevent or decrease expression of the at least one polynucleotide.

In another embodiment, a method for identifying an agent for initiating or stimulating apoptosis is provided, comprising contacting at least one polypeptide encoded by an amino acid sequence selected from SEQ ID NOs: 1, 5, 7, and 9 with the agent and determining the ability of the agent to stimulate or increase activity of the at least one polypeptide. In one embodiment, a method for identifying an agent for initiating or stimulating apoptosis, comprising contacting a cell comprising at least one polynucleotide encoded by a nucleotide sequence selected from SEQ ID NOs: 2, 6, 8, and 10 with the agent and determining the ability of the agent to stimulate or increase expression of the at least one polynucleotide.

In certain embodiments, the invention provides methods of treatment. In one embodiment, a method of treating an apoptosis-related disorder is provided, comprising administering to a patient at least one polypeptide encoded by an amino acid sequence selected from SEQ ID NOs: 1, 5, 7, and 9. In another embodiment, a method of treating an apoptosis-related disorder is provided, comprising administering to a patient in need of such treatment an effective amount of at least one polypeptide encoded by an amino acid sequence selected from SEQ ID NOs: 1, 5, 7, and 9, whereby the apoptosis-related disorder is treated in the patient. In another embodiment, a method of treating an apoptosis-related disorder is provided, comprising administering to a patient an agonist of at least one polypeptide encoded by an amino acid sequence selected from SEQ TD NOs: 1, 5, 7, and 9. In another embodiment, a method of treating an apoptosis-related disorder is provided, comprising administering to a patient in need of such treatment an effective amount of an agonist of at least one polypeptide encoded by an amino acid sequence selected from SEQ ID NOs: 1, 5, 7, and 9, whereby the apoptosis-related disorder is treated in the patient. In another embodiment, a method of treating an apoptosis-related disorder is provided, comprising administering to a patient an antagonist of at least one polypeptide encoded by an amino acid sequence selected from SEQ ID NOs: 1, 5, 7, and 9. In another embodiment, a method of treating an apoptosis-related disorder is provided, comprising administering to a patient in need of such treatment an effective amount of an antagonist of at least one polypeptide encoded by an amino acid sequence selected from SEQ ID NOs: 1, 5, 7, and 9, whereby the apoptosis-related disorder is treated in the patient. In one aspect, the antagonist is selected from an aptamer, an antibody, an antigen-binding antibody fragment, and a small molecule.

In another aspect, the apoptosis-related disorder is selected from a cell proliferative disorder, a viral apoptosis disorder, an autoimmune disorder, a hematologic disorder, and a neurological disorder. In one aspect, the apoptosis-related disorder is cancer. In another embodiment, a method of treating an apoptosis-related disorder is provided, comprising administering to a patient at least one polypeptide selected from the group of polypeptides encoded by the polynucleotide sequences of SEQ ID NOs: 2, 6, 8, and 10. In one aspect, the apoptosis-related disorder is selected from a cell proliferative disorder, a viral apoptosis disorder, an autoimmune disorder, a hematologic disorder, and a neurological disorder. In one aspect, the apoptosis-related disorder is cancer.

In certain embodiments, the invention provides compositions for modulating apoptosis. In one embodiment, a composition for increasing apoptosis is provided, comprising a polypeptide encoded by an amino acid sequence selected from SEQ ID NOs: 1, 5, 7, and 9. In one aspect, the composition further comprises a pharmaceutically-acceptable carrier. In another embodiment, a composition for increasing apoptosis is provided, comprising an agonist of a polypeptide encoded by an amino acid sequence selected from SEQ ID NOs: 1, 5, 7, and 9. In another embodiment, a composition for reducing or preventing apoptosis is provided, comprising an antagonist of one or more of SEQ ID NOs: 1, 5, 7, and 9. In one aspect, the antagonist is selected from an antibody, an antigen-binding antibody fragment, an aptamer, and a small molecule. In another aspect, the composition further comprises a pharmaceutically-acceptable carrier. In another embodiment, a composition for reducing or preventing apoptosis is provided, comprising an agent that reduces or inhibits expression of one or more of SEQ ID NOs: 2, 6, 8, and 10. In one aspect, the composition further comprises a pharmaceutically-acceptable carrier.

In certain embodiments, the invention provides methods of treating an apoptosis-related disorder in a subject in need of treatment, comprising administering at least one of the compositions of the invention. In certain embodiments, the invention provides methods of treating an apoptosis-related disorder in a subject in need of treatment, comprising administering an effective amount of at least one of the compositions of the invention, whereby the apoptosis-related disorder is treated in the patient. In certain aspects, the apoptosis-related disorder is selected from a cell proliferative disorder, a viral apoptosis disorder, an autoimmune disorder, a hematologic disorder, and a neurological disorder.

In certain embodiments, the invention provides methods of detecting the presence, severity, and/or predisposition to an apoptosis-related disorder in a subject. In one embodiment, the presence of an apoptosis-related disorder is detected by detecting the presence or amount of a ZPA polypeptide in cells from the subject. In another embodiment, a predisposition to an apoptosis-related disorder is detected by detecting the presence or amount of a ZPA polypeptide in cells from the subject. In another embodiment, the severity of an apoptosis-related disorder is detected by detecting the presence or amount of a ZPA polypeptide in cells from the subject. In another embodiment, the presence of an apoptosis-related disorder is detected by detecting the presence or amount of a ZPA polypeptide homolog in cells from the subject. In another embodiment, a predisposition to an apoptosis-related disorder is detected by detecting the presence or amount of a ZPA polypeptide homolog in cells from the subject. In another embodiment, the severity of an apoptosis-related disorder is detected by detecting the presence or amount of a ZPA polypeptide homolog in cells from the subject. In certain aspects, the apoptosis-related disorder is selected from a cell proliferative disorder, a viral apoptosis disorder, an autoimmune disorder, a hematologic disorder, and a neurological disorder.

In another embodiment, the presence of an apoptosis-related disorder is detected by detecting the presence or amount of expression of a ZPA polynucleotide in cells from the subject. In another embodiment, a predisposition to an apoptosis-related disorder is detected by detecting the presence or amount of expression of a ZPA polynucleotide in cells from the subject. In another embodiment, the severity of an apoptosis-related disorder is detected by detecting the presence or amount of expression of a ZPA polynucleotide in cells from the subject. In another embodiment, the presence of an apoptosis-related disorder is detected by detecting the presence or amount of expression of a ZPA polynucleotide homolog in cells from the subject. In another embodiment, a predisposition to an apoptosis-related disorder is detected by detecting the presence or amount of expression of a ZPA polynucleotide homolog in cells from the subject. In another embodiment, the severity of an apoptosis-related disorder is detected by detecting the presence or amount of expression of a ZPA polynucleotide homolog in cells from the subject. In certain aspects, the apoptosis-related disorder is selected from a cell proliferative disorder, a viral apoptosis disorder, an autoimmune disorder, a hematologic disorder, and a neurological disorder.

In certain embodiments, the invention also provides kits and articles of manufacture for the compounds and compositions described herein, in any useful combination. In one embodiment, a kit is provided comprising one or more of the compositions of the invention and instructions for use. In one aspect the use is a therapeutic use. In another aspect the use is a diagnostic use. In another aspect the use is a research use. In another embodiment, a kit is provided comprising an in vitro intrinsic apoptotic pathway model system and instructions for its use. In one aspect the use is a diagnostic use. In another aspect the use is a research use. In another embodiment, a kit is provided comprising a zebrafish intrinsic apoptotic pathway model system and instructions for its use. In one aspect the use is a diagnostic use. In another aspect the use is a research use. In another embodiment, the invention provides an article of manufacture comprising: (a) a composition comprising one or more ZPA polypeptides, agonists, and antagonists, (b) a container containing said composition; and (c) a label affixed to the container, or a package insert included in the container referring to the use of the composition in the treatment of an apoptosis-related disorder.

### BRIEF DESCRIPTION OF THE DRAWINGS

FIG. 1 shows precision-recall plots for Hidden Markov Models (HMM) constructed from PROSITE patterns and matrices, as described in Example 1(b). The precision and recall of PROSITE patterns are indicated by diamonds; those for pattern-derived HMMs at various scores are plotted as lines; and hourglasses denote the precision and recall at the HMM score thresholds used herein.

FIG. 2 shows an alignment of the BH3 domains of known and candidate Bcl-2-related (“B2R”) proteins, as described in Example 2. Amino acids with similar physicochemical properties are shaded similarly, in accordance with standard ClustalX color patterns.

FIG. 3A depicts an alignment of known and candidate zebrafish B2R proteins with human (h), mouse (m), and chicken (gg) counterparts, as described in Example 2(g). Pro-survival proteins appear as the topmost and middle unshaded sections; BH3-only proteins appear as the bottom-most unshaded section; and the remaining shaded sections are multidomain pro-apoptotic proteins. FIG. 3B shows an alignment of the BH3 domains of human, mouse, and zebrafish BH3-only proteins grouped according to gene, as discussed in Example 2. Amino acids with similar physicochemical properties are shaded similarly. FIGS. 3C and 3D depict the results of experiments described in Example 3(a). FIG. 3C depicts the electrophoretic results of stage-specific RT-PCR, showing that most zebrafish Bcl-2 family members were expressed at consistent levels from the maternal contribution until 72 hours post fertilization (hpf). FIG. 3D depicts the electrophoretic results of tissue-specific RT-PCR, showing expression of many zebrafish Bcl-2 family members in a variety of adult zebrafish tissues.

FIGS. 4A and 4B depict graphs showing the results of ectopic zebrafish B2R protein expression in vivo, as described in Example 3(b). With the exception of zBad, zBok1, and zBok2, ectopic expression of each pro-apoptotic zBcl-2 family member induced death in a dose-dependent manner. FIG. 4C shows brightfield microscopic images (left panels) and immunofluorescent staining for activated caspase-3 (right panels) in zebrafish embryos injected with synthetic zebrafish B2R proteins or a green fluorescent protein (GFP) control, as described in Example 3(b). FIG. 4D shows a graphical depiction of the data obtained from experiments described in Example 3(c). The percent of surviving embryos is plotted for the indicated combinations of ectopically expressed zebrafish B2R proteins. The top of the graph shows the total number of embryos examined for each combination.

FIGS. 5A-5F depict the results of experiments described in Example 3(d). FIG. 5A shows immunostaining for caspase-3 activity in untreated (left panels) or gamma-irradiated (right panels) zebrafish embryos ectopically expressing one of the zebrafish pro-survival B2R proteins (zBlp1, zMcl-1a, zMcl-1b, or zBlp2) or a control (WT (no injection) or GFP). FIG. 5B shows immunostaining for caspase-3 activity in untreated (left panels) or gamma-irradiated (right panels) zebrafish injected with a morpholino to p53 or a control morpholino alone or in combination with morpholinos to zBax or zBak. FIG. 5C shows a graph quantifying the fluorescence from zBax and zBak single and double knockdowns. FIG. 5D shows immunostaining for caspase-3 activity in untreated (left panels) or gamma-irradiated (right panels) zebrafish embryos injected with a control morpholino or with a morpholino to a zebrafish BH3-only B2R protein (zBid, zBad1, zBmf1, zNoxa, zPuma, or zBik). FIG. 5E shows immunostaining for caspasc-3 activity in untreated (left panels) or gamma-irradiated (right panels) zebrafish embryos uninjected or injected with a control morpholino or a morpholino against p53. FIG. 5F graphically depicts the results of quantitative PCR analysis of the increase in zPuma or zNoxa transcription in gamma-irradiated zebrafish embryos untreated or treated with a control or p53 morpholino.

FIG. 6A depicts the results of experiments described in Example 3(e). The figure shows a graph depicting the percent survival of zebrafish embryos subjected to morpholino knockdown of zMcl-1a, zMcl-1b, and/or B1p2. FIGS. 6B and 6C depict the results of experiments described in Example 3(f). The figures show graphs depicting the percent survival of zebrafish embryos subjected to morpholino knockdown of zMcl-1a and/or zMcl-1b and Apo2L-induced apoptosis (either with zebrafish Apo2L ortholog DL1b, or with another Apo2L pathway-related molecule such as zDL1a, zDL2, zDL3, zTNF1, zTNF2, or zFasL).

### DETAILED DESCRIPTION OF THE INVENTION

Applicants, using customized searching techniques, have identified five zebrafish genes previously unknown to be related to the Bcl-2 family of proteins, four of which represent Bcl-2 family members not previously identified in the zebrafish: Bak, Bik, Puma, and Bim. Applicants also herein characterize for the first time the functional activities of certain zebrafish Bcl-2-related (“B2R”) proteins, and demonstrate the existence and function of the intrinsic apoptotic pathway in zebrafish, and the utility of the zebrafish as a model system for the intrinsic apoptotic pathway. Applicants' invention permits the identification of new agents and therapeutics to prevent, decrease, initiate and/or stimulate apoptosis and new methods of studying the role of the Bcl-2 genes and/or the intrinsic apoptotic pathway in apoptosis-related disorders. Applicants' invention also provides new therapeutics for and methods of treating diseases or disorders associated with or caused by aberrant apoptosis.

As described herein, SEQ ID NOs: 1, 3, 5, 7, and 9 (encoded by, respectively, SEQ ID NOs: 2, 4, 6, 8, and 10) are homologous to certain human members of the Bcl-2 family of proteins involved in the intrinsic apoptotic pathway. SEQ ID NO: 1 is a zebrafish protein with sequence identity to human Bak, a multidomain pro-apoptotic protein. SEQ ID NO: 3 is a zebrafish protein with sequence identity to human Bad, a BH3-only pro-apoptotic protein. SEQ ID NO: 5 is a zebrafish protein with sequence identity to human Bik, a BH3-only pro-apoptotic protein. SEQ TD NO: 7 is a zebrafish protein with sequence identity to human Puma, a BH3-only pro-apoptotic protein. SEQ ID NO: 9 is a zebrafish protein with sequence identity to Bmf, a BH3-only pro-apoptotic protein. Applicants have also identified a zebrafish homolog of human Bim, a BH3-only pro-apoptotic protein, but, as described in Example 2(d), the gene could not be cloned due to an apparent error in the current construction of the zebrafish genome.

SEQ ID NOs: 2, 6, 8, and 10 (encoding the proteins of SEQ ID NOs: 1, 5, 7, and 9) were previously identified as part of the zebrafish genome project, but until Applicants' work had not been (1) identified as encoding homologs of human Bcl-2 family members, or (2) implicated as encoding members of one or more apoptosis pathways. Applicants identified SEQ ID NOs: 1, 5, 7, and 9 as zebrafish homologs of human Bak, Bik, Puma, and Bmf, respectively, as described herein, by both sequence identity/similarity and by functional analysis.

The invention therefore provides in one embodiment proteins selected from SEQ ID NOs: 1, 3, 5, 7, and 9 which are zebrafish B2R multidomain or BH3-only pro-apoptotic proteins, compositions containing them, and methods of using the proteins and compositions. The invention also provides in another embodiment polynucleotides selected from SEQ ID NOs: 2, 4, 6, 8, and 10 which encode zebrafish B2R multidomain or BH3-only pro-apoptotic proteins, compositions containing them, and methods of using the polynucleotides and compositions. In another embodiment, variant proteins are provided comprising one or more amino acid additions, deletions, or mutations from a sequence selected from SEQ ID NOs: 1, 3, 5, 7, and 9. In another embodiment, variant polynucleotides are provided comprising one or more nucleotide additions, deletions, or mutations from a sequence selected from SEQ ID NOs: 2, 4, 6, 8, and 10.

The proteins, variant proteins, nucleic acids, and variant nucleic acids of the invention may be used for therapeutic purposes. For example, one or more of the ZPA (“zebrafish pro-apoptosis”) proteins of the invention or variants thereof may be used as a therapeutic to treat an apoptosis-related disorder in which increased apoptosis is desirable (e.g., a cellular proliferation disorder). The invention also provides compositions comprising one or more ZPA proteins of the invention and a pharmaceutically acceptable carrier, optionally including one or more additional therapeutic agents. In another embodiment, one or more of the ZPA nucleic acids of the invention or variants thereof may be used as a therapeutic to treat an apoptosis-related disorder in which increased apoptosis is desirable, e.g., by expressing the nucleic acid in a subject in need of such treatment such that one or more ZPA proteins is expressed in the patient's cells. Zebrafish proteins and nucleic acids may be preferred for use as a therapeutic over any mammalian homologs, e.g., because of a lesser risk of triggering anti-self reactions.

The ZPA proteins of the invention also find utility in methods of identifying agents to initiate, stimulate, inhibit, or block apoptosis. Agonists for one or more ZPA proteins can be identified by their ability to initiate or stimulate the activity of the one or more ZPA proteins in the intrinsic apoptotic pathway. Such stimulation may be, e.g., by activating the ZPA protein or by interfering with one or more molecules that normally inhibit ZPA protein activity, and suitable agonists include, but are not limited to, antibodies and small molecules. Conversely, antagonists for one or more ZPA proteins can be identified by their ability to block or inhibit the activity of the one or more ZPA proteins in the intrinsic apoptotic pathway. Such inhibition may be, e.g., by prevention of the ZPA protein binding to one or more ligands or targets, or by prevention of the activity of the ZPA protein itself, and suitable antagonists include antibodies and antigen-binding fragments thereof, aptamers, and small molecules. Certain appropriate assays to measure ZPA protein activity in the intrinsic apoptosis pathway are described herein. The ZPA protein agonists may be used as therapeutics to treat an apoptosis-related disorder in which increased apoptosis is desirable, and the ZPA protein antagonists may be used as therapeutics to treat an apoptosis-related disorder in which decreased apoptosis is desirable.

The intrinsic apoptotic pathway responds to intracellular signals directing programmed cell death. Dysregulation of this pathway can lead to inappropriate apoptosis or an inappropriate lack of apoptosis, either of which may result in disorders such as cancer. Thus, a greater understanding is needed of the apoptotic pathway and model systems in which the expression and/or activity of one or more pathway components can be perturbed and the repercussions readily examined. In addition to the identification and analysis of the ZPA proteins described herein, Applicants also have demonstrated that an intrinsic apoptotic pathway exists in zebrafish similar to the intrinsic apoptotic pathway previously characterized in mammals.

Thus, the invention also provides methods of using the zebrafish as a model system for studying apoptosis. In some embodiments, transgenic zebrafish are provided, in which the expression and/or activity of one or more ZPA proteins is modulated relative to a wild-type zebrafish. Such transgenic zebrafish may serve to elucidate the normal operation of zebrafish apoptosis pathways, and also provide a tool for use in screening for agents having agonistic or antagonistic apoptotic activity. In other embodiments, the invention provides transgenic zebrafish in which one or more ZPA proteins are replaced with their counterparts from other organisms, thereby creating a model system to assess whether and to what degree cofactors, environmental factors, or modifications in sequence and structure impact the functioning of a particular apoptotic pathway component. In some embodiments, all of the zebrafish intrinsic apoptotic pathway proteins (i.e., all of the B2R proteins) are genetically replaced by intrinsic apoptotic pathway components from another organism (i.e., mammalian or human). Such transgenic zebrafish provide a tool for studying the intrinsic apoptotic pathway that can be examined and manipulated far more readily than it could in the other organism.

In some embodiments, it may be useful to examine the biochemical interactions between intrinsic apoptotic pathway members in the absence of other pathways or stimuli that might interfere with the analysis. Thus, the invention also provides in vitro model systems, whereby the zebrafish intrinsic apoptotic pathway is reconstituted in vitro, optionally with one or more cofactors, reagents, inhibitors, and/or stimulators. In one aspect, the in vitro model system comprises one or more ZPA proteins modified in activity or amount. In another aspect, the in vitro model system comprises one or more B2R proteins modified in activity or amount. In another aspect, the in vitro model system comprises one or more ZPA protein variants. In another aspect, the in vitro model system comprises one or more B2R protein variants. In another aspect, the in vitro model system lacks at least one ZPA protein. In another aspect, the in vitro model system lacks at least one B2R protein. In another aspect, at least one ZPA protein is replaced with a counterpart protein from another organism. In another aspect, at least one B2R protein is replaced with a counterpart protein from another organism.

The ZPA proteins and nucleic acids described herein also find use in detecting an apoptosis-related disorder in a subject. In one embodiment, the presence of an apoptosis-related disorder is detected by detecting the presence or amount of a ZPA polypeptide or a ZPA polypeptide homolog in cells from the subject. In another embodiment, a predisposition to an apoptosis-related disorder is detected by detecting the presence or amount of a ZPA polypeptide or a ZPA polypeptide homolog in cells from the subject. In another embodiment, the severity of an apoptosis-related disorder is detected by detecting the presence or amount of a ZPA polypeptide or a ZPA polypeptide homolog in cells from the subject.

In another embodiment, the presence of an apoptosis-related disorder is detected by detecting the presence or amount of expression of a ZPA polynucleotide or a ZPA polynucleotide homolog in cells from the subject. In another embodiment, a predisposition to an apoptosis-related disorder is detected by detecting the presence or amount of expression of a ZPA polynucleotide or a ZPA polynucleotide homolog in cells from the subject. In another embodiment, the severity of an apoptosis-related disorder is detected by detecting the presence or amount of expression of a ZPA polynucleotide or a ZPA polynucleotide homolog in cells from the subject.

The invention also provides kits and articles of manufacture for the compounds and compositions described herein, in any useful combination. For example, a kit is provided comprising one or more of the compositions of the invention and instructions for use, e.g., therapeutic, diagnostic, and/or research use. In another example, a kit is provided comprising an in vitro or zebrafish intrinsic apoptotic pathway model system and instructions for its use in research or screening for agents to modulate apoptosis.

Details of these methods, compositions, model systems, kits, and articles of manufacture are provided herein.

### DEFINITIONS

The terms “Bcl-2-related protein”, “Bcl-2-related polypeptide” and “B2R protein” as used herein include native sequence polypeptides, polypeptide variants and fragments of native sequence polypeptides and polypeptide variants (which are further defined herein), unless specified otherwise. B2R proteins can be obtained from various species, e.g., humans, by using antibodies according to this invention or by recombinant or synthetic methods, including using deposited nucleic acid molecules. In certain embodiments, B2R proteins are obtained from zebrafish. When obtained from zebrafish, B2R proteins are designated as “zB2R proteins.” B2R proteins include, but are not limited to, Bcl-2-like survival factors (including, but not limited to, Bcl2, Bcl-x_{L}, Bcl-w, Mcl-1, A1/Bfl-1, NR-13, BHRF1, LMW5-HL, ORF16, v-Bcl-2(KSHV), E1B-19K, CED-9, Boo/DIVA/Bcl2-L-10, Bcl-B); to pro-apoptotic multidomain factors (including, but not limited to, Bax, BpR, Bak, Bok/Mtd, Bcl-Rambo, Bcl-x_{S}, and Bcl-G); and to pro-apoptotic BH3-only factors (including, but not limited to, Bik/Nbk, Blk, Hrk/DP5, BNIP3, BimL/Bod, Bad, Bid, EGL-1, Noxa, PUMA/Bbc3, Bmf, Bnip1, Bnip2, and Bnip3). zB2R proteins include, but are not limited to, pro-survival factors (including, but not limited to, zBlp1, zBlp2, zMcl-1a, zMcl-1b, and zNR13); to pro-apoptotic multidomain factors (including, but not limited to, zBak, zBax, zBok1, and zBok2); and to pro-apoptotic BH3-only factors (including, but not limited to, zBad1, zBad2, zBid, zBik, zBmf1, aBmf2, zNoxa, zPuma, and zBim).

The terms “zebrafish pro-apoptosis protein”, “zebrafish pro-apoptosis polypeptide”, “zebrafish pro-apoptotic protein”, “zebrafish pro-apoptotic polypeptide”, “ZPA polypeptide” and “ZPA protein” are used interchangeably herein, and include native sequence polypeptides, polypeptide variants and fragments of native sequence polypeptides and polypeptide variants (which are further defined herein), unless specified otherwise. ZPA proteins can be obtained from zebrafish by using antibodies according to this invention or by recombinant or synthetic methods, including using deposited nucleic acid molecules. ZPA proteins include the zebrafish proteins identified herein, e.g., zBak (SEQ ID NO: 1), zBik (SEQ ID NO: 5), zBim, zPuma (SEQ ID NO: 7), and zBmf2 (SEQ ID NO: 9).

The terms “intrinsic apoptotic pathway”, “intrinsic apoptosis pathway” or “intrinsic pathway” are used interchangeably herein, and refer to a cellular biochemical pathway resulting in apoptosis of the cell which is initiated intracellularly.

The terms “extrinsic apoptotic pathway”, “extrinsic apoptosis pathway” and “extrinsic pathway” are used interchangeably herein, and refer to a cellular biochemical pathway resulting in apoptosis of the cell which is initiated extracellularly.

As used herein, the term “zebrafish” refers to any fish or strain of fish that is considered to be of the genus and species Danio rerio.

A “native sequence” polypeptide or “native” polypeptide is one which has the same amino acid sequence as a polypeptide (e.g., antibody) derived from nature. A “native sequence” polypeptide is one which has the same amino acid sequence as a polypeptide (e.g., antibody) derived from nature. Such native sequence polypeptides can be isolated from nature or can be produced by recombinant or synthetic means. Thus, a native sequence polypeptide can have the amino acid sequence of a naturally occurring human polypeptide, zebrafish polypeptide, or polypeptide from any other species. A “native sequence” ZPA polypeptide or a “native” ZPA polypeptide comprises a polypeptide having the same amino acid sequence as the corresponding ZPA polypeptide derived from nature. For example, in one embodiment, the nucleic acid sequence encoding a native sequence of the zebrafish ZPA protein zPuma can be found in SEQ ID NO: 8 and Example 2(e).

Such ZPA polypeptides can be isolated from nature or can be produced by recombinant or synthetic means. The term “native sequence” or “native” ZPA polypeptide or protein specifically encompasses naturally-occurring truncated or secreted forms of the ZPA protein, naturally-occurring variant forms (e.g., alternatively spliced forms) and naturally-occurring allelic variants of the polypeptide. In certain embodiments of the invention, the native sequence ZPA polypeptides disclosed herein are mature or full-length native sequence polypeptides comprising the full-length amino acid sequences set forth herein.

The approximate location of the “signal peptides” of the various ZPA polypeptides disclosed herein can be seen in the present specification and/or the accompanying figures. It is also recognized that, in some cases, cleavage of a signal sequence from a secreted polypeptide is not entirely uniform, resulting in more than one secreted species. These mature polypeptides, where the signal peptide is cleaved within no more than about 5 amino acids on either side of the C-terminal boundary of the signal peptide as identified herein, and the polynucleotides encoding them, are contemplated by the present invention.

A “ZPA polypeptide variant” or “ZPA protein variant” means a ZPA polypeptide having at least about 80% amino acid sequence identity with a full-length native sequence ZPA polypeptide sequence as disclosed herein, or any fragment of a full-length ZPA polypeptide sequence as disclosed herein (such as those encoded by a nucleic acid that represents only a portion of the complete coding sequence for a full-length ZPA polypeptide). Such ZPA polypeptide variants include, for instance, ZPA polypeptides wherein one or more amino acid residues are added, or deleted, at the N- or C-terminus of the full-length native amino acid sequence. Ordinarily, a ZPA polypeptide variant will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence ZPA polypeptide sequence as disclosed herein, or any specifically defined fragment of a full-length ZPA polypeptide sequence as disclosed herein. Ordinarily, ZPA variant polypeptides are at least about 10 amino acids in length, alternatively at least about 20, 30, 40, 50, 60, 70, 80, 90, 100, 110, 120, 130, 140, 150, 160, 170, 180, 190, 200, 210 amino acids in length, or more. Optionally, ZPA variant polypeptides will have no more than one conservative amino acid substitution as compared to the native ZPA polypeptide sequence, alternatively no more than 2, 3, 4, 5, 6, 7, 8, 9, or 10 conservative amino acid substitution as compared to the native ZPA polypeptide sequence.

“Percent (%) amino acid sequence identity” with respect to the ZPA polypeptide sequences identified herein is defined as the percentage of amino acid residues in a candidate sequence that are identical with the amino acid residues in the specific ZPA polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or can be compiled from the publicly available source code. The ALIGN-2 program should be compiled for use on a UNIX operating system, e.g., digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

As used herein, “conserved synteny” refers to evidence that the human locus evolved from the zebrafish locus, e.g., similar neighboring genes on one or both sides of a ZPA gene and a human gene to which the ZPA gene is believed to be homologous.

“ZPA variant polynucleotide” or “ZPA variant nucleic acid sequence” means a nucleic acid molecule which encodes a ZPA polypeptide, preferably an active ZPA polypeptide, as defined herein and which has at least about 80% nucleic acid sequence identity with a nucleotide acid sequence encoding a full-length native sequence ZPA polypeptide sequence as disclosed herein, or any fragment of a full-length ZPA polypeptide sequence as disclosed herein. Ordinarily, a ZPA variant polynucleotide will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding a full-length native sequence ZPA polypeptide sequence as disclosed herein, or any fragment of a full-length ZPA polypeptide sequence as disclosed herein. Variants do not encompass the native nucleotide sequence.

Ordinarily, ZPA variant polynucleotides are at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, or 625 nucleotides in length, wherein in this context the term “about” means the referenced nucleotide sequence length plus or minus 10% of that referenced length.

“Percent (%) nucleic acid sequence identity” with respect to ZPA-encoding nucleic acid sequences identified herein is defined as the percentage of nucleotides in a candidate sequence that are identical with the nucleotides in the ZPA nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNA STAR) software. For purposes herein, however, % nucleic acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or can be compiled from the publicly available source code. The ALIGN-2 program should be compiled for use on a UNIX operating system, e.g., digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. Unless specifically stated otherwise, all % nucleic acid sequence identity values used herein are obtained as described in the immediately preceding paragraph using the ALIGN-2 computer program.

In other embodiments, ZPA variant polynucleotides are nucleic acid molecules that encode a ZPA polypeptide and which are capable of hybridizing, e.g., under stringent hybridization and wash conditions, to nucleotide sequences encoding a full-length ZPA polypeptide as disclosed herein. ZPA variant polypeptides can be those that are encoded by a ZPA variant polynucleotide.

The term “full-length coding region” when used in reference to a nucleic acid encoding a ZPA polypeptide refers to the sequence of nucleotides which encode the full-length ZPA polypeptide of the invention (which is herein often shown between start and stop codons, inclusive thereof).

“Isolated,” when used to describe the various ZPA polypeptides disclosed herein, means polypeptide that has been identified and separated and/or recovered from a component of its natural environment. Contaminant components of its natural environment are materials that would typically interfere with diagnostic or therapeutic uses for the polypeptide, and can include enzymes, hormones, and other proteinaceous or non-proteinaceous solutes. In certain embodiments, the polypeptide will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue and/or silver stain. Isolated polypeptide includes polypeptide in situ within recombinant cells, since at least one component of the ZPA polypeptide natural environment will not be present. Ordinarily, however, isolated polypeptide will be prepared by at least one purification step.

An “isolated” ZPA polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, can be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” can be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “epitope tagged” when used herein refers to a chimeric polypeptide comprising a ZPA polypeptide or anti-ZPA antibody fused to a “tag polypeptide”. The tag polypeptide has enough residues to provide an epitope against which an antibody can be made, yet is short enough such that it does not interfere with activity of the polypeptide to which it is fused. In certain embodiments, the tag polypeptide also is fairly unique so that the antibody does not substantially cross-react with other epitopes. Suitable tag polypeptides generally have at least six amino acid residues and usually between about 8 and 50 amino acid residues (in certain embodiments, between about 10 and 20 amino acid residues). Polypeptides and antibodies of this invention that are epitope-tagged are contemplated.

“Biologically active” and “biological activity” and “biological characteristics” with respect to a ZPA polypeptide means (1) having the ability to initiate or stimulate apoptosis in vivo or ex vivo; (2) having the ability to specifically bind to an upstream and/or downstream member of the intrinsic apoptotic pathway; and/or (3) having the ability to otherwise modulate ZPA signaling or ZPA activity, except where specified otherwise.

“Biologically active” and “biological activity” and “biological characteristics” with respect to a modified ZPA polypeptide means (1) having the ability to initiate or stimulate apoptosis in vivo or ex vivo; (2) having the ability to specifically bind to an upstream and/or downstream member of the intrinsic apoptotic pathway; and/or (3) having the ability to otherwise modulate ZPA signaling or ZPA activity, except where specified otherwise.

“Biologically active” and “biological activity” and “biological characteristics” with respect to an anti-ZPA antibody of this invention means (1) having the ability to partially or fully block, inhibit or neutralize a biological activity of a native ZPA polypeptide (either in an antagonistic or blocking manner); (2) having the ability to specifically bind a ZPA polypeptide; and/or (3) having the ability to modulate ZPA signaling or ZPA activity, except where specified otherwise. In one embodiment, an antibody of this invention binds to a ZPA protein with an affinity of at least 1 uM or less, 100 nm or less, 50 nm or less, 10 nm or less, 5 nM or less, 1 nm or less. As used herein, “antibody variable domain” refers to the portions of the light and heavy chains of antibody molecules that include amino acid sequences of Complementary Determining Regions (CDRs; ie., CDR1, CDR2, and CDR3), and Framework Regions (FRs). V_{H} refers to the variable domain of the heavy chain. V_{L} refers to the variable domain of the light chain. According to the methods used in this invention, the amino acid positions assigned to CDRs and FRs are defined according to Kabat (Sequences of Proteins of Immunological Interest (National Institutes of Health, Bethesda, Md., 1987 and 1991)). Amino acid numbering of antibodies or antigen binding fragments is also according to that of Kabat.

As used herein, “codon set” refers to a set of different nucleotide triplet sequences used to encode desired variant amino acids. A set of oligonucleotides can be synthesized, for example, by solid phase synthesis, containing sequences that represent all possible combinations of nucleotide triplets provided by the codon set and that will encode the desired group of amino acids. A standard form of cod on designation is that of the IUB code, which is known in the art and described herein.

“Heterologous DNA” is any DNA that is introduced into a host cell. The DNA can be derived from a variety of sources including genomic DNA, cDNA, synthetic DNA and fusions or combinations of these. The DNA can include DNA from the same cell or cell type as the host or recipient cell or DNA from a different cell type, for example, from a mammal or plant. The DNA can, optionally, include marker or selection genes, for example, antibiotic resistance genes, temperature resistance genes, etc. Host cells encoding heterologous DNAs comprising the polypeptides and antibodies of this invention are contemplated as well as their use.

As used herein, “library” refers to a plurality of polypeptides (for example, antibody or antibody fragment sequences), or the nucleic acids that encode these sequences, the sequences being different in the combination of variant amino acids that are introduced into these sequences according to the methods of the invention.

“Phage display” is a technique by which variant polypeptides are displayed as fusion proteins to a coat protein on the surface of phage, e.g., filamentous phage, particles. A utility of phage display lies in the fact that large libraries of randomized protein variants can be rapidly and efficiently sorted for those sequences that bind to a target molecule with high affinity. Display of peptide and protein libraries on phage has been used for screening millions of polypeptides for ones with specific binding properties. Polyvalent phage display methods have been used for displaying small random peptides and small proteins through fusions to either gene III or gene VIII of filamentous phage. Wells and Lowman, Curr. Opin. Struct. Biol., 3:355-362 (1992), and references cited therein. In monovalent phage display, a protein or peptide library is fused to a gene III or a portion thereof, and expressed at low levels in the presence of wild type gene III protein so that phage particles display one copy or none of the fusion proteins. Avidity effects are reduced relative to polyvalent phage so that sorting is on the basis of intrinsic ligand affinity, and phagemid vectors are used, which simplify DNA manipulations. Lowman and Wells, Methods: A companion to Methods in Enzymology, 3:205-0216 (1991).

A “phagemid” is a plasmid vector having a bacterial origin of replication, e.g., Co1E1, and a copy of an intergenic region of a bacteriophage. The phagemid can be used on any known bacteriophage, including filamentous bacteriophage and lambdoid bacteriophage. The plasmid will also generally contain a selectable marker for antibiotic resistance. Segments of DNA cloned into these vectors can be propagated as plasmids. When cells harboring these vectors are provided with all genes necessary for the production of phage particles, the mode of replication of the plasmid changes to rolling circle replication to generate copies of one strand of the plasmid DNA and package phage particles. The phagemid can form infectious or non-infectious phage particles. This term includes phagemids which contain a phage coat protein gene or fragment thereof linked to a heterologous polypeptide gene as a gene fusion such that the heterologous polypeptide is displayed on the surface of the phage particle.

The term “phage vector” means a double stranded replicative form of a bacteriophage containing a heterologous gene and capable of replication. The phage vector has a phage origin of replication allowing phage replication and phage particle formation. The phage can be a filamentous bacteriophage, such as an M13, fl, fd, Pf3 phage or a derivative thereof, or a lambdoid phage, such as lambda, 21, phi80, phi81, 82, 424, 434, etc., or a derivative thereof.

The term “proteoglycan” refers to a molecule where at least one glycosaminoglycan side chain is covalently attached to the protein core of the molecule. A proteoglycan synthesis deficient cell line according to this invention includes a cell line that is deficient in galactosyltransferase I. According to one embodiment, the cell line is a CHO-psbg cell line.

The term “antagonist” is any molecule that partially or fully blocks, inhibits, or neutralizes a biological activity of a native ZPA polypeptide and that specifically binds to a native ZPA polypeptide. According to one embodiment, the antagonist is a polypeptide. According to another embodiment, an antibody of the invention can inhibit the binding of the antagonist to the native ZPA polypeptide.

The term “small molecule antagonist” refers to any molecule wherein the molecular weight is 1500 daltons or less and is an antagonist according to this invention. According to one embodiment the small molecule antagonist is below about 500 Daltons.

According to one embodiment, the antagonist blocks, inhibits, decreases, or neutralizes apoptosis in cells expressing at least one native ZPA polypeptide. Suitable antagonists include antibodies, antigen-binding antibody fragments, amino acid sequence variants of native ZPA polypeptides, peptides of this invention, aptamers, etc. Methods for identifying antagonists of a ZPA polypeptide can comprise contacting a ZPA polypeptide with a candidate antagonist molecule and measuring a detectable change in one or more biological activities associated with the ZPA polypeptide.

The term “aptamer” refers to a nucleic acid molecule that is capable of binding to a target molecule, such as a ZPA polypeptide. The generation and therapeutic use of aptamers are well established in the art. See, e.g., U.S. Pat. No. 5,475,096, and the therapeutic efficacy of Macugen® (Eyetech, New York) for treating age-related macular degeneration.

The terms “potentiator” and “agonist” refer to any molecule that enhances a biological activity of a native ZPA polypeptide, wherein the potentiator initiates and/or stimulates apoptosis. In one embodiment, an agonist specifically binds to a native ZPA polypeptide and enhances a biological activity of that native ZPA polypeptide. In another embodiment, an agonist stimulates the transcription and/or translation of a polynucleotide encoding a native ZPA polypeptide such that the expression of the native ZPA polypeptide is increased. In another embodiment, an agonist inhibits the normal functioning of an inhibitor of a native ZPA polypeptide. It is understood that the foregoing embodiments are not mutually exclusive, such that an agonist may, e.g., specifically bind to a native ZPA polypeptide and enhance a biological activity of that native ZPA polypeptide while also inhibiting the normal functioning of an inhibitor of a native ZPA polypeptide. Methods for identifying agonists of a ZPA polypeptide can comprise contacting a molecule that binds ZPA with a ZPA polypeptide and the candidate agonist and measuring a detectable change in one or more biological activities associated with the ZPA polypeptide (e.g., increased caspase activation or increased rate or amount of apoptosis).

“Treating” or “treatment” or “alleviation” refers to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented. These terms indicate the therapeutic and prophylactic uses herein are successful if they ameliorate, lessen or decrease the symptoms, complications or other problems associated with a disease or ameliorate, lessen or decrease the chance of onset or frequency of the symptoms, complications or other problems associated with a disease.

A subject or mammal is successfully “treated” for a cancer if, after receiving a therapeutic amount of an antagonist according to the methods of the present invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent an anti-ZPA antibody or ZPA-binding oligopeptide can prevent growth and/or kill existing cancer cells, it can be cytostatic and/or cytotoxic. Reduction of these signs or symptoms can also be felt by the patient.

A subject or mammal is successfully “treated” for an apoptosis-related disorder if, after receiving a therapeutic amount of an antagonist or agonist according to the methods of the present invention, the patient shows observable and/or measurable modulation of apoptosis, and/or relief to some extent, of one or more of the symptoms associated with the aberrant apoptosis; and improvement in quality of life issues.

The above parameters for assessing successful treatment and improvement in the disease are readily measurable by procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other known methods for monitoring the disease include transrectal ultrasonography (TRUS) and transrectal needle biopsy (TRNB), among other methods well known in the art.

“Chronic” administration refers to administration of the agent(s) in a continuous mode as opposed to an acute mode, so as to maintain the initial therapeutic effect (activity) for an extended period of time. “Intermittent” administration is treatment that is not consecutively done without interruption, but rather is cyclic in nature.

“Mammal” for purposes of the treatment of, alleviating the symptoms of or diagnosis of a cancer refers to any animal classified as a mammal (aka “patient”), including humans, domestic and farm animals, and zoo, sports, or pet animals, such as dogs, cats, cattle, horses, sheep, pigs, goats, rabbits, etc. In certain embodiments, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

By “solid phase” or “solid support” is meant a non-aqueous matrix to which an antibody, an antagonist or a polypeptide of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column) This term also includes a discontinuous solid phase of discrete particles, such as those described in U.S. Pat. No. 4,275,149.

As used herein, the term “immunoadhesin” designates antibody-like molecules that combine the binding specificity of a heterologous protein (an “adhesin”) with the effector functions of immunoglobulin constant domains. Structurally, the immunoadhesins comprise a fusion of an amino acid sequence with the desired binding specificity that is other than the antigen recognition and binding site of an antibody (i.e., is “heterologous”), and an immunoglobulin constant domain sequence. The adhesin part of an immunoadhesin molecule typically is a contiguous amino acid sequence comprising at least the binding site of a receptor or a ligand—such as a portion of a native ZPA protein. The immunoglobulin constant domain sequence in the immunoadhesin can be obtained from any immunoglobulin, such as IgG-1, IgG-2, IgG-3, or IgG-4 subtypes, IgA (including IgA-1 and IgA-2), IgE, IgD, or IgM.

A “liposome” is a small vesicle composed of various types of lipids, phospholipids and/or surfactant which is useful for delivery of a drug (such as a ZPA polypeptide, an antibody thereto or a ZPA-binding oligopeptide) to a mammal. The components of the liposome are commonly arranged in a bilayer formation, similar to the lipid arrangement of biological membranes.

An “effective amount” of a polypeptide, antibody, antagonist or composition as disclosed herein is an amount sufficient to carry out a specifically stated purpose. An “effective amount” can be determined empirically and by known methods relating to the stated purpose.

The term “therapeutically effective amount” refers to an amount of an antibody, polypeptide or antagonist of this invention effective to “treat” a disease or disorder in a mammal (aka patient). In the case of cancer, the therapeutically effective amount of the drug can reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the drug can prevent growth and/or kill existing cancer cells, it can be cytostatic and/or cytotoxic.

A “cytotoxic amount” of a polypeptide, antibody, antagonist or composition of this invention is an amount capable of causing the destruction of a cell, especially tumor, e.g., cancer cell, either in vitro or in vivo. A “cytotoxic amount” of a polypeptide, antibody, antagonist or composition of this invention for purposes of inhibiting, e.g., neoplastic cell growth, can be determined empirically and by methods known in the art.

The term “antibody” is used in the broadest sense and specifically covers, for example, single anti-ZPA polypeptide monoclonal antibodies (including agonist, antagonist, and neutralizing antibodies), anti-ZPA polypeptide antibody compositions with polyepitopic specificity, polyclonal antibodies, single chain anti-ZPA polypeptide antibodies, and fragments of anti-ZPA polypeptide antibodies (see below) as long as they specifically bind a native ZPA polypeptide and/or exhibit a biological activity or immunological activity of this invention. The phrase “functional fragment or analog” of an antibody is a compound having a qualitative biological activity in common with an antibody to which it is being referred. For example, a functional fragment or analog of an anti-ZPA polypeptide antibody can be one which can specifically bind to a ZPA molecule. In one embodiment, the antibody can prevent or substantially reduce the ability of a ZPA molecule to induce apoptosis. The term “immunoglobulin” (Ig) is used interchangeably with “antibody” herein.

An “isolated antibody” is one which has been identified and separated and/or recovered from a component of its natural environment. Contaminant components of its natural environment are materials which would interfere with diagnostic or therapeutic uses for the antibody, and can include enzymes, hormones, and other proteinaceous or nonproteinaceous solutes. In certain embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{II}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the V_{L}, and around about 1-35 (H1), 50-65 (H2) and 95-102 (H3) in the V_{II} (in one embodiment, H1 is around about 31-35); Kabat et al., Sequences of Proteins of Immunological Interest, 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the V_{L}, and 26-32 (H1), 53-55 (H2) and 96-101 (H3) in the V_{H}; Chothia and Lesk J. Mol. Biol. 196:901-917 (1987)).

The term “monoclonal antibody” as used herein refers to an antibody obtained from a population of substantially homogeneous antibodies, i.e., the individual antibodies comprising the population are identical except for possible naturally occurring mutations that can be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they can be synthesized uncontaminated by other antibodies. The modifier “monoclonal” is not to be construed as requiring production of the antibody by any particular method. For example, the monoclonal antibodies useful in the present invention can be prepared by the hybridoma methodology first described by Kohler et al., Nature, 256:495 (1975), or can be made using recombinant DNA methods in bacterial, eukaryotic animal or plant cells (see, e.g., U.S. Pat. No. 4,816,567). The “monoclonal antibodies” can also be isolated from phage antibody libraries using the techniques described in Clackson et al., Nature, 352:624-628 (1991), Marks et al., J. Mol. Biol., 222:581-597 (1991), and the Examples below, for example.

The monoclonal antibodies herein include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit a biological activity of this invention (see U.S. Pat. No. 4,816,567; and Morrison et al., Proc. Natl. Acad. Sci. USA, 81:6851-6855 (1984)). Chimeric antibodies of interest herein include “primatized” antibodies comprising variable domain antigen-binding sequences derived from a non-human primate (e.g. Old World Monkey, Ape etc), and human constant region sequences.

An “intact” antibody is one which comprises an antigen-binding site as well as a C_{L} and at least heavy chain constant domains, C_{H}1, C_{H}2 and C_{H}3. The constant domains can be native sequence constant domains (e.g. human native sequence constant domains) or amino acid sequence variant thereof. In certain embodiments, the intact antibody has one or more effector functions.

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062 [1995]); single-chain antibody molecules; and multispecific antibodies formed from antibody fragments. The expression “linear antibodies” generally refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. In certain embodiments, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

The term “diabodies” refers to small antibody fragments prepared by constructing sFv fragments (see preceding paragraph) with short linkers (about 5-10 residues) between the V_{H} and V_{L} domains such that inter-chain but not intra-chain pairing of the V domains is achieved, resulting in a bivalent fragment, i.e., fragment having two antigen-binding sites. Bispecific diabodics are heterodimers of two “crossover” sFv fragments in which the V_{H} and V_{L} domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

A “species-dependent antibody,” e.g., a mammalian anti-human IgE antibody, is an antibody which has a stronger binding affinity for an antigen from a first mammalian species than it has for a homologue of that antigen from a second mammalian species. Normally, the species-dependent antibody “bind specifically” to a human antigen (i.e., has a binding affinity (Kd) value of no more than about 1×10⁻⁷ M, no more than about 1×10⁻⁸, and no more than about 1×10⁻⁹ M) but has a binding affinity for a homologue of the antigen from a second non-human mammalian species which is at least about 50 fold, or at least about 500 fold, or at least about 1000 fold, weaker than its binding affinity for the human antigen. The species-dependent antibody can be of any of the various types of antibodies as defined above, an in certain embodiments is a humanized or human antibody.

A “ZPA-binding oligopeptide” is an oligopeptide that binds, preferably specifically, to a ZPA polypeptide as described herein. ZPA-binding oligopeptides can be chemically synthesized using known oligopeptide synthesis methodology or can be prepared and purified using recombinant technology. ZPA-binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a ZPA polypeptide as described herein. ZPA-binding oligopeptides can be identified without undue experimentation using known techniques. In this regard, it is noted that techniques for screening oligopeptide libraries for oligopeptides that are capable of specifically binding to a polypeptide target are known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

A polypeptide, antibody, antagonist or composition of this invention “which binds” an antigen of interest, e.g. a ZPA polypeptide, is one that binds the antigen with sufficient affinity such that a polypeptide, antibody, antagonist or composition is useful as a diagnostic and/or therapeutic agent in targeting a cell or tissue expressing the antigen, and does not significantly cross-react with other proteins. In such embodiments, the extent of binding of the polypeptide, antibody, antagonist or composition to a “non-target” protein will be less than about 10% of the binding of the polypeptide, antibody, antagonist or composition to its particular target protein as determined by fluorescence activated cell sorting (FACS) analysis or radioimmunoprecipitation (RIA). With regard to the binding of a polypeptide, antibody, antagonist or composition to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding” or “specifically binds to” or is “specific for” a particular polypeptide or an epitope on a particular polypeptide target as used herein can be exhibited, for example, by a molecule having a Kd for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In one embodiment, the term “specific binding” refers to binding where a molecule binds to a particular polypeptide or epitope on a particular polypeptide without substantially binding to any other polypeptide or polypeptide epitope (e.g., a non-ZPA protein). It is understood that an antibody that specifically binds to a zebrafish native ZPA polypeptide may also bind a non-zebrafish polypeptide homologous to the ZPA polypeptide.

A polypeptide, antibody, antagonist or composition that “inhibits the growth” of tumor cells or a “growth inhibitory” polypeptide, antibody, antagonist or composition is one which results in measurable growth inhibition of cancer cells. In certain embodiments, growth inhibitory polypeptides, antibodies, antagonists or compositions inhibit growth of tumor cells by greater than 20%, from about 20% to about 50%, and by greater than 50% (e.g., from about 50% to about 100%) as compared to the appropriate control, the control typically being tumor cells not treated with the polypeptide, antibody, antagonist or composition being tested. In one embodiment, growth inhibition can be measured at an antibody concentration of about 0.1 to 30 μg/ml or about 0.5 nM to 200 nM in cell culture, where the growth inhibition is determined 1-10 days after exposure of the tumor cells to the antibody. Growth inhibition of tumor cells in vivo can be determined in various ways such as is described in the Experimental Examples section below. The antibody is growth inhibitory in vivo if administration of the anti-ZPA antibody at about 1 μg/kg to about 100 mg/kg body weight results in reduction in tumor size or tumor cell proliferation within about 5 days to 3 months from the first administration of the antibody, for example within about 5 to 30 days.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or U.S. Pat. No. 5,821,337 can be performed. Useful effector cells for such assays include peripheral blood mononuclear cells (PBMC) and Natural Killer (NK) cells. Alternatively, or additionally, ADCC activity of the molecule of interest can be assessed in vivo, e.g., in a animal model such as that disclosed in Clynes et al. (USA) 95:652-656 (1998).

“Fc receptor” or “FcR” describes a receptor that binds to the Fc region of an antibody. In certain embodiments, the FcR is a native sequence human FcR. Moreover, the FcR can be an FcR which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. In certain embodiments, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells can be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, (e.g., as described in Gazzano-Santoro et al., J. Immunol. Methods 202:163 (1996)), can be performed.

An “apoptosis-related disorder” refers to a physiological condition or disease state caused by, prolonged by, or which is characterized by aberrant or misregulated apoptosis. Apoptosis-related disorders include, but are not limited to, cell proliferative disorders, viral apoptosis disorders, autoimmune disorders, hematologic disorders, neurological disorders, and other disorders characterized by an undesirably high or low rate of apoptosis.

The terms “cell proliferative disorder” and “proliferative disorder” refer to disorders that are associated with some degree of abnormal cell proliferation. In one embodiment, the cell proliferative disorder is cancer. Aberrant apoptosis is one cause of abnormal cell proliferation. A number of cancers have been linked to inactivation of one or more pro-apoptotic proteins (e.g., p53 and fas) or overproduction or dysregulation of pro-survival proteins (e.g., Bcl-2).

The terms “cancer” and “cancerous” refer to or describe the physiological condition that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

“Tumor”, as used herein, refers to all neoplastic cell growth and proliferation, whether malignant or benign, and all pre-cancerous and cancerous cells and tissues.

The term “viral apoptosis disorder” refers to or describes aberrant apoptosis in a patient caused by or as a result of viral infection. The term includes both aberrant apoptosis (e.g., decreased or blocked apoptosis) directly caused by an infecting virus, as well as aberrant apoptosis (e.g., increased cell death) caused by excessive, uncontrolled, or mistargeted immune system function in response to a viral infection or by the virus itself. Examples of aberrant (decreased) apoptosis directly caused by an infecting virus include, but are not limited to, production of Bcl-2-like proteins (pro-survival B2R polypeptides) and stimulators of Bcl-2 production by the Epstein-Barr virus such that infected cells do not undergo apoptosis; inactivation or degradation of p53 (a pro-apoptotic polypeptide) by papillomavirus such that infected cells do not undergo apoptosis; and production of an inhibitor of the pro-apoptotic ICE-like proteases by cowpox virus, such that infected cells do not undergo apoptosis. An example of aberrant (increased) apoptosis caused by viral infection is inappropriate expression of fas at the surface of infected helper T cells, which causes those cells to undergo premature apoptosis, thereby eliminating an important component of the immune system. Examples of aberrant apoptosis caused by excessive, uncontrolled, or mistargeted immune system function in response to a viral infection includes the inadvertent killing of uninfected cells neighboring infected cells because the neighboring cells may also have been induced to express fas at the cell surface, and are thus targeted for destruction by apoptosis pathway activation by circulating cytotoxic T lymphocytes.

The term “autoimmune disorder”, refers to a non-malignant disease or disorder arising from and directed against an individual's own tissues. Autoimmune disorders are typically characterized by the failure of autoreactive immune cells to be destroyed by the immune system; autoreactive lymphocytes have been identified that overexpress or otherwise have increased activity of pro-survival apoptotic factors or have reduced expression or activity of pro-apoptotic factors. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia, etc.

The term “hematologic disorder” refers to or describes a disease or disorder characterized by aberrant production of blood cells, or by inappropriate blood flow. Hematologic disorders include, but are not limited to, anemia associated with chronic disease, aplastic anemia, chronic neutropenia, and the myelodysplastic syndromes, myocardial infarction, and stroke.

The terms “neurological disorder” or “neurological disease” refer to or describe a disease or disorder of the central and/or peripheral nervous system and/or specific neurons that is typically characterized by deterioration of nervous tissue or deterioration of communication between cells in nervous tissue. Examples of neurological disorders include, but are not limited to, neurodegenerative diseases (including, but not limited to, Lewy body disease, postpoliomyelitis syndrome, Shy-Draeger syndrome, retinitis pigmentosum olivopontocerebellar atrophy, Parkinson's disease, spinal muscular atrophy, multiple system atrophy, amyotrophic lateral sclerosis, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease), spinocerebellar ataxia, ischemic and hypoxic brain injury, and traumatic and excitotoxic brain damage.

A polypeptide, antibody, antagonist or composition of this invention which “induces cell death” or “induces apoptosis” is one which causes a viable cell to become nonviable. In certain embodiments, the cell is a cancer cell, e.g., a breast, ovarian, stomach, endometrial, salivary gland, lung, kidney, colon, thyroid, pancreatic or bladder cell. Cell death in vitro can be determined in the absence of complement and immune effector cells to distinguish cell death induced by antibody-dependent cell-mediated cytotoxicity (ADCC) or complement dependent cytotoxicity (CDC). Thus, the assay for cell death can be performed using heat inactivated serum (i.e., in the absence of complement) and in the absence of immune effector cells. To determine whether a polypeptide, antibody, antagonist or composition of this invention is able to induce cell death, loss of membrane integrity as evaluated by uptake of propidium iodide (PI), trypan blue (see Moore et al. Cytotechnology 17:1-11 (1995)) or 7AAD can be assessed relative to untreated cells.

A “ZPA-expressing cell” is a cell which expresses an endogenous or transfected ZPA polypeptide. A “ZPA-expressing cancer” is a cancer comprising cells that produce a ZPA polypeptide. A cancer which “overexpresses” a ZPA polypeptide or a homolog thereof is one which has significantly higher levels of ZPA polypeptide or a homolog thereof compared to a noncancerous cell of the same tissue type. Such overexpression can be caused by gene amplification or by increased transcription or translation. ZPA polypeptide or ZPA polypeptide homolog overexpression can be determined in a diagnostic or prognostic assay by evaluating increased levels of the ZPA protein or ZPA protein homolog present in the cell (e.g., via an immunohistochemistry assay, etc.). Alternatively, or additionally, one can measure levels of ZPA polypeptide-encoding or ZPA polypeptide homolog-encoding nucleic acid or mRNA in the cell, e.g., via fluorescent in situ hybridization using a nucleic acid based probe corresponding to a ZPA-encoding nucleic acid or the complement thereof; (FISH; see WO98/45479 published October, 1998), Southern blotting, Northern blotting, or polymerase chain reaction (PCR) techniques, such as real time quantitative PCR(RT-PCR). Aside from the above assays, various in vivo assays are available to the skilled practitioner. For example, one can expose cells within the body of the mammal to an internalizing antibody which is optionally labeled with a detectable label, e.g., a radioactive isotope, and binding of the antibody to a ZPA polypeptide or a ZPA polypeptide homolog in the mammal can be evaluated, e.g., by external scanning for radioactivity or by analyzing a biopsy taken from a mammal previously exposed to the antibody.

The word “label” when used herein refers to a detectable compound or composition which is conjugated directly or indirectly to the polypeptide, antibody, antagonist or composition so as to generate a “labeled” a polypeptide, antibody, antagonist or composition. The label can be detectable by itself (e.g. radioisotope labels or fluorescent labels) or, in the case of an enzymatic label, can catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², P³² and radioactive isotopes of Lu), chemotherapeutic agents e.g. methotrexate, adriamicin, vinca alkaloids (vincristine, vinblastine, etoposide), doxorubicin, melphalan, mitomycin C, chlorambucil, daunorubicin or other intercalating agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON•toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in aberrant cell proliferation, such as, for example, PKC-alpha, Ralf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

A “growth inhibitory agent” when used herein refers to a compound or composition which inhibits growth of a cell, especially a cancer cell, either in vitro or in vivo. Thus, the growth inhibitory agent can be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6,8,11-trihydroxy-8-(hydroxyacetyl)-1-methoxy-5,12-naphthacenedione.

The term “package insert” is used to refer to instructions customarily included in commercial packages of therapeutic products, that contain information about the indications, usage, dosage, administration, contraindications and/or warnings concerning the use of such therapeutic products.

### Compositions and Methods of the Invention

ZPA Polypeptide Variants

In addition to the full-length native sequence ZPA polypeptides described herein, it is contemplated that ZPA polypeptide variants can be prepared. ZPA polypeptide variants can be prepared by introducing appropriate nucleotide changes into the ZPA nucleic acid, and/or by synthesis of the desired ZPA polypeptide. Those skilled in the art will appreciate that amino acid changes can alter post-translational processes of the ZPA polypeptide such as changing the number or position of glycosylation sites or altering membrane anchoring characteristics.

Variations in a native full-length sequence ZPA polypeptide or in various domains of a ZPA polypeptide described herein can be made, for example, using any of the techniques and guidelines for conservative and non-conservative mutations set forth, for instance, in U.S. Pat. No. 5,364,934. Variations can be a substitution, deletion or insertion of one or more codons encoding a ZPA polypeptide that results in a change in the amino acid sequence of the ZPA polypeptide as compared with the native sequence ZPA polypeptide. Optionally the variation is by substitution of at least one amino acid with any other amino acid in one or more of the domains of the ZPA polypeptide. Guidance in determining which amino acid residue can be inserted, substituted or deleted without adversely affecting the desired activity can be found by comparing the sequence of a ZPA polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions can optionally be in the range of about 1 to 5 amino acids. The variation allowed can be determined by systematically making insertions, deletions or substitutions of amino acids in the sequence and testing the resulting variants for activity exhibited by the full-length or mature native sequence.

In particular embodiments, conservative substitutions of interest are shown in Table 1 under the heading of preferred substitutions. If such substitutions result in a change in biological activity, then more substantial changes, denominated exemplary substitutions in Table 1, or as further described below in reference to amino acid classes, are introduced and the products screened.

TABLE 1

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

Substantial modifications in function or immunological identity of a ZPA polypeptide are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:

(1) hydrophobic: norleucine, met, ala, val, leu, ile;
(2) neutral hydrophilic: cys, ser, thr;
(3) acidic: asp, glu;
(4) basic: asn, gln, his, lys, arg;
(5) residues that influence chain orientation: gly, pro; and
(6) aromatic: trp, tyr, phe.

Non-conservative substitutions will entail exchanging a member of one of these classes for another class. Such substituted residues also can be introduced into the conservative substitution sites or into the remaining (non-conserved) sites.

Variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce a ZPA variant DNA.

Scanning amino acid analysis can also be employed to identify one or more amino acids along a contiguous sequence. In certain embodiments, scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244: 1081-1085 (1989)]. Alanine is also typically preferred because it is the most common amino acid. Further, it is frequently found in both buried and exposed positions [Creighton, The Proteins, (W.H. Freeman & Co., N.Y.); Chothia, J. Mol. Biol., 150:1 (1976)]. If alanine substitution does not yield adequate amounts of variant, an isoteric amino acid can be used.

### Modifications of ZPA Polypeptides

Covalent modifications of ZPA polypeptides are included within the scope of this invention. One type of covalent modification includes reacting targeted amino acid residues of a ZPA polypeptide with an organic derivatizing agent that is capable of reacting with selected side chains or the N- or C-terminal residues of the ZPA polypeptide. Derivatization with bifunctional agents is useful, for instance, for crosslinking the ZPA polypeptide to a water-insoluble support matrix or surface for use in the method for purifying anti-ZPA antibodies, and vice-versa. Commonly used crosslinking agents include, e.g., 1,1-bis(diazo-acetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl)dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W.H. Freeman & Co., San Francisco, pp. 79-86 (1983)], acetylation of the N-terminal amine, and amidation of any C-terminal carboxyl group.

Another type of covalent modification of a ZPA polypeptide included within the scope of this invention comprises altering the native glycosylation pattern of the polypeptide. “Altering the native glycosylation pattern” is intended for purposes herein to mean deleting one or more carbohydrate moieties found in the native sequence ZPA polypeptide (either by removing the underlying glycosylation site or by deleting the glycosylation by chemical and/or enzymatic means), and/or adding one or more glycosylation sites that are not present in the native sequence ZPA polypeptide. In addition, the phrase includes qualitative changes in the glycosylation of the native proteins, involving a change in the nature and proportions of the various carbohydrate moieties present.

Addition of glycosylation sites to a ZPA polypeptide can be accomplished by altering the amino acid sequence. The alteration can be made, for example, by the addition of, or substitution by, one or more serine or threonine residues to the native sequence ZPA polypeptide (for O-linked glycosylation sites). The ZPA amino acid sequence can optionally be altered through changes at the DNA level, particularly by mutating the DNA encoding the ZPA polypeptide at preselected bases such that codons are generated that will translate into the desired amino acids.

Another means of increasing the number of carbohydrate moieties on a ZPA polypeptide is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties present on a ZPA polypeptide can be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification of a ZPA polypeptide comprises linking the ZPA polypeptide to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

A ZPA polypeptide of the present invention can also be modified in a way to form a chimeric molecule comprising a ZPA polypeptide fused to another, heterologous polypeptide or amino acid sequence.

In one embodiment, such a chimeric molecule comprises a fusion of a ZPA polypeptide with a protein transduction domain which targets the ZPA polypeptide for delivery to various tissues. In one aspect, the ZPA polypeptide is targeted to the brain across the brain blood barrier, using, for example, the protein transduction domain of human immunodeficiency virus TAT protein (Schwarze et al., 1999, Science 285: 1569-72).

In another embodiment, such a chimeric molecule comprises a fusion of a ZPA polypeptide with a tag polypeptide which provides an epitope to which an anti-tag antibody can selectively bind. The epitope tag is generally placed at the amino- or carboxyl-terminus of the ZPA polypeptide. The presence of such epitope-tagged forms of the ZPA polypeptide can be detected using an antibody against the tag polypeptide. Also, provision of the epitope tag enables the ZPA polypeptide to be readily purified by affinity purification using an anti-tag antibody or another type of affinity matrix that binds to the epitope tag. Various tag polypeptides and their respective antibodies are known in the art. Examples include poly-histidine (poly-His) or poly-histidine-glycine (poly-His-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule can comprise a fusion of a ZPA polypeptide with an immunoglobulin or a particular region of an immunoglobulin. For a bivalent form of the chimeric molecule (also referred to as an “immunoadhesin”), such a fusion could be to the Fc region of an IgG molecule. In one embodiment, the immunoglobulin fusion includes the hinge, CH2 and CH3, or the hinge, CH1, CH2 and CH3 regions of an IgG1 molecule. For the production of immunoglobulin fusions see also, U.S. Pat. No. 5,428,130.

### Preparation of a ZPA Polypeptide

The description below relates primarily to production of ZPA polypeptides by culturing cells transformed or transfected with a vector containing nucleic acid encoding ZPA polypeptides. It is, of course, contemplated that alternative methods that are known in the art can be employed to prepare a ZPA polypeptide. For instance, a ZPA polypeptide sequence, or portions thereof, can be produced by direct peptide synthesis using solid-phase techniques. See, e.g., Stewart et al., Solid-Phase Peptide Synthesis (W.H. Freeman Co.: San Francisco, Calif., 1969); Merrifield, J. Am. Chem. Soc., 85: 2149-2154 (1963). In vitro protein synthesis can be performed using manual techniques or by automation. Automated synthesis can be accomplished, for instance, with an Applied Biosystems Peptide Synthesizer (Foster City, Calif.) using manufacturer's instructions. Various portions of a ZPA polypeptide can be chemically synthesized separately and combined using chemical or enzymatic methods to produce the full-length ZPA polypeptide.

### Selection and Transformation of Host Cells

Host cells are transfected or transformed with expression or cloning vectors described herein for ZPA polypeptide production and cultured in conventional nutrient media modified as appropriate for inducing promoters, selecting transformants, or amplifying the genes encoding the desired sequences. The culture conditions, such as media, temperature, pH, and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of transfection are known to the ordinarily skilled artisan, for example, CaPO₄ treatment and electroporation. Depending on the host cell used, transformation is performed using standard techniques appropriate to such cells. The calcium treatment employing calcium chloride, as described in Sambrook et al., supra, or electroporation is generally used for prokaryotes or other cells that contain substantial cell-wall barriers. Infection with Agrobacterium tumefaciens is used for transformation of certain plant cells, as described by Shaw et al., Gene, 23: 315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transformations have been described in U.S. Pat. No. 4,399,216. Transformations into yeast are typically carried out according to the method of Van Solingen et al., J. Bact., 130: 946 (1977) and Hsiao et al., Proc. Natl. Acad. Sci. (USA), 76: 3829 (1979). However, other methods for introducing DNA into cells, such as by nuclear microinjection, electroporation, bacterial protoplast fusion with intact cells, or polycations, e.g., polybrene or polyornithine, can also be used. For various techniques for transforming mammalian cells, see, Keown et al., Methods in Enzymology, 185: 527-537 (1990) and Mansour et al., Nature, 336: 348-352 (1988).

Suitable host cells for cloning or expressing the DNA in the vectors herein include prokaryote, yeast, or higher eukaryote cells. Suitable prokaryotes include, but are not limited to, eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325); and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis (e.g., B. licheniformis 41P disclosed in DD 266,710 published 12 Apr. 1989), Pseudomonas such as P. aeruginosa, and Streptomyces. These examples are illustrative rather than limiting. In one embodiment, strain W3110 is the host or parent host because it is a common host strain for recombinant DNA product fermentations. In certain embodiments, the host cell secretes minimal amounts of proteolytic enzymes. For example, strain W3110 can be modified to effect a genetic mutation in the genes encoding proteins endogenous to the host, with examples of such hosts including E. coli W3110 strain 1A2, which has the complete genotype tonA; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

In addition to prokaryotes, eukaryotic microbes such as filamentous fungi or yeast are suitable cloning or expression hosts for vectors encoding a ZPA polypeptide. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9: 968-975 (1991)) such as, e.g., K. lactis (MW98-8C, CBS683, CBS4574; Louvencourt et al., J. Bacteriol., 737 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8: 135 (1990)), K. thermotolerans, and K. marxianus; yarrowia (EP 402,226); Pichia pastoris (EP 183,070; Sreekrishna et al., J. Basic Microbiol., 28: 265-278 [1988]); Candida; Trichoderma reesia (EP 244,234); Neurospora crassa (Case et al., Proc. Natl. Acad. Sci. USA, 76: 5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112: 284-289 [1983]; Tilburn et al., Gene, 26: 205-221 [1983]; Yelton et al., Proc. Natl. Acad. Sci. USA, 81: 1470-1474 [1984]) and A. niger (Kelly and Hynes, EMBO J., 4: 475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts can be found in C. Anthony, The Biochemistry of Methylotrophs, 269 (1982).

Suitable host cells for the expression of nucleic acid encoding a ZPA polypeptide are derived from multicellular organisms. Examples of invertebrate cells include insect cells such as Drosophila S2 and Spodoptera Sf9, as well as plant cells. Examples of useful mammalian host cell lines include Chinese hamster ovary (CHO) and COS cells. More specific examples include monkey kidney CV1 line transformed by SV40 (COS-7, ATCC CRL 1651); human embryonic kidney line (293 or 293 cells subcloned for growth in suspension culture, Graham et al., J. Gen. Virol., 36: 59 (1977)); Chinese hamster ovary cells/−DHFR(CHO, Urlaub and Chasin, Proc. Natl. Acad. Sci. USA, 77:4216 (1980)); mouse sertoli cells (TM4, Mather, Biol. Reprod., 23:243-251 (1980)); human lung cells (W138, ATCC CCL 75); human liver cells (Hep G2, HB 8065); and mouse mammary tumor (MMT 060562, ATCC CCL51). The selection of the appropriate host cell is deemed to be within the skill in the art.

### Selection and Use of a Replicable Vector

The nucleic acid (e.g., cDNA or genomic DNA) encoding a polypeptide or antibody of this invention can be inserted into a replicable vector for cloning (amplification of the DNA) or for expression. Various vectors are publicly available. The vector can, for example, be in the form of a plasmid, cosmid, viral particle, or phage. The appropriate nucleic acid sequence can be inserted into the vector by a variety of procedures. In general, DNA is inserted into an appropriate restriction endonuclease site(s) using techniques known in the art. Vector components generally include, but are not limited to, one or more of a signal sequence if the sequence is to be secreted, an origin of replication, one or more marker genes, an enhancer element, a promoter, and a transcription termination sequence. Construction of suitable vectors containing one or more of these components employs standard ligation techniques that are known to the skilled artisan.

The polypeptide or antibody of this invention can be produced recombinantly not only directly, but also as a fusion polypeptide with a heterologous polypeptide, which can be a signal sequence or other polypeptide having a specific cleavage site at the N-terminus of the mature protein or polypeptide. In general, the signal sequence can be a component of the vector, or it can be a part of the DNA encoding the polypeptide or antibody that is inserted into the vector. The signal sequence can be a prokaryotic signal sequence selected, for example, from the group of the alkaline phosphatase, penicillinase, lpp, or heat-stable enterotoxin II leaders. For yeast secretion the signal sequence can be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences can be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are known for a variety of bacteria, yeast, and viruses. The origin of replication from the plasmid pBR322 is suitable for most Gram-negative bacteria, the 2 [symbol] plasmid origin is suitable for yeast, and various viral origins (SV40, polyoma, adenovirus, VSV, or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up the nucleic acid encoding the polypeptide or antibody such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77: 4216 (1980). A suitable selection gene for use in yeast is the trp1 gene present in the yeast plasmid YRp7. Stinchcomb et al., Nature, 282: 39 (1979); Kingsman et al., Gene, 7: 141 (1979); Tschemper et al., Gene, 10: 157 (1980). The trp1 gene provides a selection marker for a mutant strain of yeast lacking the ability to grow in tryptophan, for example, ATCC No. 44076 or PEP4-1. Jones, Genetics, 85: 12 (1977).

Expression and cloning vectors usually contain a promoter operably linked to the nucleic acid sequence encoding the polypeptide or antibody of this invention to direct mRNA synthesis. Promoters recognized by a variety of potential host cells are known. Promoters suitable for use with prokaryotic hosts include the β-lactamase and lactose promoter systems (Chang et al., Nature, 275: 615 (1978); Goeddel et al., Nature, 281: 544 (1979)), alkaline phosphatase, a tryptophan (trp) promoter system (Goeddel, Nucleic Acids Res., 8: 4057 (1980); EP 36,776), and hybrid promoters such as the tac promoter (deBoer et al., Proc. Natl. Acad. Sci. USA, 80: 21-25 (1983)). Promoters for use in bacterial systems also will contain a Shine-Dalgarno (S.D.) sequence operably linked to the DNA encoding the polypeptide or antibody of this invention.

Examples of suitable promoting sequences for use with yeast hosts include the promoters for 3-phosphoglycerate kinase (Hitzeman et al., J. Biol. Chem., 255: 2073 (1980)) or other glycolytic enzymes (Hess et al., J. Adv. Enzyme Reg., 7: 149 (1968); Holland, Biochemistry, 17: 4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters that are inducible promoters having the additional advantage of transcription controlled by growth conditions are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Nucleic acid transcription from vectors in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus, and Simian Virus 40 (SV40); by heterologous mammalian promoters, e.g., the actin promoter or an immunoglobulin promoter; and by heat-shock promoters, provided such promoters are compatible with the host cell systems.

Transcription of a DNA encoding a polypeptide or antibody of this invention by higher eukaryotes can be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer can be spliced into the vector at a position 5′ or 3′ to the sequence coding for a polypeptide or antibody of this invention, but is preferably located at a site 5′ from the promoter. Expression vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding polypeptide or antibody.

Still other methods, vectors, and host cells suitable for adaptation to the synthesis of the polypeptide or antibody of this invention in recombinant vertebrate cell culture are described in Gething et al., Nature, 293: 620-625 (1981); Mantei et al., Nature, 281: 40-46 (1979); EP 117,060; and EP 117,058.

### Detecting Gene Amplification/Expression

Gene amplification and/or expression can be measured in a sample directly, for example, by conventional Southern blotting, Northern blotting to quantitate the transcription of mRNA (Thomas, Proc. Natl. Acad. Sci. USA, 77:5201-5205 (1980)), dot blotting (DNA analysis), or in situ hybridization, using an appropriately labeled probe, based on the sequences provided herein. Alternatively, antibodies can be employed that can recognize specific duplexes, including DNA duplexes, RNA duplexes, and DNA-RNA hybrid duplexes or DNA-protein duplexes. The antibodies in turn can be labeled and the assay can be carried out where the duplex is bound to a surface, so that upon the formation of duplex on the surface, the presence of antibody bound to the duplex can be detected.

Gene expression, alternatively, can be measured by immunological methods, such as immunohistochemical staining of cells or tissue sections and assay of cell culture or body fluids, to quantitate directly the expression of gene product. Antibodies useful for immunohistochemical staining and/or assay of sample fluids can be either monoclonal or polyclonal, and can be prepared in any mammal or can be synthesized (e.g., the monoclonal antibodies of this invention). Conveniently, the antibodies can be prepared against a native-sequence ZPA polypeptide or against a synthetic peptide based on the DNA sequences provided herein or against exogenous sequence fused to DNA encoding a ZPA polypeptide and encoding a specific antibody epitope.

Purification of ZPA polypeptides

Forms of ZPA polypeptides can be recovered from culture medium or from host cell lysates. If membrane-bound, it can be released from the membrane using a suitable detergent solution (e.g., TRITON-X™ 100) or by enzymatic cleavage. Cells employed in expression of nucleic acid encoding a ZPA polypeptide can be disrupted by various physical or chemical means, such as freeze-thaw cycling, sonication, mechanical disruption, or cell-lysing agents. According to one embodiment, it is desirable that a ZPA polypeptide is purified from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the ZPA polypeptide. Various methods of protein purification can be employed and such methods are known in the art and described, for example, in Deutscher, Methods in Enzymology, 182 (1990); Scopes, Protein Purification: Principles and Practice (Springer-Verlag: New York, 1982). The purification step(s) selected will depend, for example, on the nature of the production process used and the particular ZPA polypeptide produced. According to one embodiment, a ZPA polypeptide is purified by affinity chromatography using an antibody of this invention.

### Assaying Inhibition of Cell Proliferation

The inhibitory activity of one or more ZPA polypeptides and/or agonists of this invention on cell growth and proliferation can be measured using the assays described herein and other assays known in the art.

Animal models of tumors and cancers (e.g., breast cancer, colon cancer, prostate cancer, lung cancer, etc.) include both non-recombinant and recombinant (transgenic) animals. Non-recombinant animal models include, for example, rodent, e.g., murine models. Such models can be generated by introducing tumor cells into syngeneic mice using standard techniques, e.g., subcutaneous injection, tail vein injection, spleen implantation, intraperitoneal implantation, implantation under the renal capsule, or orthopin implantation, e.g., colon cancer cells implanted in colonic tissue. See, e.g., PCT publication No. WO 97/33551, published Sep. 18, 1997. Probably the most often used animal species in oncological studies are immunodeficient mice and, in particular, nude mice. The observation that the nude mouse with thymic hypo/aplasia could successfully act as a host for human tumor xenografts has led to its widespread use for this purpose. The autosomal recessive nu gene has been introduced into a very large number of distinct congenic strains of nude mouse, including, for example, ASW, A/He, AKR, BALB/c, B10.LP, C17, C3H, C57BL, C57, CBA, DBA, DDD, I/st, NC, NFR, NFS, NFS/N, NZB, NZC, NZW, P, RIII, and SJL. In addition, a wide variety of other animals with inherited immunological defects other than the nude mouse have been bred and used as recipients of tumor xenografts. For further details see, e.g., The Nude Mouse in Oncology Research, E. Boven and B. Winograd, eds. (CRC Press, Inc., 1991).

The cells introduced into such animals can be derived from known tumor/cancer cell lines, such as any of the above-listed tumor cell lines, and, for example, the B104-1-1 cell line (stable NIH-3T3 cell line transfected with the neu protooncogene); ras-transfected NIH-3T3 cells; Caco-2 (ATCC HTB-37); or a moderately well-differentiated grade TT human colon adenocarcinoma cell line, HT-29 (ATCC HTB-38); or from tumors and cancers. Samples of tumor or cancer cells can be obtained from patients undergoing surgery, using standard conditions involving freezing and storing in liquid nitrogen. Karmali et al., Br. J. Cancer, 48: 689-696 (1983).

Tumor cells can be introduced into animals such as nude mice by a variety of procedures. The subcutaneous (s.c.) space in mice is very suitable for tumor implantation. Tumors can be transplanted s.c. as solid blocks, as needle biopsies by use of a trochar, or as cell suspensions. For solid-block or trochar implantation, tumor tissue fragments of suitable size are introduced into the s.c. space. Cell suspensions are freshly prepared from primary tumors or stable tumor cell lines, and injected subcutaneously. Tumor cells can also be injected as subdermal implants. In this location, the inoculum is deposited between the lower part of the dermal connective tissue and the s.c. tissue.

Animal models of breast cancer can be generated, for example, by implanting rat neuroblastoma cells (from which the neu oncogene was initially isolated), or neu-transformed NIH-3T3 cells into nude mice, essentially as described by Drebin et al. Proc. Nat. Acad. Sci. USA, 83: 9129-9133 (1986).

Similarly, animal models of colon cancer can be generated by passaging colon cancer cells in animals, e.g., nude mice, leading to the appearance of tumors in these animals. An orthotopic transplant model of human colon cancer in nude mice has been described, for example, by Wang et al., Cancer Research, 54: 4726-4728 (1994) and Too et al., Cancer Research, 55: 681-684 (1995). This model is based on the so-called “METAMOUSE™” sold by AntiCancer, Inc., (San Diego, Calif.).

Tumors that arise in animals can be removed and cultured in vitro. Cells from the in vitro cultures can then be passaged to animals. Such tumors can serve as targets for further testing or drug screening. Alternatively, the tumors resulting from the passage can be isolated and RNA from pre-passage cells and cells isolated after one or more rounds of passage analyzed for differential expression of genes of interest. Such passaging techniques can be performed with any known tumor or cancer cell lines.

For example, Meth A, CMS4, CMS5, CMS21, and WEHI-164 are chemically induced fibrosarcomas of BALB/c female mice (DeLeo et al., J. Exp. Med., 146: 720 (1977)), which provide a highly controllable model system for studying the anti-tumor activities of various agents. Palladino et al., J. Immunol., 138: 4023-4032 (1987). Briefly, tumor cells are propagated in vitro in cell culture. Prior to injection into the animals, the cell lines are washed and suspended in buffer, at a cell density of about 10×10⁶ to 10×10⁷ cells/ml. The animals are then infected subcutaneously with 10 to 100 μl of the cell suspension, allowing one to three weeks for a tumor to appear.

In addition, the Lewis lung (3LL) carcinoma of mice, which is one of the most thoroughly studied experimental tumors, can be used as an investigational tumor model. Efficacy in this tumor model has been correlated with beneficial effects in the treatment of human patients diagnosed with small-cell carcinoma of the lung (SCCL). This tumor can be introduced in normal mice upon injection of tumor fragments from an affected mouse or of cells maintained in culture. Zupi et al., Br. J. Cancer, 41: suppl. 4, 30 (1980). Evidence indicates that tumors can be started from injection of even a single cell and that a very high proportion of infected tumor cells survive. For further information about this tumor model see, Zacharski, Haemostasis, 16: 300-320 (1986).

One way of evaluating the efficacy of a test compound in an animal model with an implanted tumor is to measure the size of the tumor before and after treatment. Traditionally, the size of implanted tumors has been measured with a slide caliper in two or three dimensions. The measure limited to two dimensions does not accurately reflect the size of the tumor; therefore, it is usually converted into the corresponding volume by using a mathematical formula. However, the measurement of tumor size is very inaccurate. The therapeutic effects of a drug candidate can be better described as treatment-induced growth delay and specific growth delay. Another important variable in the description of tumor growth is the tumor volume doubling time. Computer programs for the calculation and description of tumor growth are also available, such as the program reported by Rygaard and Spang-Thomsen, Proc. 6th Int. Workshop on Immune-Deficient Animals, Wu and Sheng eds. (Basel, 1989), p. 301. It is noted, however, that necrosis and inflammatory responses following treatment can actually result in an increase in tumor size, at least initially. Therefore, these changes need to be carefully monitored, by a combination of a morphometric method and flow cytometric analysis.

Further, recombinant (transgenic) animal models can be engineered by introducing the coding portion of a ZPA gene identified herein into the genome of animals of interest, using standard techniques for producing transgenic animals. Animals that can serve as a target for transgenic manipulation include, without limitation, mice, rats, rabbits, guinea pigs, sheep, goats, pigs, zebrafish, and non-human primates, e.g., baboons, chimpanzees and monkeys. Techniques known in the art to introduce a transgene into such animals include pronucleic microinjection (U.S. Pat. No. 4,873,191); retrovirus-mediated gene transfer into germ lines (e.g., Van der Putten et al., Proc. Natl. Acad. Sci. USA, 82: 6148-615 (1985)); gene targeting in embryonic stem cells (Thompson et al., Cell, 56: 313-321 (1989)); electroporation of embryos (Lo, Mol. Cell. Biol., 3: 1803-1814 (1983)); and sperm-mediated gene transfer. Lavitrano et al., Cell, 57: 717-73 (1989). For a review, see for example, U.S. Pat. No. 4,736,866.

For the purpose of the present invention, transgenic animals include those that carry the transgene only in part of their cells (“mosaic animals”). The transgene can be integrated either as a single transgene, or in concatamers, e.g., head-to-head or head-to-tail tandems. Selective introduction of a transgene into a particular cell type is also possible by following, for example, the technique of Lasko et al., Proc. Natl. Acad. Sci. USA, 89: 6232-636 (1992). The expression of the transgene in transgenic animals can be monitored by standard techniques. For example, Southern blot analysis or PCR amplification can be used to verify the integration of the transgene. The level of mRNA expression can then be analyzed using techniques such as in situ hybridization, Northern blot analysis, PCR, or immunocytochemistry. The animals are further examined for signs of tumor or cancer development.

Alternatively, “knock-out” animals, e.g., zebrafish, can be constructed that have a defective or altered gene encoding a ZPA polypeptide identified herein, as a result of homologous recombination between an endogenous gene encoding a ZPA polypeptide and altered genomic DNA encoding the same polypeptide introduced into an embryonic cell of the animal. For example, cDNA encoding a particular ZPA polypeptide can be used to clone genomic DNA encoding that polypeptide in accordance with established techniques. A portion of the genomic DNA encoding a particular ZPA polypeptide can be deleted or replaced with another gene, such as a gene encoding a selectable marker that can be used to monitor integration. Similarly, knock-out animals other than zebrafish can be constructed that have a defective or altered gene encoding an endogenous homolog of a ZPA polypeptide, as a result of homologous recombination between an endogenous gene encoding a ZPA homolog and altered genomic DNA encoding the homologous ZPA polypeptide introduced into an embryonic cell of the animal. Typically, several kilobases of unaltered flanking DNA (both at the 5′ and 3′ ends) are included in the vector. See, e.g., Thomas and Capecchi, Cell, 51: 503 (1987) for a description of homologous recombination vectors. The vector is introduced into an embryonic stem cell line (e.g., by electroporation) and cells in which the introduced DNA has homologously recombined with the endogenous DNA are selected. See, e.g., Li et al., Cell, 69: 915 (1992). The selected cells are then injected into a blastocyst of an animal (e.g., a mouse or rat) to form aggregation chimeras. See, e.g., Bradley, in Teratocarcinomas and Embryonic Stem Cells: A Practical Approach, E. J. Robertson, ed. (IRL: Oxford, 1987), pp. 113-152. A chimeric embryo can then be implanted into a suitable pseudopregnant female foster animal and the embryo brought to term to create a “knock-out” animal Progeny harboring the homologously recombined DNA in their germ cells can be identified by standard techniques and used to breed animals in which all cells of the animal contain the homologously recombined DNA. Knockout animals can be characterized, for instance, by their ability to defend against certain pathological conditions and by their development of pathological conditions due to absence of one or more ZPA polypeptides.

“Knock-down” animals (e.g., zebrafish), can be constructed in which the gene encoding a ZPA polypeptide is selectively prevented from being transcribed and/or translated. For example, silencing RNA or morpholinos may be used to block translation of one or more ZPA polypeptides. In such animals, the gene encoding a ZPA polypeptide remains intact, but the protein encoded by that gene is not produced.

The efficacy of antibodies specifically binding a ZPA polypeptide identified herein, and other drug candidates, can be tested also in the treatment of spontaneous animal tumors. A suitable target for such studies is the feline oral squamous cell carcinoma (SCC). Feline oral SCC is a highly invasive, malignant tumor that is the most common oral malignancy of cats, accounting for over 60% of the oral tumors reported in this species. It rarely metastasizes to distant sites, although this low incidence of metastasis can merely be a reflection of the short survival times for cats with this tumor. These tumors are usually not amenable to surgery, primarily because of the anatomy of the feline oral cavity. At present, there is no effective treatment for this tumor. Prior to entry into the study, each cat undergoes complete clinical examination and biopsy, and is scanned by computed tomography (CT). Cats diagnosed with sublingual oral squamous cell tumors are excluded from the study. The tongue can become paralyzed as a result of such tumor, and even if the treatment kills the tumor, the animals may not be able to feed themselves. Each cat is treated repeatedly, over a longer period of time. Photographs of the tumors will be taken daily during the treatment period, and at each subsequent recheck. After treatment, each cat undergoes another CT scan. CT scans and thoracic radiograms are evaluated every 8 weeks thereafter. The data are evaluated for differences in survival, response, and toxicity as compared to control groups. Positive response may require evidence of tumor regression, preferably with improvement of quality of life and/or increased life span.

In addition, other spontaneous animal tumors, such as fibrosarcoma, adenocarcinoma, lymphoma, chondroma, or leiomyosarcoma of dogs, cats, and baboons can also be tested. Of these, mammary adenocarcinoma in dogs and cats is a preferred model as its appearance and behavior are very similar to those in humans. However, the use of this model is limited by the rare occurrence of this type of tumor in animals.

### Construction of Transgenic Zebrafish

Transgenic zebrafish may be constructed as described herein, or as well known in the art (see, e.g., Westerfield, The Zebrafish Book. A guide for the laboratory use of zebrafish (Danio rerio). 4^{th} ed., Univ. of Oregon Press: Eugene (2000)). Transgenic constructs can be introduced into zebrafish cells (for example, at the 1-4 cell stage of development), and the injected embryos then be allowed to develop until such time as appropriate to examine the effects of the transgene. Transgenic constructs can be linear or circular polynucleotides, and optionally may include regulatory sequences as described elsewhere herein. Methods of introducing the trans genic construct into embryonic zebrafish cells include, but are not limited to, microinjection, electroporation, particle gun bombardment, viral infection, and via liposomes. A reporter molecule can be included in the transgenic construct for ease of determining the presence of the transgene in the adult zebrafish (e.g., GFP or some other readily identifiable label); in situations where no reporter was included in the construct, the zebrafish nucleic acid (e.g., isolated from a tail cutting from an adult zebrafish) can be examined for the presence of the transgene by well-known genetic methods such as PCR or southern blotting.

Any strain and/or variety of laboratory or commercially available zebrafish may be used in the methodologies described herein. In some embodiments, zebrafish are from inbred lines (including, but not limited to, SJD, C32, and WIK). In some embodiments, visualization of transgene activity is facilitated by the use of zebrafish having other mutations, for example “non-pigmented” mutant zebrafish substantially devoid of melanophore deposition (e.g. albino mutants) or irridiphore deposition (e.g. roy orbison, transparent mutants).

### Assays for Evaluating Apoptotic Activity

Assays that are useful for measuring the pro-apoptotic or anti-apoptotic activity of the agonists, progenitors, and antagonists of this invention include the assays of Example 3 or other suitable assays known in the art such as those included below.

Assays for apoptotic activity include, for example, cytotoxicity assays (e.g., radiometric or non-radiometric assays measuring increased membrane permeability or colorimetric assays measuring reduction in the metabolic activity of mitochondria); assays measuring DNA fragmentation (including, but not limited to, in situ nick translation (ISNT) and TdT-mediated X-dUTP nick end labeling (TUNEL) (Cole and Ross, Devel. Biol. 240: 123-142 (2001))); assays measuring changes in cellular organization and packaging which are precursors to cell death (e.g., alterations in membrane asymmetry (including, but not limited to, translocation of phosphatidylserine (Nicoletti et al., “Common Methods for Measuring Apoptotic Cell Death by Flow Cytometry,” The Purdue Cytometry CD-ROM Volume 3, J. Parker, C. Stewart, Guest Eds., J. Paul Robinson, Publisher. Purdue University, West Lafayette, 1997, ISBN 1-890473-02-2), and release of cytochrome C or AIF from the mitochondria into the cytoplasm); and assays measuring activation of one or more biochemical cascades resulting in apoptosis (including, but not limited to, caspase activation (see, e.g., Example 3), and cleavage of poly-ADP-ribose polymerase (PARP)). Assays for apoptotic activity can be performed on single cells and/or on cellular populations.

### Antibody Binding Studies

Antibody binding studies can be carried out using known assay methods, such as competitive binding assays, direct and indirect sandwich assays, and immunoprecipitation assays. Zola, Monoclonal Antibodies: A Manual of Techniques (CRC Press, Inc., 1987), pp. 147-158.

Competitive binding assays rely on the ability of a labeled standard to compete with the test sample analyte for binding with a limited amount of antibody. The amount of target protein in the test sample is inversely proportional to the amount of standard that becomes bound to the antibodies. To facilitate determining the amount of standard that becomes bound, the antibodies can be insolubilized before or after the competition, so that the standard and analyte that are bound to the antibodies can conveniently be separated from the standard and analyte that remain unbound.

Sandwich assays involve the use of two antibodies, each capable of binding to a different immunogenic portion, or epitope, of the protein to be detected. In a sandwich assay, the test sample analyte is bound by a first antibody that is immobilized on a solid support, and thereafter a second antibody binds to the analyte, thus forming an insoluble three-part complex. See, e.g., U.S. Pat. No. 4,376,110. The second antibody can itself be labeled with a detectable moiety (direct sandwich assays) or can be measured using an anti-immunoglobulin antibody that is labeled with a detectable moiety (indirect sandwich assay). For example, one type of sandwich assay is an ELISA assay, in which case the detectable moiety is an enzyme.

Competitive ELISA assays can be performed to screen polypeptides, agonists or antagonists for those that specifically bind to a ZPA polypeptide, which binding can be inhibited by a monoclonal antibody of this invention.

In one example, a competitive ELISA assay can be conducted following methods known in the art. A full length or truncated form of a native ZPA protein (2 ug/ml in PBS) can be coated on a microtiter plate at 4° C. overnight or at room temperature for 2 hours. The wells can be blocked by adding 65 μl 1% BSA for 30 minutes followed by 40 ul 1% Tween 20 for another 30 minutes. Next, the wells can be washed with PBS-0.05% Tween 20 5 times. Various concentrations of antibody (in ELISA buffer) can be incubated in the wells for 30 minutes at room temperature. Then, polypeptides or antibodies to be tested can be added to different wells for 10 minutes at a concentration that would normally produce 90% binding capacity in the absence of the antibody. Next, the wells can be washed with PBS-0.05% Tween 20 5 times. Binding can be quantified by methods known in the art.

For immunohistochemistry, the tissue sample can be fresh or frozen or can be embedded in paraffin and fixed with a preservative such as formalin, for example.

### Cell-Based Tumor Assays

Cell-based assays and animal models for proliferative disorders, such as tumors, can be used to verify the inhibitory activity of the antagonists of this invention. Appropriate assays are known in the art. For example, cells of a cell type known to be involved in a proliferative disorder can be transfected with one or more ZPA cDNAs herein, and the ability of these cDNAs to inhibit growth is analyzed in the presence or absence of an antagonist. If the proliferative disorder is cancer, suitable tumor cells include, for example, stable tumor cell lines such as the B104-1-1 cell line (stable NTH-3T3 cell line transfected with the neu protooncogene) and ras-transfected NIH-3T3 cells, which can be transfected with a ZPA sequence and monitored for tumorigenic growth. Such transfected cell lines can then be used to test the ability of poly- or monoclonal antibodies or antibody compositions to inhibit tumorigenic cell growth by exerting cytostatic or cytotoxic activity on the growth of the transformed cells, or by mediating antibody-dependent cellular cytotoxicity (ADCC).

In addition, primary cultures derived from tumors in transgenic animals (as described above) can be used in the cell-based assays herein, although stable cell lines are preferred. Techniques to derive continuous cell lines from transgenic animals are known in the art. See, e.g., Small et al., Mol. Cell. Biol., 5: 642-648 (1985).

### Gene Therapy

Described below are methods and compositions whereby disease symptoms can be ameliorated. The ZPA polypeptides (including ZPA polypeptide variants) described herein, and antagonists and antibodies of this invention can be employed in accordance with the present invention by expression of each in vivo, which is often referred to as gene therapy. For example, ZPA polypeptide variants can be expressed in cells using these methods. According to one embodiment, the methods or the vectors used to express the ZPA polypeptides (including variants) involve the use of a targeting agent to direct the vehicle containing the ZPA polypeptide or nucleic acid molecule to a desired tissue.

There are two major approaches to getting the nucleic acid (optionally contained in a vector) into the mammal's cells: in vivo and ex vivo. For in vivo delivery the nucleic acid is injected directly into the mammal, usually at the sites where the ZPA polypeptide is required, i.e., the site of synthesis of the ZPA polypeptide, if known, and the site (e.g., wound) where biological activity of the ZPA polypeptide is needed. For ex vivo treatment, the mammal's cells are removed, the nucleic acid is introduced into these isolated cells, and the modified cells are administered to the mammal either directly or, for example, encapsulated within porous membranes that are implanted into the mammal (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or transferred in vivo in the cells of the intended host. Techniques suitable for the transfer of nucleic acid into mammalian cells in vitro include the use of liposomes, electroporation, microinjection, transduction, cell fusion, DEAE-dextran, the calcium phosphate precipitation method, etc. Transduction involves the association of a replication-defective, recombinant viral (including, but not limited to, retroviral) particle with a cellular receptor, followed by introduction of the nucleic acids contained by the particle into the cell. A commonly used vector for ex vivo delivery of the gene is a retrovirus.

Commonly used in vivo nucleic acid transfer techniques include transfection with viral or non-viral vectors (such as adenovirus, lentivirus, Herpes simplex I virus, or adeno-associated virus (AAV)) and lipid-based systems (useful lipids for lipid-mediated transfer of the gene are, for example, DOTMA, DOPE, and DC-Chol; see, e.g., Tonkinson et al., Cancer Investigation, 14(1): 54-65 (1996)). Such vectors are used to synthesize virus that can be used as vehicles for delivering agents, such as antagonists and nucleic acid molecules of this invention. The most commonly used vectors for use in gene therapy are viruses, e.g., adenoviruses, AAV, lentiviruses, or retroviruses. A viral vector such as a retroviral vector includes at least one transcriptional promoter/enhancer or locus-defining element(s), or other elements that control gene expression by other means such as alternate splicing, nuclear RNA export, or post-translational modification of messenger. In addition, a viral vector such as a retroviral vector includes a nucleic acid molecule that, when transcribed in the presence of a gene encoding a ZPA polypeptide, is operably linked thereto and acts as a translation initiation sequence. Such vector constructs also include a packaging signal, long terminal repeats (LTRs) or portions thereof, and positive and negative strand primer binding sites appropriate to the virus used (if these are not already present in the viral vector). In addition, such vector typically includes a signal sequence for secretion of the ZPA polypeptide from a host cell in which it is placed. In certain embodiments, the signal sequence for this purpose is a mammalian signal sequence, including, but not limited to, the native signal sequence for the ZPA polypeptide. Optionally, the vector construct can also include a signal that directs polyadenylation, as well as one or more restriction sites and a translation termination sequence. By way of example, such vectors will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

In some situations, it is desirable to provide the nucleic acid source with an agent that targets the target cells, such as an antibody specific for a cell-surface membrane protein or the target cell, a ligand for a receptor on the target cell, etc. Where liposomes are employed, proteins that bind to a cell-surface membrane protein associated with endocytosis can be used for targeting and/or to facilitate uptake, e.g., capsid proteins or fragments thereof tropic for a particular cell type, antibodies for proteins that undergo internalization in cycling, and proteins that target intracellular localization and enhance intracellular half-life. The technique of receptor-mediated endocytosis is described, for example, by Wu et al., J. Biol. Chem., 262: 4429-4432 (1987); and Wagner et al., Proc. Natl. Acad. Sci. USA, 87: 3410-3414 (1990). For a review of the currently known gene marking and gene therapy protocols, see, Anderson et al., Science, 256: 808-813 (1992). See also WO 93/25673 and the references cited therein.

Suitable gene therapy and methods for making retroviral particles and structural proteins can be found in, e.g., U.S. Pat. No. 5,681,746.

### Detecting ZPA Mutations

This invention is also related to the use of the gene encoding a ZPA polypeptide as a diagnostic. Detection of a mutated form of a ZPA polypeptide can be indicative of a proclivity for developing an apoptosis-related disorder. Detection of levels of the ZPA polypeptide in the tissue of a zebrafish over the levels of the same tissue in a normal zebrafish can also be indicative of a proclivity for developing an apoptosis-related disorder. Similarly, detection of a mutated form of a homolog of a ZPA polypeptide in an organism other than zebrafish can be indicative of a proclivity for developing an apoptosis-related disorder. Detection of levels of a homolog of a ZPA polypeptide in the tissue of an organism (e.g., a mammal), over the levels of the same tissue in a normal organism can also be indicative of a proclivity for developing an apoptosis-related disorder.

Individuals carrying mutations in the genes encoding a human homolog of a ZPA polypeptide can be detected at the DNA level by a variety of techniques. Nucleic acids for diagnosis can be obtained from a mammal's cells, such as from blood, urine, saliva, tissue biopsy, and autopsy material. The genomic DNA can be used directly for detection or can be amplified enzymatically by using PCR (Saiki et al., Nature, 324: 163-166 (1986)) prior to analysis. RNA or cDNA can also be used for the same purpose. As an example, PCR primers complementary to the nucleic acid encoding a ZPA polypeptide can be used to identify and analyze mutations in the human homolog of a ZPA polypeptide. For example, deletions and insertions can be detected by a change in size of the amplified product in comparison to the normal genotype. Point mutations can be identified by hybridizing amplified DNA to radiolabeled RNA encoding a ZPA polypeptide, or alternatively, radiolabeled antisense DNA sequences encoding a ZPA polypeptide. Perfectly matched sequences can be distinguished from mismatched duplexes by RNase A digestion or by differences in melting temperatures.

Genetic testing based on DNA sequence differences can be achieved by detection of alteration in electrophoretic mobility of DNA fragments in gels with or without denaturing agents. Small sequence deletions and insertions can be visualized by high resolution gel electrophoresis. DNA fragments of different sequences can be distinguished on denaturing formamidine gradient gels in which the mobilities of different DNA fragments are retarded in the gel at different positions according to their specific melting or partial melting temperatures. See, e.g., Myers et al., Science, 230: 1242 (1985).

Sequence changes at specific locations may also be revealed by nuclease protection assays, such as RNase and S1 protection or the chemical cleavage method, for example, Cotton et al., Proc. Natl. Acad. Sci. USA, 85: 4397-4401 (1985).

Thus, the detection of a specific DNA sequence can be achieved by methods such as hybridization, RNase protection, chemical cleavage, direct DNA sequencing, or the use of restriction enzymes, e.g., restriction fragment length polymorphisms (RFLP), and Southern blotting of genomic DNA.

In addition to more conventional gel-electrophoresis and DNA sequencing, mutations in a ZPA polypeptide can also be detected by in situ analysis.

### Detecting ZPA Polypeptide or Nucleic Acid Levels

Levels of ZPA polypeptide or nucleic acid molecules can be detected, e.g., using the reagents disclosed herein in combination with methods known in the art, such as in situ hybridization, RT-PCR, northern blots, western blots, or by using the Examples and reagents provided herein. Similarly, levels of polypeptides or nucleic acid molecules homologous to a ZPA polypeptide or nucleic acid molecule can be detected, e.g., using the reagents disclosed herein in combination with methods known in the art, such as in situ hybridization, RT-PCR, northern blots, western blots, or by using the Examples and reagents provided herein.

A competition assay can be employed wherein antibodies specific to a ZPA polypeptide are attached to a solid support and a labeled ZPA polypeptide and a sample derived from the host comprising at least one polypeptide homologous to one or more ZPA polypeptides are passed over the solid support and the amount of label detected attached to the solid support can be correlated to a quantity of the at least one polypeptide homologous to one or more ZPA polypeptides in the sample. In one embodiment, antibodies that specifically bind a ZPA polypeptide as described herein are used to monitor ZPA polypeptide levels or levels of a ZPA polypeptide homolog.

### Screening Assays for Drug Candidates

This invention encompasses methods of screening compounds to identify those that mimic a ZPA polypeptide activity (agonists) or prevent the effect of a ZPA polypeptide (antagonists). Generally, a ZPA polypeptide is exposed to the drug candidate by incubation or contact under various conditions. Screening assays for antagonist drug candidates are designed to identify compounds that specifically bind or complex with a native ZPA polypeptide. Such screening assays will include assays amenable to high-throughput screening of chemical libraries, making them particularly suitable for identifying small molecule drug candidates.

The assays can be performed in a variety of formats, including protein-protein binding assays, biochemical screening assays, immunoassays, and cell-based assays in combination with a ZPA polypeptide, fragments thereof, or cells expressing a ZPA polypeptide or fragments thereof.

All assays for antagonists are common in that they call for contacting the drug candidate with a ZPA polypeptide encoded by a nucleic acid identified herein under conditions and for a time sufficient to allow these two components to interact.

In binding assays, the interaction is binding and the complex formed can be isolated or detected in the reaction mixture. For example, binding of a ZPA polypeptide to a polypeptide with which it normally interacts in one or more biochemical pathways in the absence or presence of the candidate antagonist can be performed to evaluate whether the antagonist blocked binding of the ZPA polypeptide to polypeptide with which it normally interacts. In another embodiment, a ZPA polypeptide encoded by a gene identified herein or the drug candidate is immobilized on a solid phase, e.g., on a microtiter plate, by covalent or non-covalent attachments. Non-covalent attachment generally is accomplished by coating the solid surface with a solution of the ZPA polypeptide and drying. Alternatively, an immobilized antibody, e.g., a monoclonal antibody, specific for a ZPA polypeptide to be immobilized can be used to anchor it to a solid surface. The assay is performed by adding the non-immobilized component, which can be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular ZPA polypeptide, its interaction with that polypeptide can be assayed by methods known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340: 245-246 (1989); Chien et al., Proc. Natl. Acad. Sci. USA, 88: 9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with binding between a ZPA polypeptide and another protein, including another ZPA polypeptide, can be tested as follows: usually a reaction mixture is prepared containing the ZPA polypeptide and the other protein under conditions and for a time allowing for the interaction and binding of the two proteins. To test the ability of a candidate compound to inhibit binding, the reaction is run in the absence and in the presence of the test compound. In addition, a placebo can be added to a third reaction mixture, to serve as positive control. The binding (complex formation) between the test compound and the other polypeptide present in the mixture is monitored as described hereinabove. The formation of a complex in the control reaction(s) but not in the reaction mixture containing the test compound indicates that the test compound interferes with the interaction of the ZPA polypeptide and the other polypeptide.

According to one embodiment, assays described herein are performed to test antagonists of this invention. Alternatively, antagonists can be detected by combining a ZPA polypeptide and a potential antagonist with unlabeled ZPA polypeptide under appropriate conditions for a competitive inhibition assay. The ZPA polypeptide can be labeled, such as by radioactivity or a colorimetric method, such that the number of ZPA polypeptide molecules bound can be used to determine the effectiveness of the potential antagonist. The ZPA polypeptide can be labeled by a variety of means including iodination or inclusion of a recognition site for a site-specific protein kinase. Following fixation and incubation, the slides are subjected to autoradiographic analysis

Drug candidates include anti-ZPA antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a drug candidate can be a closely related protein, for example, a mutated form of a ZPA polypeptide that competitively inhibits the action of the endogenous ZPA polypeptide or endogenous ZPA polypeptide homolog.

### Administration Protocols, Schedules, Doses, and Formulations

The molecules herein and antagonists thereto are pharmaceutically useful as a prophylactic and therapeutic agents for various disorders and diseases as set forth above.

Therapeutic compositions of the polypeptides, agonists or antagonists of this invention are prepared for storage by mixing the desired molecule having the appropriate degree of purity with optional pharmaceutically acceptable carriers, excipients, or stabilizers (Remington's Pharmaceutical Sciences, 16th edition, Osol, A. ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol. Carriers for topical or gel-based forms of agonist or antagonist include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations. The ZPA polypeptides or agonists or antagonists will typically be formulated in such vehicles at a concentration of about 0.1 mg/ml to 100 mg/ml.

Another formulation comprises incorporating a ZPA polypeptide or agonist or antagonist thereof into formed articles. Such articles can be used in modulating endothelial cell growth and angiogenesis. In addition, tumor invasion and metastasis can be modulated with these articles.

ZPA polypeptides or agonists or antagonists to be used for in vivo administration must be sterile. This is readily accomplished by filtration through sterile filtration membranes, prior to or following lyophilization and reconstitution. ZPA polypeptides ordinarily will be stored in lyophilized form or in solution if administered systemically. If in lyophilized form, a ZPA polypeptide or agonist or antagonist thereto is typically formulated in combination with other ingredients for reconstitution with an appropriate diluent at the time for use. An example of a liquid formulation of a ZPA polypeptide or agonist or antagonist is a sterile, clear, colorless, unpreserved solution filled in a single-dose vial for subcutaneous injection. Preserved pharmaceutical compositions suitable for repeated use can contain, for example, depending mainly on the indication and type of polypeptide:
a. a ZPA polypeptide or agonist or antagonist thereto;
b. a buffer capable of maintaining the pH in a range of maximum stability of the polypeptide or other molecule in solution, e.g., about pH 4-8;
c. a detergent/surfactant primarily to stabilize the polypeptide or molecule against agitation-induced aggregation;
d. an isotonifier;
e. a preservative selected from the group of phenol, benzyl alcohol and a benzethonium halide, e.g., chloride; and
f. water.

If the detergent employed is non-ionic, it can, for example, be polysorbates (e.g., POLYSORBATE™ (TWEEN™) 20, 80, etc.) or poloxamers (e.g., POLOXAMER™ 188). The use of non-ionic surfactants permits the formulation to be exposed to shear surface stresses without causing denaturation of the polypeptide. Further, such surfactant-containing formulations can be employed in aerosol devices such as those used in a pulmonary dosing, and needleless jet injector guns (see, e.g., EP 257,956).

An isotonifier can be present to ensure isotonicity of a liquid composition of a ZPA polypeptide or agonist or antagonist thereto, and includes polyhydric sugar alcohols, e.g., trihydric or higher sugar alcohols, such as glycerin, erythritol, arabitol, xylitol, sorbitol, and mannitol. These sugar alcohols can be used alone or in combination. Alternatively, sodium chloride or other appropriate inorganic salts can be used to render the solutions isotonic.

The buffer can, for example, be an acetate, citrate, succinate, or phosphate buffer depending on the pH desired. The pH of one type of liquid formulation of this invention is buffered in the range of about 4 to 8, preferably about physiological pH.

The preservatives phenol, benzyl alcohol and benzethonium halides, e.g., chloride, are known antimicrobial agents that can be employed.

Therapeutic ZPA polypeptide, agonist, and/or antagonist compositions generally are placed into a container having a sterile access port, for example, an intravenous solution bag or vial having a stopper pierceable by a hypodermic injection needle. In certain embodiments, the formulations can be administered as repeated intravenous (i.v.), subcutaneous (s.c.), or intramuscular (i.m.) injections, or as aerosol formulations suitable for intranasal or intrapulmonary delivery (for intrapulmonary delivery see, e.g., EP 257,956).

ZPA polypeptides, agonists and/or antagonists thereto can also be administered in the form of sustained-released preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot™ (injectable microspheres composed of lactic acid-glycolic acid copolymer and leuprolide acetate), and poly-D-(−)-3-hydroxybutyric acid (EP 133,988).

While polymers such as ethylene-vinyl acetate and lactic acid-glycolic acid enable release of molecules for over 100 days, certain hydrogels release proteins for shorter time periods. When encapsulated proteins remain in the body for a long time, they can denature or aggregate as a result of exposure to moisture at 37° C., resulting in a loss of biological activity and possible changes in immunogenicity. Rational strategies can be devised for protein stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

Sustained-release ZPA polypeptide, agonist, and/or antagonist compositions also include liposomally entrapped ZPA polypeptides, agonists, and/or antagonists. Liposomes containing a ZPA polypeptide, antibody, agonist, and/or antagonist are prepared by methods known per se: DE U.S. Pat. No. 3,218,121; Epstein et al., Proc. Natl. Acad. Sci. USA, 82: 3688-3692 (1985); Hwang et al., Proc. Natl. Acad. Sci. USA, 77: 4030-4034 (1980); EP 52,322; EP 36,676; EP 88,046; EP 143,949; EP 142,641; Japanese patent application 83-118008; U.S. Pat. Nos. 4,485,045 and 4,544,545; and EP 102,324. Ordinarily the liposomes are of the small (about 200-800 Angstroms) unilamellar type in which the lipid content is greater than about 30 mol. % cholesterol, the selected proportion being adjusted for the optimal therapy.

The therapeutically effective dose of a ZPA polypeptide, agonist, and/or antagonist thereto will, of course, vary depending on such factors as the disorder to be treated (including prevention), the method of administration, the type of compound being used for treatment, any co-therapy involved, the patient's age, weight, general medical condition, medical history, etc., and its determination is well within the skill of a practicing physician. Accordingly, it will be necessary for the therapist to titer the dosage and modify the route of administration as required to obtain the maximal therapeutic effect. The clinician will administer a ZPA polypeptide, antibody, agonist and/or antagonist thereto until a dosage is reached that achieves the desired effect for treatment of the condition in question. For example, if the objective is the treatment of cancer, the amount would be one that inhibits the growth of the cancer.

With the above guidelines, the effective dose generally is within the range of from about 0.001 to about 1.0 mg/kg, about 0.01-1.0 mg/kg, and/or about 0.01-0.1 mg/kg.

For non-oral use in treating apoptotic-related disorders, it is advantageous to administer the ZPA polypeptide, agonist and/or antagonist thereto in the form of an injection at about 0.01 to 50 mg, about 0.05 to 20 mg, and/or about 1 to 20 mg per kg body weight, 1 to 3 times daily by intravenous injection. For oral administration, in certain embodiments, a molecule based on a ZPA polypeptide, agonist, and/or antagonist is administered at about 5 mg to 1 g, and/or about 10 to 100 mg per kg body weight, 1 to 3 times daily. It should be appreciated that endotoxin contamination should be kept minimally at a safe level, for example, less than 0.5 ng/mg protein. Moreover, for human administration, the formulations preferably meet sterility, pyrogenicity, general safety, and purity as required by FDA Office and Biologics standards.

The dosage regimen of a pharmaceutical composition containing a ZPA polypeptide, agonist, and/or antagonist to be used in tissue regeneration will be determined by the attending physician considering various factors that modify the action of the polypeptides, e.g., amount of tissue weight desired to be formed, the site of damage, the condition of the damaged tissue, the size of a wound, type of damaged tissue (e.g., bone), the patient's age, sex, and diet, the severity of any infection, time of administration, and other clinical factors. The dosage can vary with the type of matrix used in the reconstitution and with inclusion of other proteins in the pharmaceutical composition. For example, the addition of other known growth factors, such as IGF-1, to the final composition can also affect the dosage. Progress can be monitored by periodic assessment of tissue/bone growth and/or repair, for example, X-rays, histomorphometric determinations, and tetracycline labeling.

The route of ZPA polypeptide, antagonist and/or agonist administration is in accord with known methods, e.g., by injection or infusion by intravenous, intramuscular, intracerebral, intraperitoneal, intracerobrospinal, subcutaneous, intraocular, intraarticular, intrasynovial, intrathecal, oral, topical, or inhalation routes, or by sustained-release systems as noted below. A ZPA polypeptide, agonist and/or antagonist thereof also are suitably administered by intratumoral, peritumoral, intralesional, or perilesional routes, to exert local as well as systemic therapeutic effects.

If a peptide or small molecule is employed as an antagonist or agonist, it is preferably administered orally or non-orally in the form of a liquid or solid to mammals.

Examples of pharmacologically acceptable salts of molecules that form salts and are useful hereunder include alkali metal salts (e.g., sodium salt, potassium salt), alkaline earth metal salts (e.g., calcium salt, magnesium salt), ammonium salts, organic base salts (e.g., pyridine salt, triethylamine salt), inorganic acid salts (e.g., hydrochloride, sulfate, nitrate), and salts of organic acid (e.g., acetate, oxalate, p-toluenesulfonate).

The location of the desired action of a ZPA polypeptide, agonist, and/or antagonist thereto of the invention may be taken into consideration in preparation and administration of the polypeptide, agonist, and/or antagonist. When the desired action is located intracellularly, certain embodiments of the invention provide for the polypeptide, agonist, and/or antagonist to be introduced into the cell. In one embodiment, intracellular expression of a polypeptide, proteinaceous agonist, and/or proteinaceous antagonist is effected by introducing a nucleic acid encoding the polypeptide, proteinaceous agonist, and/or proteinaceous antagonist (lacking the wild-type leader sequence and secretory signals normally associated with the gene encoding such molecules) into a target cell. Any standard method of introducing nucleic acids into a cell may be used, including, but not limited to, microinjection, ballistic injection, electroporation, calcium phosphate precipitation, liposomes, and transfection with retroviral, adenoviral, adeno-associated viral and vaccinia vectors carrying the nucleic acid of interest.

In another embodiment, internalizing molecules are provided. Polypeptides can possess certain characteristics that enhance their delivery into cells, or can be modified to possess such characteristics. Techniques for achieving this are known in the art. For example, cationization, lipofections or liposomes can be used to deliver the antibody into cells. Translocation of molecules into cells can also be facilitated by conjugating a pH (low) insertion peptide (“pHLIP”) to the molecule to be translocated (e.g., by disulfide bonds, see, for example, Reshetnyak et al., Proc. Natl. Acad. Sci. 103(17): 6460-6465 (2006)) and lowering the extracellular pH. Where fragments are used, the smallest fragment that performs the desired function is generally advantageous. For example, a ZPA polypeptide lacking a membrane anchor region while retaining anti-apoptotic activity may be advantageous for intracellular introduction over the wild-type polypeptide. Such peptides can be synthesized chemically and/or produced by recombinant DNA technology.

Entry of modulator polypeptides, agonists, and antagonists into target cells can be enhanced by methods known in the art. For example, certain sequences, such as those derived from HIV Tat or the Antennapedia homeodomain protein are able to direct efficient uptake of heterologous proteins across cell membranes. See, e.g., Chen et al., Proc. Natl. Acad. Sci. USA (1999), 96:4325-4329.

When the location of desired activity of a ZPA polypeptide, agonist, and/or antagonist is in the brain, certain embodiments of the invention provide for the ZPA polypeptide, agonist, and/or antagonist to traverse the blood-brain barrier. Certain neurodegenerative diseases are associated with an increase in permeability of the blood-brain barrier, such that the antibody or antigen-binding fragment can be readily introduced to the brain. When the blood-brain barrier remains intact, several art-known approaches exist for transporting molecules across it, including, but not limited to, physical methods, lipid-based methods, and receptor and channel-based methods.

Physical methods of transporting the ZPA polypeptide, agonist, and/or antagonist across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9: 398-406 (2002)), interstitial infusion/convection-enhanced delivery (see, e.g., Bobo et al., Proc. Natl. Acad. Sci. USA 91: 2076-2080 (1994)), and implanting a delivery device in the brain (see, e.g., Gill et al., Nature Med. 9: 589-595 (2003); and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the ZPA polypeptide, proteinaceous agonist, and/or proteinaceous antagonist (see, e.g., U.S. Patent Publication No. 2003/0083299).

Lipid-based methods of transporting a ZPA polypeptide, agonist, and/or antagonist across the blood-brain barrier include, but are not limited to, encapsulating the ZPA polypeptide, agonist, and/or antagonist in liposomes that are coupled to antibody binding fragments that bind to receptors on the vascular endothelium of the blood-brain barrier (see, e.g., U.S. Patent Application Publication No. 20020025313), and coating the ZPA polypeptide, agonist, and/or antagonist in low-density lipoprotein particles (see, e.g., U.S. Patent Application Publication No. 20040204354) or apolipoprotein E (see, e.g., U.S. Patent Application Publication No. 20040131692).

Receptor and channel-based methods of transporting the ZPA polypeptide, agonist, and/or antagonist across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating the molecules with a transferrin and modulating activity of the one or more transferrin receptors (see, e.g., U.S. Patent Application Publication No. 2003/0129186), and cationizing the molecules (see, e.g., U.S. Pat. No. 5,004,697).

### Combination Therapies

The effectiveness of a ZPA polypeptide or an agonist or antagonist thereof in preventing or treating an apoptosis-related disorder can be improved by administering the active agent serially or in combination with another agent that is effective for those purposes, either in the same composition or as separate compositions.

For example, for treatment of cell proliferative disorders, ZPA polypeptide and/or ZPA polypeptide agonist therapy can be combined with the administration of other inhibitors of cell proliferation, such as cytotoxic agents.

In addition, ZPA polypeptides and/or agonists used to treat cancer can be combined with cytotoxic, chemotherapeutic, or growth-inhibitory agents as identified above. Also, for cancer treatment, a ZPA polypeptide and/or agonist thereof is suitably administered serially or in combination with radiological treatments, whether involving irradiation or administration of radioactive substances.

If the treatment is for cancer, it may be desirable also to administer antibodies against tumor-associated antigens, such as antibodies that bind to one or more of the ErbB2, EGFR, ErbB3, ErbB4, or VEGF receptor(s). Alternatively, or in addition, two or more antibodies binding the same or two or more different antigens disclosed herein may be co-administered to the patient. Sometimes, it may be beneficial also to administer one or more cytokines to the patient. In one embodiment, a ZPA polypeptide and/or agonist thereof described herein are co-administered with a growth-inhibitory agent. For example, the growth-inhibitory agent may be administered first, followed by a ZPA polypeptide and/or agonist thereof of the present invention. However, simultaneous administration or administration of a ZPA polypeptide and/or agonist thereof of the present invention first is also contemplated. Suitable dosages for the growth-inhibitory agent are those presently used and may be lowered due to the combined action (synergy) of the growth-inhibitory agent and the polypeptides and agonists described herein.

In one embodiment, vascularization of tumors is attacked in combination therapy. A ZPA polypeptide and/or agonist thereof of this invention and an antibody (e.g., anti-VEGF) are administered to tumor-bearing patients at therapeutically effective doses as determined, for example, by observing necrosis of the tumor or its metastatic foci, if any. This therapy is continued until such time as no further beneficial effect is observed or clinical examination shows no trace of the tumor or any metastatic foci. Then TNF is administered, alone or in combination with an auxiliary agent such as alpha-, beta-, or gamma-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, such as anti-protein C antibody, anti-protein S antibody, or C4b binding protein (see, WO 91/01753, published 21 Feb. 1991), or heat or radiation.

The effective amounts of the therapeutic agents administered in combination with a ZPA polypeptide or antagonist thereof will be at the physician's or veterinarian's discretion. Dosage administration and adjustment is done to achieve maximal management of the conditions to be treated. The dose will additionally depend on such factors as the type of the therapeutic agent to be used and the specific patient being treated. Typically, the amount employed will be the same dose as that used, if the given therapeutic agent is administered without a ZPA polypeptide.

### Articles of Manufacture

An article of manufacture such as a kit containing one or more ZPA polypeptides or agonists or antagonists thereof useful for the diagnosis or treatment of the disorders described above comprises at least a container and a label. Suitable containers include, for example, bottles, vials, syringes, and test tubes. The containers can be formed from a variety of materials such as glass or plastic. The container holds a composition that is effective for diagnosing or treating the condition and can have a sterile access port (for example, the container can be an intravenous solution bag or a vial having a stopper pierceable by a hypodermic injection needle). The active agent in the composition is one or more ZPA polypeptides or an agonist or antagonist thereto. The label on, or associated with, the container indicates that the composition is used for diagnosing or treating the condition of choice. The article of manufacture can further comprise a second container comprising a pharmaceutically-acceptable buffer, such as phosphate-buffered saline, Ringer's solution, and dextrose solution. It can further include other materials desirable from a commercial and user standpoint, including other buffers, diluents, filters, needles, syringes, and package inserts with instructions for use. The article of manufacture can also comprise a second or third container with another active agent as described above.

### Polyclonal Antibodies

Methods of preparing polyclonal antibodies are known to the ordinarily skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent can include a ZPA polypeptide or a fusion protein thereof. It can be useful to conjugate the immunizing agent to a protein known to be immunogenic in the mammal being immunized. Examples of such immunogenic proteins include, but are not limited to, keyhole limpet hemocyanin, serum albumin, bovine thyroglobulin, and soybean trypsin inhibitor. Examples of adjuvants that can be employed include Freund's complete adjuvant and MPL-TDM adjuvant (monophosphoryl Lipid A or synthetic trehalose dicorynomycolate). The immunization protocol can be selected by one skilled in the art without undue experimentation.

### Monoclonal Antibodies

Anti-ZPA antibodies can be monoclonal antibodies. Monoclonal antibodies can be prepared, e.g., using hybridoma methods, such as those described by Kohler and Milstein, Nature, 256:495 (1975) or can be made by recombinant DNA methods (U.S. Pat. No. 4,816,567) or can be produced by the methods described herein in the Example section. In a hybridoma method, a mouse, hamster, or other appropriate host animal is typically immunized with an immunizing agent to elicit lymphocytes that produce or are capable of producing antibodies that will specifically bind to the immunizing agent. Alternatively, the lymphocytes can be immunized in vitro.

The immunizing agent will typically include a ZPA polypeptide or a fusion protein thereof. Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell. Goding, Monoclonal Antibodies: Principles and Practice (New York: Academic Press, 1986), pp. 59-103 Immortalized cell lines are usually transformed mammalian cells, particularly myeloma cells of rodent, bovine, and human origin. Usually, rat or mouse myeloma cell lines are employed. The hybridoma cells can be cultured in a suitable culture medium that contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Exemplary immortalized cell lines are those that fuse efficiently, support stable high-level expression of antibody by the selected antibody-producing cells, and are sensitive to a medium such as HAT medium. In certain embodiments, immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. Kozbor, J. Immunol., 133:3001 (1984); Brodeur et al., Monoclonal Antibody Production Techniques and Applications (Marcel Dekker, Inc.: New York, 1987) pp. 51-63.

The culture medium in which the hybridoma cells are cultured can then be assayed for the presence of monoclonal antibodies directed against a ZPA polypeptide. In certain embodiments, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980).

After the desired hybridoma cells are identified, the clones can be subcloned by limiting dilution procedures and grown by standard methods. Goding, supra. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention can serve as a source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy- and light-chain constant domains in place of the homologous murine sequences (U.S. Pat. No. 4,816,567; Morrison et al., supra) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

The antibodies can be monovalent antibodies. Methods for preparing monovalent antibodies are known in the art. For example, one method involves recombinant expression of immunoglobulin light chain and modified heavy chain. The heavy chain is truncated generally at any point in the Fc region so as to prevent heavy-chain crosslinking. Alternatively, the relevant cysteine residues are substituted with another amino acid residue or are deleted so as to prevent crosslinking.

In vitro methods are also suitable for preparing monovalent antibodies. Digestion of antibodies to produce fragments thereof, particularly Fab fragments, can be accomplished using techniques known in the art.

### Human and Humanized Antibodies

The anti-ZPA antibodies can further comprise humanized antibodies or human antibodies. Humanized forms of non-human (e.g., murine) antibodies are chimeric immunoglobulins, immunoglobulin chains, or fragments thereof (such as Fv, Fab, Fab′, F(ab′)₂, or other antigen-binding subsequences of antibodies) that contain minimal sequence derived from non-human immunoglobulin. Humanized antibodies include human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework residues of the human immunoglobulin are replaced by corresponding non-human residues. Humanized antibodies can also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin, and all or substantially all of the FR regions are those of a human immunoglobulin consensus sequence. The humanized antibody can also comprise at least a portion of an immunoglobulin constant region (Fc), typically that of a human immunoglobulin. Jones et al., Nature, 321: 522-525 (1986); Riechmann et al., Nature, 332: 323-329 (1988); Presta, Curr. Op. Struct. Biol., 2:593-596 (1992).

Methods for humanizing non-human antibodies are known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321: 522-525 (1986); Riechmann et al., Nature, 332: 323-327 (1988); Verhoeyen et al., Science, 239: 1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806, 5,569,825, 5,591,669 (all of GenPharm); 5,545,807; and WO 97/17852. Alternatively, human antibodies can be made by introducing human immunoglobulin loci into transgenic animals, e.g., mice in which the endogenous immunoglobulin genes have been partially or completely inactivated. Upon challenge, human antibody production is observed that closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; and 5,661,016, and in the following scientific publications: Marks et al., Bio/Technology, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); Lonberg and Huszar, Intern. Rev. Immunol., 13: 65-93 (1995).

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S, and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Human antibodies can also be produced using various techniques known in the art, including phage display libraries. Hoogenboom and Winter, J. Mol. Biol., 227: 381 (1991); Marks et al., J. Mol. Biol., 222: 581 (1991). The techniques of Cole et al. and Boerner et al. are also available for the preparation of human monoclonal antibodies. Cole et al., Monoclonal Antibodies and Cancer Therapy, Alan R. Liss, p. 77 (1985) and Boerner et al., J. Immunol., 147(1): 86-95 (1991).

Bispecific anti-ZPA Antibodies

Bispecific antibodies are monoclonal, optionally human or humanized, antibodies that have binding specificities for at least two different antigens. In the present case, one of the binding specificities is for a ZPA polypeptide, the other one is for any other antigen.

Methods for making bispecific antibodies are known in the art. Traditionally, the recombinant production of bispecific antibodies is based on the co-expression of two immunoglobulin heavy-chain/light-chain pairs, where the two heavy chains have different specificities. Milstein and Cuello, Nature, 305: 537-539 (1983). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10: 3655-3659 (1991).

Antibody variable domains with the desired binding specificities (antibody-antigen combining sites) can be fused to immunoglobulin constant-domain sequences. In certain embodiments, the fusion is with an immunoglobulin heavy-chain constant domain, comprising at least part of the hinge, CH2, and CH3 regions. In certain embodiments, the first heavy-chain constant region (CH1) containing the site necessary for light-chain binding is present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies, see, for example, Suresh et al., Methods in Enzymology, 121: 210 (1986).

Various techniques for making and isolating bispecific antibody fragments directly from recombinant cell culture have also been described. For example, bispecific antibodies have been produced using leucine zippers. Kostelny et al., J. Immunol., 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a VH connected to a VL by a linker which is too short to allow pairing between the two domains on the same chain. Accordingly, the VH and VL domains of one fragment are forced to pair with the complementary VL and VH domains of another fragment, thereby forming two antigen-binding sites. Another strategy for making bispecific antibody fragments by the use of single-chain Fv (sFv) dimers has also been reported. Sec Gruber et al., J. Immunol., 152:5368 (1994).

Antibodies with more than two valencies are contemplated. For example, trispecific antibodies can be prepared. Tutt et al. J. Immunol. 147: 60 (1991)

### Heteroconjugate Antibodies

Heteroconjugate antibodies are composed of two covalently joined antibodies. Such antibodies have, for example, been proposed to target immune-system cells to unwanted cells (U.S. Pat. No. 4,676,980), and for treatment of HIV infection. WO 91/00360; WO 92/200373; EP 03089. It is contemplated that the antibodies can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

### Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See, Caron et al., J. Exp. Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al., Cancer Research, 53: 2560-2565 (1993). Alternatively, an antibody can be engineered that has dual Fc regions and can thereby have enhanced complement lysis and ADCC capabilities. See, Stevenson et al., Anti-Cancer Drug Design, 3: 219-230 (1989).

### Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody can be conjugated to a “receptor” (such as streptavidin) for utilization in tumor pretargeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

### Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See, Gabizon et al., J. National Cancer Inst., 81(19): 1484 (1989).

### Pharmaceutical Compositions of Antibodies

Antibodies specifically binding a ZPA polypeptide identified herein, as well as other molecules identified by the screening assays disclosed hereinbefore, can be administered for the treatment of various disorders as noted above and below in the form of pharmaceutical compositions.

Lipofectins or liposomes can be used to deliver the polypeptides, nucleic acid molecules, antibodies, antagonists or composition of this invention into cells. Where antibody fragments are used, the smallest inhibitory fragment that specifically binds to the binding domain of the target protein can be used. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences, supra.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they can denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization can be achieved by modifying sulfhydryl residues, lyophilizing from acidic solutions, controlling moisture content, using appropriate additives, and developing specific polymer matrix compositions.

### Methods of Treatment Using an Anti-ZPA Antibody or Fragment Thereof

It is contemplated that the antibodies to a ZPA polypeptide can be used to treat various apoptosis-related disorders as noted above. It will be appreciated that antigen-binding fragments of an anti-ZPA polypeptide antibody can also be used in the following methods.

The antibodies are administered to a mammal, e.g. a human, in accord with known methods, such as intravenous administration as a bolus or by continuous infusion over a period of time, by intramuscular, intraperitoneal, intracerobrospinal, intravenous, subcutaneous, intra-articular, intrasynovial, intrathecal, oral, topical, or inhalation routes.

The location of the binding target of an antibody of the invention may be taken into consideration in preparation and administration of the antibody. When the binding target is an intracellular molecule, certain embodiments of the invention provide for the antibody or antigen-binding fragment thereof to be introduced into the cell where the binding target is located. In one embodiment, an antibody of the invention can be expressed intracellularly as an intrabody. The term “intrabody,” as used herein, refers to an antibody or antigen-binding portion thereof that is expressed intracellularly and that is capable of selectively binding to a target molecule, as described in Marasco, Gene Therapy 4: 11-15 (1997); Kontermann, Methods 34: 163-170 (2004); U.S. Pat. Nos. 6,004,940 and 6,329,173; U.S. Patent Application Publication No. 2003/0104402, and PCT Publication No. WO2003/077945. Intracellular expression of an intrabody is effected by introducing a nucleic acid encoding the desired antibody or antigen-binding portion thereof (lacking the wild-type leader sequence and secretory signals normally associated with the gene encoding that antibody or antigen-binding fragment) into a target cell. Any standard method of introducing nucleic acids into a cell may be used, including, but not limited to, microinjection, ballistic injection, electroporation, calcium phosphate precipitation, liposomes, and transfection with retroviral, adenoviral, adeno-associated viral and vaccinia vectors carrying the nucleic acid of interest. One or more nucleic acids encoding all or a portion of an anti-ZPA antibody of the invention can be delivered to a target cell, such that one or more intrabodies are expressed which are capable of intracellular binding to a ZPA protein and modulation of one or more ZPA-mediated cellular pathways.

In another embodiment, internalizing antibodies are provided. Antibodies can possess certain characteristics that enhance delivery of antibodies into cells, or can be modified to possess such characteristics. Techniques for achieving this are known in the art. For example, cationization of an antibody is known to facilitate its uptake into cells (see, e.g., U.S. Pat. No. 6,703,019). Lipofections or liposomes can also be used to deliver the antibody into cells. Where antibody fragments are used, the smallest inhibitory fragment that specifically binds to the binding domain of the target protein is generally advantageous. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993).

Entry of antibodies into target cells can be enhanced by methods known in the art. For example, certain sequences, such as those derived from HIV Tat or the Antennapedia homeodomain protein are able to direct efficient uptake of heterologous proteins across cell membranes. See, e.g., Chen et al., Proc. Natl. Acad. Sci. USA (1999), 96:4325-4329.

When the binding target is located in the brain, certain embodiments of the invention provide for the antibody or antigen-binding fragment thereof to traverse the blood-brain barrier. Certain neurodegenerative diseases are associated with an increase in permeability of the blood-brain barrier, such that the antibody or antigen-binding fragment can be readily introduced to the brain. When the blood-brain barrier remains intact, several art-known approaches exist for transporting molecules across it, including, but not limited to, physical methods, lipid-based methods, and receptor and channel-based methods.

Physical methods of transporting the antibody or antigen-binding fragment across the blood-brain barrier include, but are not limited to, circumventing the blood-brain barrier entirely, or by creating openings in the blood-brain barrier. Circumvention methods include, but are not limited to, direct injection into the brain (see, e.g., Papanastassiou et al., Gene Therapy 9: 398-406 (2002)), interstitial infusion/convection-enhanced delivery (see, e.g., Bobo et al., Proc. Natl. Acad. Sci. USA 91: 2076-2080 (1994)), and implanting a delivery device in the brain (see, e.g., Gill et al., Nature Med. 9: 589-595 (2003); and Gliadel Wafers™, Guildford Pharmaceutical). Methods of creating openings in the barrier include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A-7 (see, e.g., U.S. Pat. Nos. 5,112,596, 5,268,164, 5,506,206, and 5,686,416), and transfection of neurons that straddle the blood-brain barrier with vectors containing genes encoding the antibody or antigen-binding fragment (see, e.g., U.S. Patent Publication No. 2003/0083299).

Lipid-based methods of transporting the antibody or antigen-binding fragment across the blood-brain barrier include, but are not limited to, encapsulating the antibody or antigen-binding fragment in liposomes that are coupled to antibody binding fragments that bind to receptors on the vascular endothelium of the blood-brain barrier (see, e.g., U.S. Patent Application Publication No. 20020025313), and coating the antibody or antigen-binding fragment in low-density lipoprotein particles (see, e.g., U.S. Patent Application Publication No. 20040204354) or apolipoprotein E (see, e.g., U.S. Patent Application Publication No. 20040131692).

Receptor and channel-based methods of transporting the antibody or antigen-binding fragment across the blood-brain barrier include, but are not limited to, using glucocorticoid blockers to increase permeability of the blood-brain barrier (see, e.g., U.S. Patent Application Publication Nos. 2002/0065259, 2003/0162695, and 2005/0124533); activating potassium channels (see, e.g., U.S. Patent Application Publication No. 2005/0089473), inhibiting ABC drug transporters (see, e.g., U.S. Patent Application Publication No. 2003/0073713); coating antibodies with a transferrin and modulating activity of the one or more transferrin receptors (see, e.g., U.S. Patent Application Publication No. 2003/0129186), and cationizing the antibodies (see, e.g., U.S. Pat. No. 5,004,697).

Other therapeutic regimens can be combined with the administration of the antibodies of the instant invention as noted above. For example, if the antibodies are to treat cancer, the patient to be treated with such antibodies can also receive radiation therapy. Alternatively, or in addition, a chemotherapeutic agent can be administered to the patient. Preparation and dosing schedules for such chemotherapeutic agents can be used according to manufacturers' instructions or as determined empirically by the skilled practitioner. Preparation and dosing schedules for such chemotherapy are also described in Chemotherapy Service, Ed., M. C. Perry (Williams & Wilkins: Baltimore, Md., 1992). The chemotherapeutic agent can precede, or follow administration of the antibody, or can be given simultaneously therewith. The antibody can be combined with an anti-estrogen compound such as tamoxifen or EVISTA™ or an anti-progesterone such as onapristone (see, EP 616812) in dosages known for such molecules.

If the antibodies are used for treating cancer, they can be, optionally, administered with antibodies against one or more tumor-associated antigens, such as antibodies that bind to one or more of the ErbB2, EGFR, ErbB3, ErbB4, or VEGF receptor(s). These also include the agents set forth above. Also, the antibody is suitably administered serially or in combination with radiological treatments, whether involving irradiation or administration of radioactive substances. Alternatively, or in addition, two or more antibodies binding the same or two or more different antigens disclosed herein can be co-administered to the patient. In one embodiment, the antibodies herein are co-administered with a growth-inhibitory agent. For example, the growth-inhibitory agent can be administered first, followed by an antibody of the present invention. However, simultaneous administration or administration of the antibody of the present invention first is also contemplated. Suitable dosages for the growth-inhibitory agent are those presently used and can be lowered due to the combined action (synergy) of the growth-inhibitory agent and the antibody herein.

In one embodiment, vascularization of tumors is attacked in combination therapy. An anti-ZPA polypeptide antibody and another antibody (e.g., anti-VEGF) are administered to tumor-bearing patients at therapeutically effective doses as determined, for example, by observing necrosis of the tumor or its metastatic foci, if any. This therapy is continued until such time as no further beneficial effect is observed or clinical examination shows no trace of the tumor or any metastatic foci. Then TNF is administered, alone or in combination with an auxiliary agent such as alpha-, beta-, or gamma-interferon, anti-HER2 antibody, heregulin, anti-heregulin antibody, D-factor, interleukin-1 (IL-1), interleukin-2 (IL-2), granulocyte-macrophage colony stimulating factor (GM-CSF), or agents that promote microvascular coagulation in tumors, (such as anti-protein C antibody, anti-protein S antibody, or C4b binding protein, see, WO 91/01753, published 21 Feb. 1991), or heat or radiation.

Since the auxiliary agents will vary in their effectiveness, it can be desirable to compare their impact on the tumor by matrix screening in conventional fashion. The administration of an anti-ZPA polypeptide antibody and TNF is repeated until the desired clinical effect is achieved. Alternatively, an anti-ZPA polypeptide antibody is administered together with TNF and, optionally, auxiliary agent(s). In instances where solid tumors are found in the limbs or in other locations susceptible to isolation from the general circulation, the therapeutic agents described herein are administered to the isolated tumor or organ. In other embodiments, a FGF or PDGF antagonist, such as an anti-FGF or an anti-PDGF neutralizing antibody, is administered to the patient in conjunction with an anti-ZPA polypeptide antibody.

For the prevention or treatment of an apoptosis-related disorder, the appropriate dosage of an antibody herein will depend on the type of disorder to be treated, as defined above, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

For example, depending on the type and severity of the disorder, about 1 μg/kg to 50 mg/kg (e.g., 0.1-20 mg/kg) of antibody is an initial candidate dosage for administration to the patient, whether, for example, by one or more separate administrations, or by continuous infusion. A typical daily or weekly dosage might range from about 1 μg/kg to 100 mg/kg or more, depending on the factors mentioned above. For repeated administrations over several days or longer, depending on the condition, the treatment is repeated or sustained until a desired suppression of disorder symptoms occurs. However, other dosage regimens can be useful. The progress of this therapy is easily monitored by conventional techniques and assays, including, for example, radiographic tumor imaging.

Articles of Manufacture with Antibodies

An article of manufacture comprising a container with the antibody and a label is also provided. Such articles are described above, wherein the active agent is an anti-ZPA antibody.

### Diagnosis and Prognosis of Apoptosis-Related Disorders Using Antibodies

Antibodies directed against one or more ZPA polypeptides can be used to diagnose and/or determine the prognosis of an apoptosis-related disorder. For example, antibodies directed against one or more ZPA polypeptides can be used as tumor diagnostics or prognostics.

For example, antibodies, including antigen-binding antibody fragments, can be used qualitatively or quantitatively to detect the expression of genes including the gene encoding the ZPA polypeptide, either in intact cells or in cell lysates. In certain embodiments, the antibody is equipped with a detectable label, e.g., a fluorescent label, and binding can be monitored by microscopy, flow cytometry, fluorimetry, or other techniques known in the art. Such binding assays are performed essentially as described above.

All publications (including patents and patent applications) cited herein are hereby incorporated in their entirety by reference.

Commercially available reagents referred to in the Examples were used according to manufacturer's instructions unless otherwise indicated. The source of those cells identified in the following Examples, and throughout the specification, by ATCC accession numbers is the American Type Culture Collection, Manassas, Va. Unless otherwise noted, the present invention uses standard procedures of recombinant DNA technology, such as those described hereinabove and in the following textbooks: Sambrook et al., supra; Ausubel et al., Current Protocols in Molecular Biology (Green Publishing Associates and Wiley Interscience, N.Y., 1989); Innis et al., PCR Protocols: A Guide to Methods and Applications (Academic Press, Inc.: N.Y., 1990); Harlow et al., Antibodies: A Laboratory Manual (Cold Spring Harbor Press: Cold Spring Harbor, 1988); Gait, Oligonucleotide Synthesis (IRL Press: Oxford, 1984); Freshney, Animal Cell Culture, 1987; Coligan et al., Current Protocols in Immunology, 1991.

Throughout this specification and claims, the word “comprise,” or variations such as “comprises” or “comprising,” will be understood to imply the inclusion of a stated integer or group of integers but not the exclusion of any other integer or group of integers.

The foregoing written description is considered to be sufficient to enable one skilled in the art to practice the invention. The following Examples are offered for illustrative purposes only, and are not intended to limit the scope of the present invention in any way. Indeed, various modifications of the invention in addition to those shown and described herein will become apparent to those skilled in the art from the foregoing description and fall within the scope of the appended claims.

### EXAMPLES

### Example 1

### Identification of Putative Zebrafish Bcl-2 Family Members

Homologs of certain members of the human Bcl-2 family had previously been identified in zebrafish using traditional sequence searching methodologies (Inohara and Nunez, Cell Death Diff. 7: 509-510 (2000); Coultas et al., Cell Death Diff. 9: 1163-1166 (2002); Aouacheria et al., Mol. Biol. Evol. 22(12): 2395-2416 (2005)). Despite significant effort by several groups, equivalents to many proteins important in the mammalian intrinsic apoptotic pathway had not been found in zebrafish (id.). Applicants suspected the existence of novel and divergent Bcl-2 genes in zebrafish that would not appear in the most common sequence databases and that would not be readily discoverable by traditional sequence-based searching. A comprehensive search for these sequences using both traditional BLAST and PROSITE searching in a customized sequence database and feature-based database mining with customized Hidden Markov Models in conjunction with the Unison database (http://unison-db.org) was therefore undertaken.

a. BLAST/PROSITE Database Mining

Previous BLAST and PROSITE searching by other groups had failed to identify zebrafish homologs for several Bcl-2 family members, including Bak, Bik, Bim, and PUMA (Inohara and Nunez, Cell Death Diff. 7: 509-510 (2000); Coultas et al., Cell Death Diff. 9: 1163-1166 (2002); Aouacheria et al., Mol. Biol. Evol. 22(12): 2395-2416 (2005)). To investigate whether the database used for those prior studies had been insufficiently broad, a custom database containing 136,655 zebrafish amino acid sequences was constructed. The custom database was queried by standard BLAST (version 2.2.10) and PROSITE (release 18) searching techniques using default arguments and known human, mouse, and chicken members of the Bcl-2 family as queries.

This search identified many sequences, two of which had not previously been identified in similar searches for Bcl-2-related sequences in zebrafish. The first sequence (Ensembl:ENSDARP00000040899 (SEQ ID NO: 1)) was 33% identical to human Bax over 51% of the sequence with an e-value of 1e-14. None of the PROSITE BH patterns aligned to ENSDARP00000040899, and the Ensembl database (available at www.ensembl.org; Birney et al., Nucleic Acids Res. 34: D556-561 (2006)) annotates that gene as hypothetical. The second sequence (XP_{—}693331 (SEQ ID NO: 3)) was 33% identical to human Bad over 68% of the sequence with an e-value of 1.3.

Searching with PROSITE patterns did not identify any of the known zebrafish Bcl-2 family members and no new zebrafish sequences were identified using PROSITE patterns alone.

b. Feature-Based Database Mining

Even using a customized sequence database tailored to zebrafish sequences (described in Example 1(a)), only a single new putative Bcl-2 family member was identified. Because standard BLAST and PROSITE database searching were apparently unable to identify further zebrafish Bcl-2 members, the possibility that family members may have significantly different overall base sequences in zebrafish and humans, yet still share function was considered. To address this possibility, feature-based sequence mining was employed. Feature-based mining identifies sequences that match a set of specified features.

(1) Feature-Based Mining Sequence Sources

Feature-based mining was performed in the Unison database. Briefly, Unison comprises a non-redundant compendium of sequences from many source databases and extensive precomputed proteomic predictions within a relational database (http://unison-db.org). The Unison schema, non-proprietary data and predictions, tools, and Internet interface have been released under the Academic Free License and are available for use or download at http://unison-db.org/. At the time of the study, Unison included 6.5 million distinct sequences, including 136,655 distinct zebrafish sequences from the RefSeq database (Wheeler et al., Nucl. Acid Res. 34: D173-180 (2006)), the UniProt/Swiss-Pro and UniProt/TrEMBL databases (Wu, Nucl. Acids Res. 34: D187-191 (2006)), and Ensembl Release 35 (Birney et al., Nucleic Acids Res. 34: D556-561 (2006)).

(2) Construction of Custom Hidden Markov Models for Bcl-2 and BH Domains

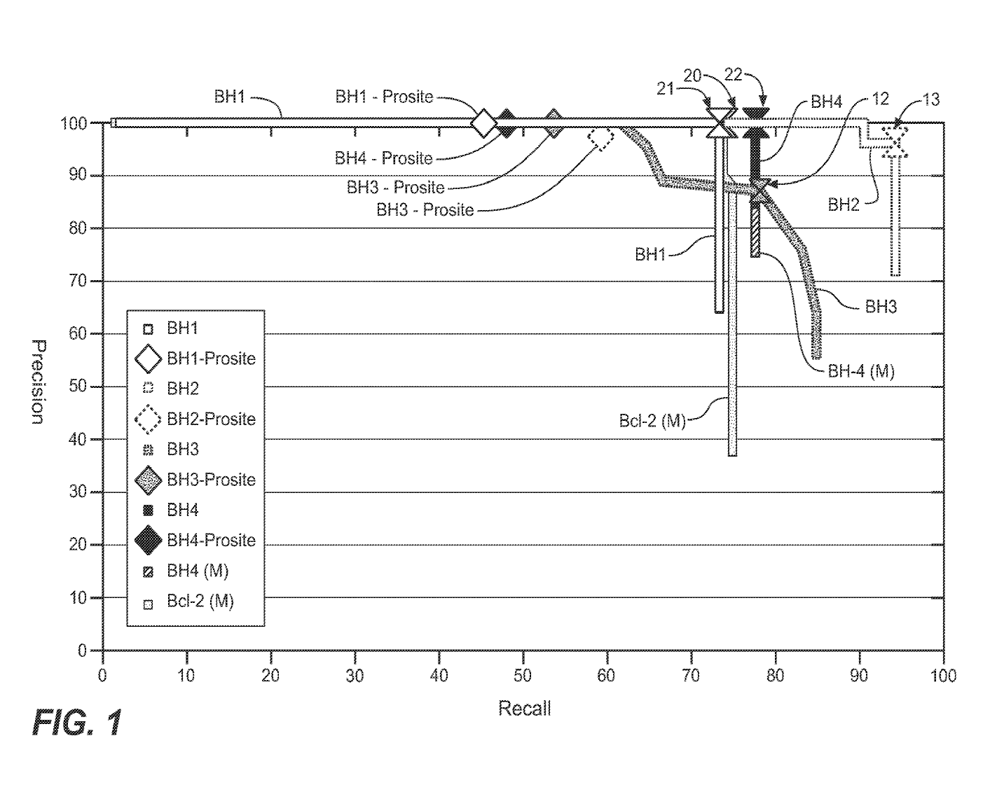

Multiple sequence alignments were constructed from PROSITE patterns and matrices (Sigrist et al., Brief Bioinform. 3: 265-274 (2002)). For patterns PS01080 (BH1), PS01258 (BH2), PS01259 (BH3) and PS01260 (BH4), and for the matrix PS50063 (BH4), the false negative sequences were manually incorporated into the alignment of true positive sequences obtained from the PROSITE website (http://us.expasy.org/cgi-bin/nicedoc.po?pdoc00829). For the PS50062 (Bcl-2) matrix, manual alignment of the three false negatives was not obvious and only the true positives were used. The alignments were used to build “global” hidden Markov models (HMM)s using the hmmbuild and hmmcalibrate programs from HMMER v.23.2 (Eddy, Bioinformatics 14:755-763 (1998)) incorporating the default arguments. In order to assess recall and precision, and to determine appropriate score thresholds, the six constructed HMMs were aligned to the UniProt/Swiss-Prot sequence database (FIG. 1). The results demonstrated that the constructed HMMs had improved recall without loss of precision at suitable score thresholds. From the results HMM alignment score cutoffs were selected as follows: BH1: 21; BH2: 13; BH3: 12; BH4: 22; BH4 matrix: 20; and Bcl2 matrix: 20.

Unison's cluster-based update framework was used to run hmmsearch results for all 136,655 zebrafish sequences. HMM alignments for all zebrafish sequences were computed and loaded into an in-house copy of Unison using Unison's automated update facility. A transmembrane domain analysis was also performed using TMHMM v. 2.0c (Krogh et al., J. Mol. Biol. 305: 567-580 (2001)) with the default options. Once the results were loaded, feature-based mining involved framing SQL queries to represent appropriate conjunctions of precomputed proteomic predictions. A query was created to identify all zebrafish sequences which aligned to any of the HMMs using the score criteria defined above. The query identified each sequence that overlapped genomically with a known Bcl-2 family member. The four most promising candidate genes not previously identified as zebrafish Bcl-2 family members were analyzed further.

### Example 2

### Analysis of Identified Sequences

Each of the putative Bcl-2 related (“B2R”) zebrafish genes (ENSDARP0000040899 and XP_{—}693331 identified from the BLAST searching described in Example 1(a) and ENSDARP00000066976, FGENESH00000065416, FGENESH00000065416, and FGENESH00000082230 identified from the feature-based mining described in Example 1(b)) were further analyzed to assign a specific identity as a particular member of the Bcl-2 family. Each sequence was subjected to BLAST searching, and aligned with the most homologous identified sequence, resulting in an e-value, a score, a percent identity, and a percent coverage (see Table 2). The presence or absence of a putative transmembrane domain within the encoded protein was determined. The neighboring genes within the zebrafish genome were also examined so that any conserved synteny could be assessed (see Table 2).

a. zBak (ENSDARP00000040899)

BLAST searching (described in Example 1(a)) identified the sequence ENSDARP00000040899 as being 33% identical to human Bax over 51% of the sequence with an e-value of 1e-14 (see Table 2). The amino acid sequence of ENSDARP00000040899 and its encoding nucleotide sequence are shown below.

ENSDARP00000040899:
(SEQ ID NO: 1)
MACEASQDDQIGEALLIGVVRQELMEVMEVTEGNAAPPALPEAKPISNSQ
DQILVQQLANTIKVIGDKLDQDQAFNDMIDGLVKVADKSSFWKLVEKVFT
DGQINWGRIIVLFYSVGKLSAKMVVARLPRIVSDILSLSLDYFKRNLLQW
IRTVGGWMNSIPALACFSVDQFSGSSMRKYSPYVGVVFAFTGGLLLGGFI
VSRFQKT
ENSDARG00000030881:
(SEQ ID NO: 2)
ATGGCTTGTGAAGCCTCACAGGATGATCAGATTGGAGAGGCACTCTTAAT
AGGGGTAGTAAGGCAGGAGCTAATGGAGGTGATGGAGGTGACTGAAGGAA
ATGCAGCTCCTCCAGCTCTTCCTGAAGCTAAACCAATAAGCAACAGCCAG
GACCAGATTCTGGTTCAGCAGCTGGCGAACACAATCAAAGTGATCGGTGA
CAAACTCGACCAGGATCAAGCATTTAACGACATGATCGATGGCTTAGTAA
AGGTAGCTGATAAAAGCAGTTTCTGGAAACTTGTGGAAAAGGTGTTCACA
GATGGCCAGATCAACTGGGGCAGAATTATCGTGCTGTTTTATTCTGTTGG
AAAACTGTCAGCCAAGATGGTCGTCGCTCGCCTACCCAGAATTGTTTCAG
ATATTTTATCATTAAGTCTTGATTACTTCAAAAGGAATCTGTTGCAGTGG
ATTCGCACAGTAGGAGGATGGATGAACAGTATCCCTGCACTGGCCTGTTT
CTCTGTTGACCAATTTTCTGGTTCTTCAATGAGAAAATATTCTCCTTACG
TTGGAGTTGTGTTTGCCTTCACTGGTGGCCTACTGCTGGGTGGCTTCATC
GTCTCGAGATTTCAGAAAACCTGA

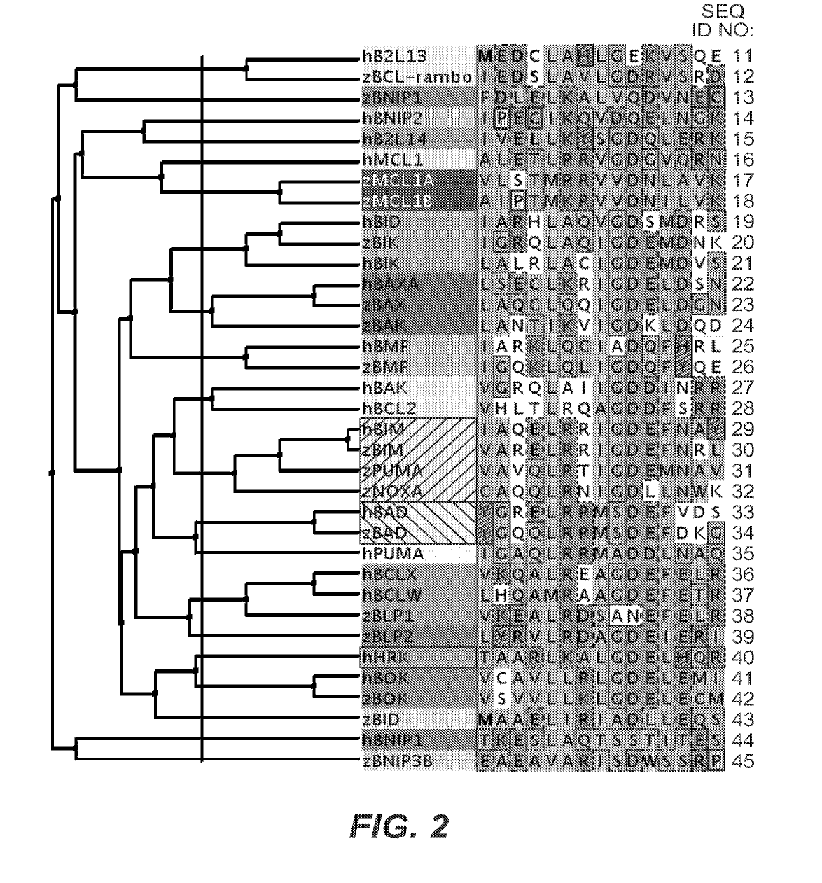

Ensembl annotated ENSDARP00000040899 as hypothetical, with no ascribed identity. None of the PROSITE BH patterns aligned to the sequence. Of the HMMs constructed in Example 1(b), the Bcl-2, BH1, BH2, and BH3 HMMs each aligned to the sequence with scores of 33, 14, 6.1, and 2.4, respectively, and e-values of 7.5e-7, 0.031, 110, and 1300, respectively. An alignment of the sequence was also made with the BH3 domains of several known or proposed Bcl-2 family members, and examined using Jalview 2.05 (Clamp et al., Bioinformatics 12: 426-427 (2004)) (FIG. 2). Despite the poor score of the BH3 HMM alignment to ENSDAR00000040899, the sequence showed qualitatively good similarity with the BH3 domains of other Bcl-2 family members and plausible amino acid substitutions at all positions which were not conserved (see FIG. 2). TMHMM analysis also predicted that the sequence contained a transmembrane domain from about amino acids 180-202.

The presence of a conserved syntenic relationship was examined for ENSDAR00000040899 and all other sequences described herein by comparing flanking genes for the zebrafish gene to flanking genes for the human functional homologue using the publicly available ENSEMBL database (available at www.ensembl.org; Birney et al., Nucleic Acids Res. 34: D556-561 (2006)). The zebrafish gene was designated to share a conserved syntenic relationship with the human gene if more than a single flanking gene on either or both sides of the zebrafish gene had a human equivalent that also flanked the human gene. ENSDAR00000040899 was not in a conserved syntenic relationship with human Bak, but was to human Bax.

ENSDAR00000040899 was also included in an alignment of candidate zebrafish Bcl-2 family members against human Bcl-2 sequences, and vice versa, using BLAST to identify reciprocal best BLAST alignments, pairwise identity, and alignment coverage (Table 2). Although alignment of the candidate sequence to human Bax resulted in the best e-value, the best overall sequence coverage was obtained by an alignment to human Bak. Although the BH2 and BH3 HMM alignments did not score significantly, the presence of those domains in the context of significant scores for Bcl-2 and BH1, the absence of a predicted BH4 domain, the similarity of the BH3 domain with those of other Bcl-2 members, the prediction of a TM domain, and the reciprocal BLAST analysis combined to suggest that ENSDAR00000040899 was a pro-apoptotic member of the Bcl-2 family, most likely functionally homologous to Bak.

b. zBad2 (XP_{—}693331)

As described in Example 1(a), XP_{—}693331 was also identified by a BLAST search in a customized zebrafish sequence database. The amino acid sequence of XP_{—}693331 and the mRNA sequence encoding it are set forth below.

XP_693331:
(SEQ ID NO: 3)
MENTSHDHQDDSSTLDEKERSHLKGTIKNHGQHQDRTSANISPQGRVRLY
SESQVYTVSRWQDTETQDGASVEENGDGLPFRGRSQSAPAALWKAKKYGR
QLRRMSDEFDTWLDKGEVKRANSQKQTYRGWFSFLWSPKEEEGRE
XM_688239 (nucleotides 367-804):
(SEQ ID NO: 4)
ATGGAGAACACCTCGCATGACCATCAAGATGATTCCAGCACCTTGGATGA
AAAAGAGAGATCACATCTGAAAGGGACAATCAAGAACCATGGACAACATC
AGGATCGAACATCGGCCAACATTTCTCCTCAAGGGCGTGTGCGGCTCTAT
TCGGAATCTCAAGTGTATACAGTCAGCCGCTGGCAGGACACAGAGACCCA
GGATGGAGCATCGGTGGAGGAGAACGGAGATGGACTTCCATTCAGGGGTC
GTTCTCAATCAGCACCTGCTGCACTGTGGAAAGCAAAAAAGTATGGCCGT
CAGTTGAGGAGAATGAGCGATGAATTCGACACATGGCTCGATAAAGGGGA
GGTCAAGAGAGCGAACAGCCAGAAACAGACCTACCGAGGATGGTTTTCGT
TCCTCTGGAGTCCCAAAGAAGAAGAGGGCAGAGAATGA

The GenBank annotation for XP_{—}693331 predicted that the sequence was similar to the BH3-only proapoptotic protein, Bad. An alignment of XP_{—}693331 with human Bad showed that the two proteins have 33% identity over 72% of the sequence. The HMMer score versus the BH3 domain was 12.6, with an e-value of 1.3. A TMHMM analysis of XP_{—}693331 showed no predicted transmembrane domains in the sequence, similar to human Bad. A clone of XP_{—}693331 was obtained (Open Biosystems), and the surrounding sequences within the zebrafish genome were identified.

The genes adjacent to XP_{—}693331 differed from those adjacent to human Bad, thus XP_{—}693331 was not in a conserved syntenic relationship with human Bad. However, another zebrafish gene (A1332008) had previously been identified as having significant homology to human Bad and was syntenic to human bad (Inohara and Nunez, Cell Death Diff. 7: 509-510 (2000); Coultas et al., Cell Death Diff. 9: 1163-1166 (2002)). XP_{—}693331 was approximately 32% identical to that previously identified sequence. Thus, the above data suggested that XP_{—}693331 was a Bcl-2 family member related to human Bad. Because a zebrafish gene with sequence similarity to and conserved synteny to human Bad was previously known, XP 693331 was named “zBad2” as the second zebrafish gene with identity to human Bad.

c. zBik (ENSDARP00000066976)

The zebrafish sequence ENSDARP00000066976 was identified by feature-based database mining as described in Example 1(b) as a B2R protein. The amino acid sequence of ENSDARP00000066976 and the nucleotide sequence encoding it are set forth below.

ENSDARP00000066976:
(SEQ ID NO: 5)
MVEETRQQKNATTLQAGPAEVDHSNLYAFNMRVTQTIGRQLAQIGDEMDN
KWRQEPPVPWQNLNFGIYPYVLSRRVFSGRILANLWGSKIMPIFRTSWLL
PQLQNGCQEARKWAAWVSNLHVSDWSRSTTYTLASALLLVTVSIFLVNWN
EYEG
ENSDARG00000045549:
(SEQ ID NO: 6)
ATGGTGGAAGAAACTAGACAGCAGAAAAACGCCACAACCCTGCAGGCTGG
ACCTGCTGAGGTTGACCACAGTAATCTCTATGCATTCAATATGAGAGTCA
CCCAGACTATCGGACGACAGCTGGCTCAAATAGGGGACGAAATGGACAAT
AAATGGCGCCAAGAACCGCCTGTCCCATGGCAGAACCTGAATTTCGGGAT
TTATCCTTATGTCCTAAGTAGGAGAGTGTTCTCTGGAAGAATCCTCGCTA
ATCTTTGGGGGTCTAAGATTATGCCGATATTCAGGACGTCCTGGTTGCTT
CCACAGCTTCAAAATGGCTGTCAGGAGGCTAGAAAGTGGGCAGCTTGGGT
GTCCAACTTGCATGTTTCTGACTGGTCTCGCAGCACTACATACACCCTGG
CATCTGCTTTACTACTGGTCACTGTGTCTATCTTCCTTGTAAACTGGAAT
GAGTATGAAGGCTGA

BLAST searching in the customized zebrafish sequence database (as described in Example 1(a)) did not identify ENSDARP00000066976. TMHMM analysis of the sequence predicted the presence of a transmembrane domain from about amino acids 130-149. ENSDARP00000066976 did not match to the BH1, BH2, and BH4 HMMs. The sequence did match to the BH3 HMM with score of 18 and an e-value of 0.023. The presence of a BH3-related region and a transmembrane domain and an absence of BH1, BH2, and BH4-related regions suggested that ENSDARP00000066976 might be a member of the pro-apoptotic BH3-only subfamily of Bcl-2 proteins.

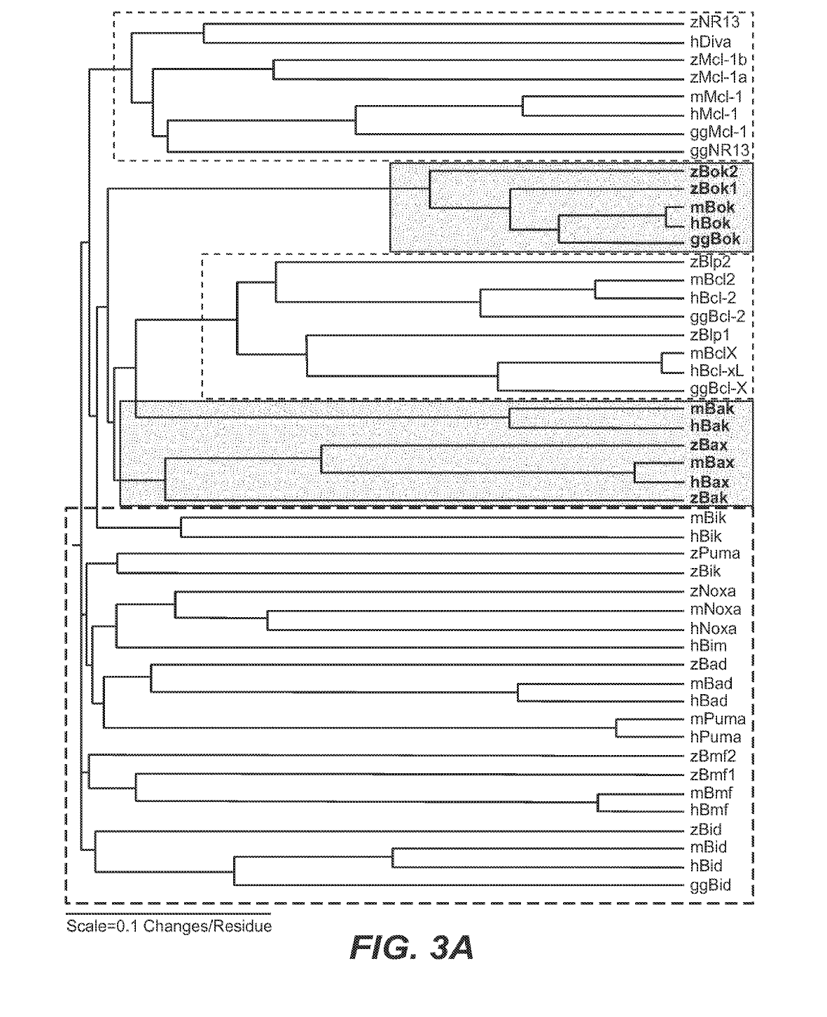

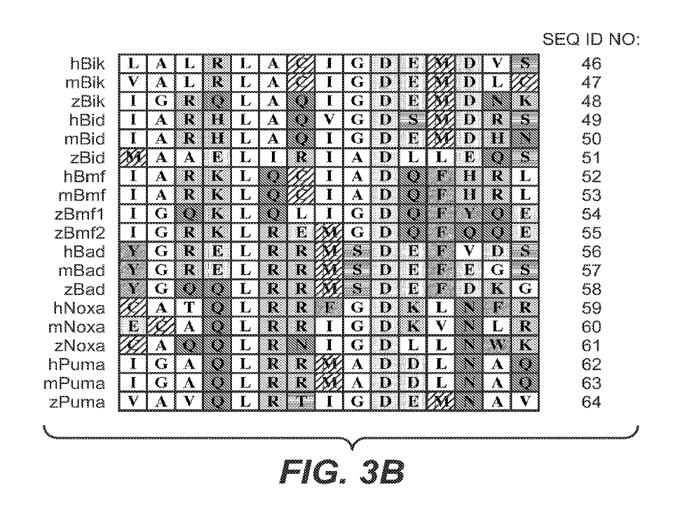

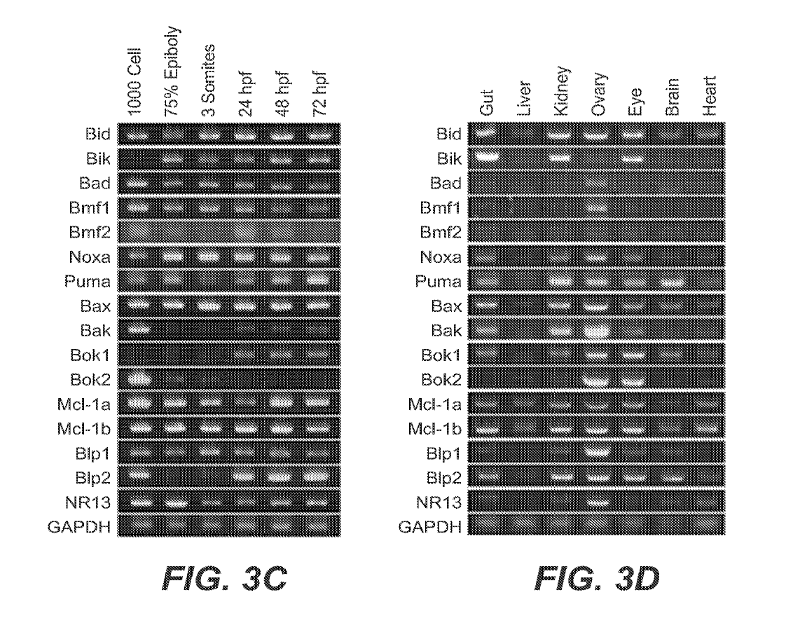

The ENSDARP00000066976 sequence in the zebrafish genome shared a conserved syntenic relationship with human Bik. The ENSDARP00000066976 putative BH3 region was compared with previously known human zebrafish Bcl-2 genes (see FIG. 2). The BH3 domain of ENSDARP00000066976 was most similar to that of human Bid despite the synteny and BLAST similarity with human Bik (FIG. 3B). However, based on a comparison of the two sequences, ENSDARP00000066976 lacked a putative caspasc cleavage site, while human Bid contains such a site. The presence of the BH3 domain, the absence of other BH domains, the transmembrane domain prediction, overall sequence similarity, and syntenic relationship analyses all suggested that ENSDARP00000066976 was a zebrafish pro-apoptotic BH3-only Bcl-2 subfamily member orthologous to human Bik.

d. zBim (FGENESH00000065416)

The zebrafish sequence FGENESH00000065416 was identified by feature-based database mining as described in Example 1(b) as a B2R protein. BLAST searching in the customized zebrafish sequence database (as described in Example 1(a)) did not identify FGENESH00000065416. FGENESH00000065416 did not match to the BH1, BH2, and BH4 HMMs. The sequence did match to the BH3 HMM with score of 18 and an e-value of 0.023. The presence of a BH3-related region and a transmembrane domain and an absence of BH1, BH2, and BH4-related regions suggested that FGENESH00000065416 might be a member of the pro-apoptotic BH3-only subfamily of Bcl-2 proteins.

BLAST alignments with the FGENESH00000065416 sequence show that the N-terminal half of the sequence was 32% identical to human Bim over 47% of human Bim with an e-value of 9e-7, further supporting the assignment of FGENESH00000065416 as a member of the pro-apoptotic BH3-only subfamily of Bcl-2 proteins. However, the C-terminal half of FGENESH00000065416 was most similar (32% identical) to developmentally-regulated RNA-binding protein 1, a mouse and rat RNA binding protein with putative involvement in neural development.

The juxtaposition of Bim homology and homology to an unrelated protein/function in the same contiguous region may be attributable to an assembly error within the zebrafish genome. In fact, no RNA product could be amplified from either the entire sequence or from the Bim-like fragment alone. Despite the apparent assembly error, the presence of the BH3 domain, the absence of other BH domains, and sequence similarity suggested that at least the N-terminal portion of FGENESH00000065416 was homologous to human Bim.

e. zPuma (FGENESH00000078270)

The zebrafish sequence FGENESH00000078270 was identified by feature-based database mining as described in Example 1(b) as a B2R protein. The amino acid sequence of FGENESH00000078270 and the nucleotide sequence encoding it (accession number CN323956) are set forth below.

FGENESH00000078270:
(SEQ ID NO: 7)
MTLCFLNTSAALADEEGDPLPTALINSLDLAVNQPVSGSGFCKLKLANEQ
TVVTLQQLATREPMGDEEEVQGFQSTDPHGTTVCGMARPEMESRVDEHNS
GTPNSCRMEVLRQDAWPNGSIIQPCHRRRTIATQTSTLSAPLPHIPSHDA
FSLDSVQQQDSLLRDNSGTEQEVSRPLPLPDLLADNQSSSEESTSSSSST
AEEDPTLEEQAVERVAVQLRTIGDEMNAVFLQRNAVPHWQNWRGLYRGLM
ALVSDTINALYQHGLR
FGENESH00000078270:
(SEQ ID NO: 8)
ATGACTTTGTGCTTTTTAAACACAAGCGCAGCGCTCGCTGATGAAGAGGG
CGATCCTCTGCCCACTGCTCTGATAAACAGTCTTGACCTAGCAGTGAATC
AGCCGGTGTCAGGTTCTGGCTTTTGTAAACTCAAACTGGCCAATGAGCAA
ACTGTTGTGACTCTCCAACAATTAGCAACAAGAGAACCCATGGGGGATGA
AGAGGAGGTGCAGGGCTTTCAGAGCACAGACCCACACGGGACAACTGTAT
GTGGAATGGCCCGACCAGAGATGGAAAGCAGAGTGGACGAACATAACTCT
GGCACGCCGAACAGCTGCAGGATGGAGGTGCTGCGTCAGGACGCCTGGCC
AAATGGCAGCATCATCCAGCCCTGCCATCGACGCCGAACCATTGCCACTC
AAACCAGCACTCTCTCTGCACCACTGCCCCACATCCCCTCACATGATGCC
TTCAGCTTGGACAGCGTCCAGCAGCAGGACAGTCTACTCAGGGACAATTC
AGGAACAGAACAGGAAGTGTCCAGGCCTCTTCCTCTGCCAGATCTGCTAG
CAGACAACCAGAGCTCCTCAGAGGAGTCCACGTCCAGCAGCAGCTCGACC
GCTGAGGAGGACCCCACACTGGAGGAGCAGGCTGTGGAGAGGGTGGCCGT
ACAACTGAGGACAATTGGGGACGAGATGAACGCTGTCTTCCTTCAGAGGA
ATGCCGTCCCGCACTGGCAGAACTGGAGAGGCCTGTACCGCGGGCTCATG
GCGCTGGTCTCGGACACCATCAATGCCCTCTACCAGCACGGCCTCAGATG
A

BLAST searching in the customized zebrafish sequence database (as described in Example 1(a)) did not identify FGENESH00000078270. TMHMM analysis did not predict a transmembrane domain for this sequence. FGENESH00000078270 did not match to the BH1, BH2, and BH4 HMMs. The sequence did match to the BH3 HMM with score of 13 and an e-value of 0.77. FGENESH00000078270 was similar to human Puma based on a BLAST alignment showing a 25% identity with an e-value of 2.1e1. Identification of the genes surrounding FGENESH00000078270 in the zebrafish genome indicated that FGENESH00000078270 shared a conserved syntenic relationship with human Puma.

The presence of a BH3-related region and an absence of BH1, BH2, and BH4-related regions and a transmembrane domain, combined with the sequence similarity and conserved synteny suggested strongly that FGENESH00000078270 was a member of the pro-apoptotic BH3-only subfamily of Bcl-2 proteins, and more specifically was orthologous to human Puma.

f. zBmf2 (FGENESH00000082230)

The zebrafish sequence FGENESH00000082230 was identified by feature-based database mining as described in Example 1(b) as a B2R protein. The amino acid sequence of FGENESH00000082230 and the nucleotide sequence encoding it are set forth below.

FGENESH00000082230:
(SEQ ID NO: 9)
MDDEEDEQLPRCCETPLRNKRSEKRDAHGGDVGQTAHRHASTQTAGSVLN
SARDADMAPFQGSQREASSLCRVVGARTAFRAPCGTGGLVSLTMGPGARG
GPRALFHGNAGFRAHFPALFEPALDGLQNAEQREQDEGRPEEKEEDRDAG
ISVEVQIGRKLREMGDQFQQEHLQLIILDETRYRI
FGENESH00000082230:
(SEQ ID NO: 10)
ATGGATGATGAGGAGGATGAACAGCTTCCTCGCTGCTGTGAAACGCCGCT
AAGAAACAAGCGCTCAGAGAAGAGAGACGCCCACGGTGGAGACGTGGGAC
AAACAGCTCACAGACACGCATCCACACAGACTGCTGGCTCTGTCCTCAAT
TCAGCGAGAGACGCAGATATGGCACCATTCCAGGGCTCACAGAGAGAAGC
ATCATCTCTTTGCAGAGTTGTGGGTGCTAGGACTGCATTCAGGGCTCCCT
GTGGAACTGGGGGCCTTGTGTCACTTACTATGGGCCCCGGAGCCCGGGGT
GGGCCAAGAGCACTTTTTCATGGAAACGCTGGATTTCGTGCACACTTCCC
TGCGCTGTTCGAACCCGCCCTGGATGGCTTACAAAACGCCGAACAGAGAG
AGCAGGACGAAGGCAGACCAGAAGAAAAAGAAGAGGATCGCGACGCAGGG
ATTAGCGTGGAGGTTCAGATTGGACGTAAATTACGTGAAATGGGGGATCA
GTTTCAGCAAGAGCATCTTCAGCTGATTATATTAGATGAAACCAGATACA
GGATTTAA

BLAST searching in the customized zebrafish sequence database (as described in Example 1(a)) did not identify FGENESH00000082230. TMHMM analysis did not predict a transmembrane domain for this sequence. FGENESH00000082230 did not match to the BH1, BH2, and BH4 HMMs. The sequence did match to the BH3 HMM with score of 18.6 and an e-value of 0.02. FGENESH00000082230 was similar to human Bmf based on BLAST alignment (41% identity over 42% of the molecule with an e-value of 2.1e1). The alignment also showed that FGENESH00000082230 contained a putative dynein light chain binding domain, similar to human Bmf (Day et al., Biochem J. 377:597-605 (2004)). Identification of the genes surrounding FGENESH00000082230 in the zebrafish genome indicated that FGENESH00000082230 shared a conserved syntenic relationship with human Bmf as well. The presence of a BH3-related region and an absence of BH1, BH2, and BH4-related regions and a transmembrane domain, combined with the sequence similarity and conserved synteny suggested strongly that FGENESH00000082230 was a member of the pro-apoptotic BH3-only subfamily of Bcl-2 proteins, and more specifically was orthologous to human Bmf.

Another zebrafish gene homologous to human Bmf had already been identified in zebrafish (Coultas et al., Cell Death Diff. 9: 1163-1166 (2002); accession number BI891121). That gene did not share conserved synteny with human Bmf, though, unlike FGENESH00000082230. The similarity between FGENESH00000082230 and B1891121 was 43% over 27% of the molecule. Accordingly, FGENESH00000082230 was designated zBmf2 to differentiate it from that earlier-identified gene.

TABLE 2
Characteristics of putative Bcl-2 family members in zebrafish

| | | | | % | % | Human | |
|---|---|---|---|---|---|---|---|
| Assignment | Protein accession number | e-value | Score | identity | coverage | gene | Syntenic? |
| zBim | FGENESH00000065416 | 9e−7 | 55 | 32 | 47 | Bim | Y |
| zBak | ENSDARP00000040899 | 4e−5 | 50 | 25 | 86 | Bak | N |
| | | 1e−14 | 81 | 33 | 51 | Bax | Y |
| zBax | ENSDARP00000023687 | 2e−6 | 54 | 29 | 68 | Bak | N |
| | | 2e−47 | 189 | 51 | 98 | Bax | N |
| zBik | ENSDARP00000066976 | 1.41e4 | 20 | 47 | 12 | Bik | Y |
| zPuma | FGENESH00000078270 | 2.1e1 | 30 | 25 | 49 | Puma | Y |
| zBmf2 | FGENESH00000082230 | 0.011 | 42 | 41 | 42 | Bmf | Y |
| zBad2 | XP_693331 | 8e−12 | 64 | 33 | 72 | Bad | N |

g. General Observations Regarding Zebrafish B2R Genes

Overall, the previously known and herein identified zebrafish homologs of the Bcl-2 family genes strongly resembled their human counterparts (Table 3), with the multi-domain Bcl-2 family members having the highest degree of similarity (FIG. 3A). The zebrafish BH3-only proteins were the most divergent from the human genes (FIG. 3A), ranging from 27% (zPuma) to 46% (zBad and zBmf2) overall similarity (see Table 3). However, the BH3-only proteins are known to be the most divergent of the vertebrate Bcl-2 subfamilies. A much higher degree of similarity was observed within the BH3 domain itself, ranging from 66-79% (Table 3; FIG. 3B).

TABLE 3
Comparison of zebrafish and human B2R proteins

| | | | | | | |
|---|---|---|---|---|---|---|
| Zebrafish | Mammalian | % | | Fish | Human | |
| gene | homolog | homology | Accession no. | chromosome | chromosome | Syntenic? |

Pro-survival

| | | | | | | |
|---|---|---|---|---|---|---|
| zBlp1 | Bcl-x_{L} | 67% | AF317837 | 5 | 20q11.21 | Yes |
| zBlp2 | Bcl-2 | 57% | NM_001030253.1 | 24 | 18q21.33 | No |
| zMcl-1a | Mcl-1 | 43% | NM_131599 | 19 | 1q21.2 | Yes |
| zMcl-1b | Mcl-1 | 38% | NM_194394.2 | 16 | 1q21.2 | Yes |
| zNR13 | Boo/ | 30% | AF441285 | 18 | 15q21.2 | Yes |
| | DIVA | | | | | |

Pro-apoptotic BH3-only

| | | | | | | |
|---|---|---|---|---|---|---|
| zBad1 | Bad | 46% | BC097099.1 | 7 | 11q13.1 | Yes |
| | | (79%) | | | | |
| zBad2 | Bad | 35% | XP_693331 | 21 | 11q13.1 | Yes |
| | | (66%) | | | | |
| zBid | Bid | 30% | XM_698543.1 | 18 | 22q11.21 | No |
| | | (66%) | | | | |
| zBik | Bik | 38% | ENSDARP0000006697 | 4 | 22q13.2 | Yes |
| | | (66%) | | | | |
| zBmf1 | Bmf | 46% | XM_695596.1 | 23 | 15q15.1 | No |
| | | (72%) | | | | |
| zBmf2 | Bmf | 38% | FGENESH00000082230 | 20 | 15q15.1 | Yes |
| | | (66%) | | | | |
| zNoxa | Noxa | 27% | DV595521.1 (EST) | 19 | 18q21.32 | No |
| | | (66%) | | | | |
| zPuma | Puma | 86% | FGENESH00000078270 | 16 | 19q13.32 | Yes |
| zBim | Bim | ND | FGENESH00000065416 | 13 | 2q13 | ND |

Pro-apoptotic multidomain

| | | | | | | |
|---|---|---|---|---|---|---|
| zBak | Bak | 41% | ENSDARP00000040899 | 3 | 6p21.31 | to hBax |
| zBax | Bax | 73% | ENSDARP00000023687 | ND | 19q13.33 | No |
| zBok1 | Bok | 81% | NM_001003612.1 | ND | 2q37.3 | ND |
| zBok2 | Bok | 74% | BC053218.1 | 2 | 2q37.3 | Yes |

ND indicates not determined. The parenthetical percentages indicate the % homology within the BH3 domain only.

### Example 3

### Experimental Validation of Bcl-2 Candidate Genes

The studies in Examples 1 and 2 demonstrated that the above-identified proteins shared homology with certain Bcl-2 family members and were likely members of the intrinsic apoptotic pathway. To confirm those assignments, the function of each protein was assessed in zebrafish.

a. Expression Patterns of Zebrafish B2R Genes

To assess the normal expression of the previously known and herein identified zebrafish B2R proteins, the mRNA encoding each protein was analyzed by RT-PCR at specific developmental stages and in specific adult tissues (FIGS. 3C and 3D). Adult Tubingen long-fin fish were obtained from the Zebrafish International Resource Center. Fish were maintained according to the Zebrafish Book (Westerfield, The Zebrafish Book. A guide for the laboratory use of zebrafish (Danio rerio). 4^{th} ed., Univ. of Oregon Press: Eugene (2000)). RNA was isolated from dechorionated wildtype zebrafish embryos (100 ng) at the indicated time points using QiaShredder (Qiagen) for 30 cycles followed by purification with the RNAeasy Mini kit (Qiagen) using DNaseI digestion according to the manufacturer's instructions. Adult tissues were isolated from an adult female zebrafish, and RNA (50 ng) was extracted from those tissues in a manner similar to the extraction from the embryonic tissue. GAPDH was included as a control for the amount of input RNA. The resulting products were resolved on 0.8% e-gels (Invitrogen) and photographed.

With the exception of zBik and zBok, all of the examined zebrafish B2R genes were expressed maternally (at the 1000 cell stage) (FIG. 3C). After initiation of zygotic transcription (approximately 5 hours post fertilization), zBad1, zBik, zBad, zBmf1, zNoxa, zBax, zMcl-1a, zMcl-1b, zBlp1, and zNR13 were expressed at fairly consistent levels (FIG. 3C). Conversely, the transcription of zBak, zBmf2, and zBlp2 decreased dramatically before increasing again later in development (FIG. 3C). zBok2 was strongly expressed maternally but was largely absent later in embryonic development (FIG. 3C).

Of the seven adult zebrafish tissues examined, the ovary displayed significant transcription of each of the genes except zBmf2 (FIG. 3D). In contrast, the liver displayed only weak expression of zBid, zMcl-1a, and zMcl-1b (FIG. 3D). Several of the zebrafish BH3-only genes were expressed in only a few tissues (FIG. 3D).

b. Apoptotic Effect of Putative Zebrafish B2R Proteins In Vivo

The ability of each of the previously known and herein identified zebrafish B2R genes to activate the intrinsic apoptotic pathway was assessed. Zebrafish cDNAs were directionally cloned into the expression vector pCS108 (Fletcher et al., Gene Expr. Patterns 5(2):225-30 (2004); http://tropicalis.berkeley.edu/home/genomic_resources/Ests/vectors/cs108.pdf., dated Aug. 13, 2001) using standard protocols. The primers used for the cloning were as follows (all 5′ to 3′):

zBak forward:
(SEQ ID NO: 65)
GGGGGTTGTTGTAAAGTACAGTGG
zBak reverse:
(SEQ ID NO: 66)
TCAGGTTTTCTGAAATCTCGAGACG
zBik forward:
(SEQ ID NO: 67)
ACTGTACTGAGACACATCACAGCAACA
zBik reverse:
(SEQ ID NO: 68)
TCAGCCTTCATACTCATTCCAG
zPuma forward:
(SEQ ID NO: 69)
CTAACTGAGTACTCATCTAATGAATTAACACCGCT
zPuma reverse:
(SEQ ID NO: 70)
TCATCTGAGGCCGTGCTGGTAGAG
zBmf1 forward:
(SEQ ID NO: 71)
ATGGATGAGGACGAGGATGAT
zBmf1 reverse:
(SEQ ID NO: 72)
TCACCTGCGGTTCTCTCTG
zBmf2 forward:
(SEQ ID NO: 73)
ATGGATGATGAGGAGGATGAAC
zBmf2 reverse:
(SEQ ID NO: 74)
TTAAATCCTGTATCTGGTTTCATCTA
zBad1 forward:
(SEQ ID NO: 75)
CTTCCACAACACTTCCACTGGATAC
zBad1 reverse:
(SEQ ID NO: 76)
TTCACTGTCAGTCACTCTGCGGGGC
zBlp1 forward:
(SEQ ID NO: 77)
GCCGAATTCCACCATGTCTTACTATAACCGAGAACTG
zBlp1 reverse:
(SEQ ID NO: 78)
GGCCTCGAGTCACAGGCGTTTCTGTGCAATGAGTCC
zBlp2 forward:
(SEQ ID NO: 79)
ATGGCTAACGAAATTAGC
zBlp2 reverse:
(SEQ ID NO: 80)
TCACTTCTGAGCAAAAAAGGC
zBok1 forward:
(SEQ ID NO: 81)
ATGGAGATGTTGCGCCGCT
zBok1 reverse:
(SEQ ID NO: 82)
TCATTTCTCTCTCAGCAGGTAGAAAAC
zBok2 forward:
(SEQ ID NO: 83)
ATGAACGTGTTCGCGCGCT
zBok2 reverse:
(SEQ ID NO: 84)
GCGCAAGTCTACTTCGTCAGGTA
zMcl-1a forward:
(SEQ ID NO: 85)
AAGGGGCTGCAGCTGGACG
zMcl-1a reverse:
(SEQ ID NO: 86)
AGCGGGGCTCAGGTCAGTACAG
zMcl-1b forward:
(SEQ ID NO: 87)
CATCTTCGATTGATTTGATT
zMcl-1b reverse:
(SEQ ID NO: 88)
ATAGTTTGAAAGTTCTGAATCC
zNR13 forward:
(SEQ ID NO: 89)
ATGTCCTGTTGGTTGAGGG
zNR13 reverse:
(SEQ ID NO: 90)
TCAGCGTACTAAGAGGAAGGT

The clone for zBad2 was obtained from Open Biosystems.

Capped synthetic mRNA for zBid, zBad1, zBmf1, zBmf2, zPuma, zBik, zNoxa, zBak, and zBax was generated by Ambion mMessage mMachine™ (Ambion) according to the manufacturer's directions, and purified over NucAway spin columns (Ambion). The resulting messenger RNA was diluted to the appropriate concentration (ranging from 0.11 mg/mL to 3.5×10⁻⁵ mg/mL) in 1× Danieu's solution including 0.2% phenol red. One to four-cell stage embryos were injected with 4.6 mL of the diluted mRNA solution using a Nanoliter 2000 (World Precision Instruments) microinjector. The faint pink cast to the blastomeres was residual phenol red from the injection solution.

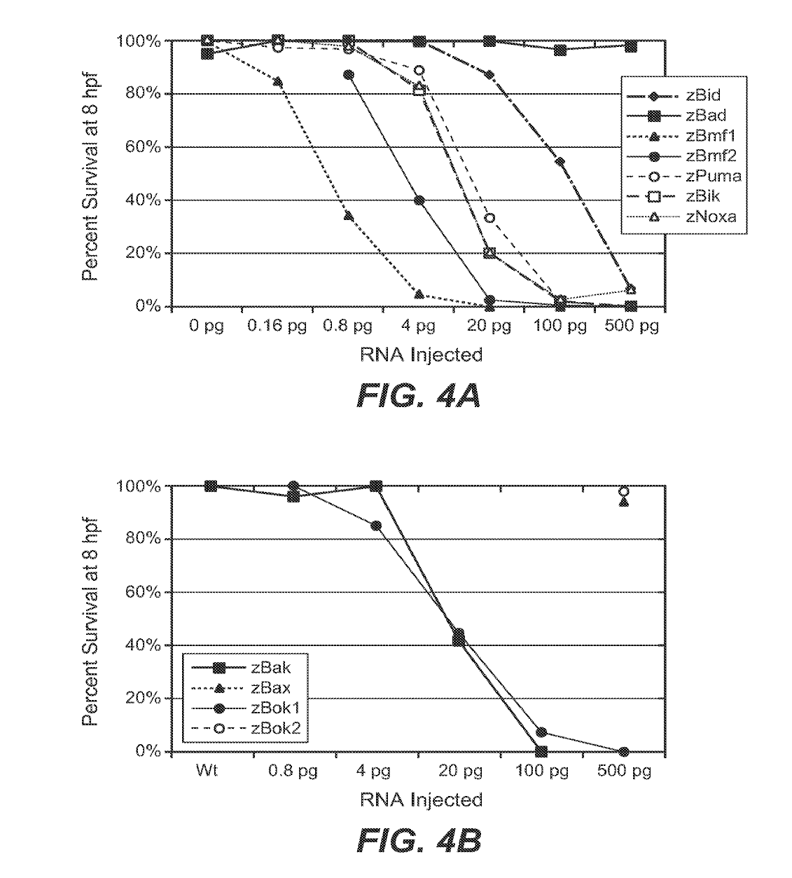

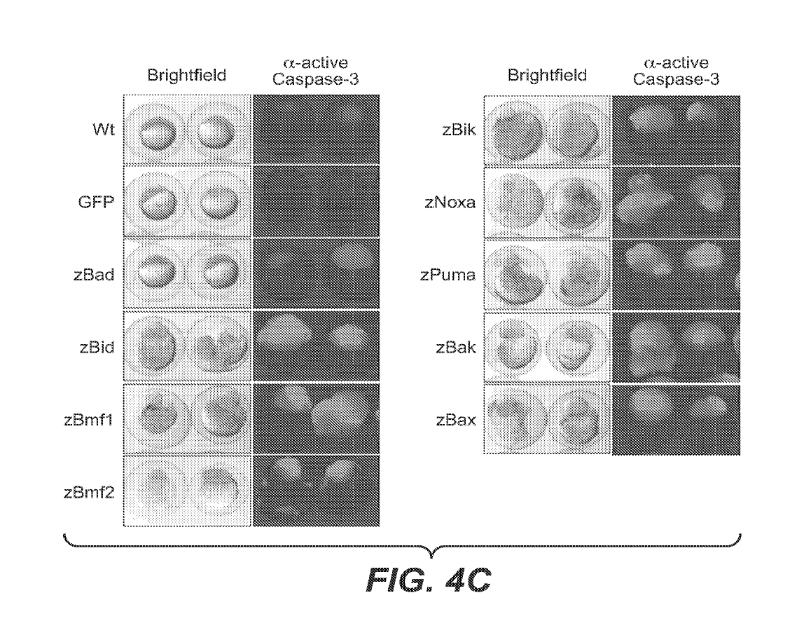

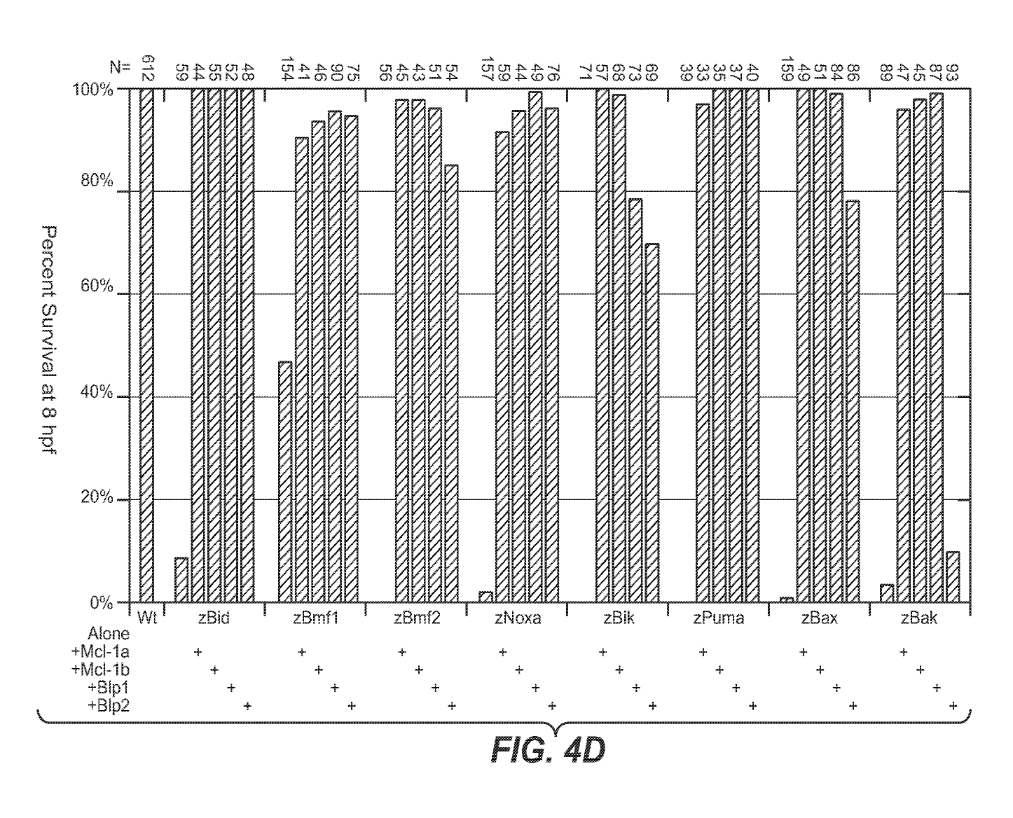

Injection of synthetic mRNA encoding most BH3-only proapoptotic proteins or multidomain pro-apoptotic Bcl-2 proteins resulted in dose-dependent apoptosis, characterized by disintegration of the blastomeres and yolk cell (FIGS. 4A and 4B). zBad, zBok1, and zBok2 did not induce significant apoptosis in early embryos injected with up to 500 pg of those synthetic mRNAs.

To confirm that ectopic expression of the zebrafish pro-apoptotic B2R genes killed embryos by engaging the apoptotic pathway, embryos were injected with a low dose of each synthetic mRNA, and the resulting degree of activation of the apoptosis effector caspase-3 was monitored by immunohistochemistry.

Zebrafish embryos were injected with 500 pg GFP, 500 pg zBad, 100 pg zBid, 20 pg zBik, 0.8 pg zBmf1, 4 pg of zBmf2, 20 pg zNoxa, 20 pg zPuma, 20 pg zBak, or 20 pg zBax at the 1-4 cell stage. Injected embryos were fixed at 10 hours post-fertilization in 4% paraformaldehyde in PBS for approximately 4 hours at room temperature or overnight at 4° C. Embryos were subsequently dehydrated in methanol for a minimum of two hours. After rehydration, embryos were washed with water, permeabilized in acetone for 7 minutes at −20° C., and washed again in water. Embryos were washed several additional times with PBS containing 0.5% Tween (PBST). The washed embryos were blocked for two hours at room temperature in 5% fetal bovine serum, 2 mg/mL BSA in PBST. Embryos were incubated with rabbit anti-activated caspase-3 antibody (Pharmingen) diluted 1:500 in blocking solution overnight at 4° C. Embryos were then washed several times in PBST before incubation with the secondary antibody, goat anti-rabbit Cy3 (Jackson Immunology) diluted 1:500 in blocking solution, at room temperature for two hours. Embryos were washed again with PBST before visualization with a Leica MZFL3 fluorescence microscope.

Compared to wildtype and mock-injected embryos, ectopic expression of the pro-apoptotic B2R proteins resulted in a dramatic increase in caspase-3 activity, indicating that ectopic expression of Bax-like and BH3-only B2R proteins initiated the apoptotic program in zebrafish embryos (FIG. 4C). The only exceptions were zBad, zBok1 and zBok2, which also did not initiate apoptosis when ectopically expressed (FIGS. 4A and 4C).

c. Rescue of Zebrafish B2R Protein-Induced Apoptosis

If ectopic expression of zebrafish pro-apoptotic B2R proteins in zebrafish embryos induced apoptosis via a mechanism similar to the mammalian apoptotic pathway, it should be possible to prevent lethality by co-expressing pro-survival B2R proteins. zMcl-1a, zMcl-1b, zBlp1, and zBlp2 were each cloned using the primers and protocol set forth in Example 3(b). Synthetic mRNA was created for each of zMcl-1a, zMcl-1b, zBlp1, and zBlp2 and co-injected into zebrafish embryos also according to the protocol described in Example 3(b), with the following B2R protein mRNA injection amounts: 500 pg of each pro-survival mRNA, 500 pg of zBid, 20 pg of zBmf1, 100 pg of zBmf2, 50 pg of zNoxa, 100 pg of zBik, 100 pg of zPuma, 100 pg of zBax, and 100 pg of zBak. The results are shown in FIG. 4D. Apoptosis induced by ectopic expression of zBid, zBik, zBmf1, zBmf2, zNoxa, zPuma, or zBax were rescued by co-expression with zMcl-1a, zMcl-1b, zBlp1, or zBlp2 (FIG. 4D). Ectopic expression of zBak, however, was rescued by co-expression with zMcl-1a, zMcl-1b, or zBlp1, but not by co-expression with zBlp2 (FIG. 4D), suggesting that zBak did not interact with zBlp2. Human Blp2 is a known homolog of Bcl-2. Previous studies had shown that human Bak does not interact with human Bcl-2. Thus, the inability of zBlp2 to rescue zBak-induced apoptosis further supported the designation of ENSDARP00000040899 as the zebrafish ortholog of human zBak.

d. Zebrafish B2R Gene Response to Gamma Radiation In Vivo

To investigate the role of the zebrafish B2R genes in response to an exogenous apoptotic stimulus, embryos were subjected to gamma irradiation. Gamma irradiation was known to trigger apoptosis in mammalian cells via the intrinsic pathway, resulting in an increase in caspase-3 activity in a p53-dependent manner (Gong et al., Cell Growth Differ. 10(7): 491-502 (1999)) The ability of the zebrafish pro-survival molecules to shield zebrafish embryos from gamma radiation-mediated apoptosis was therefore examined.

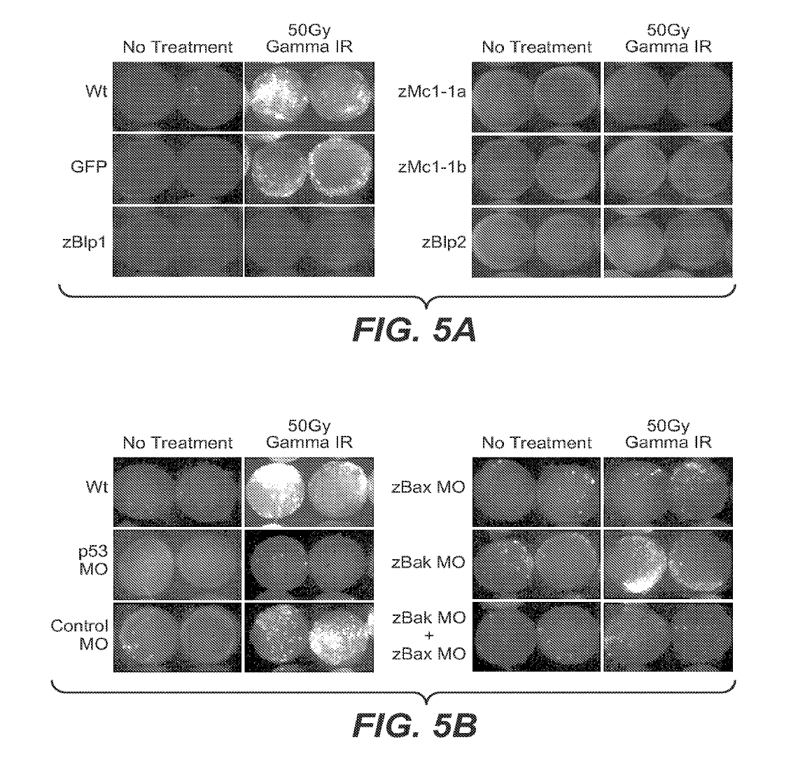

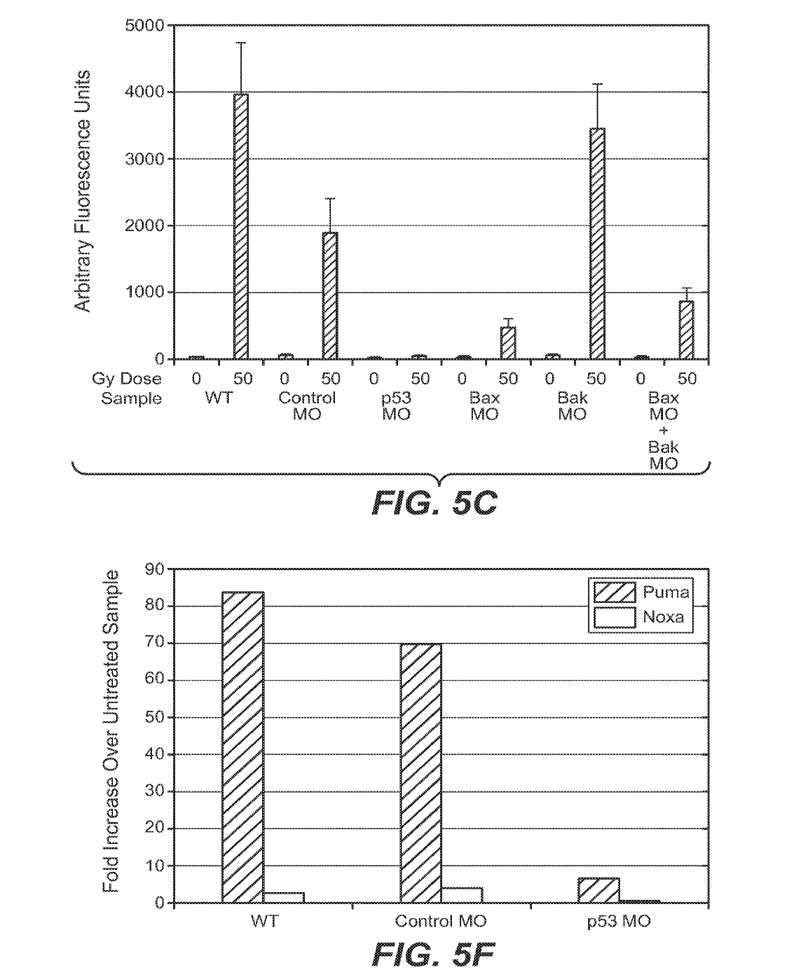

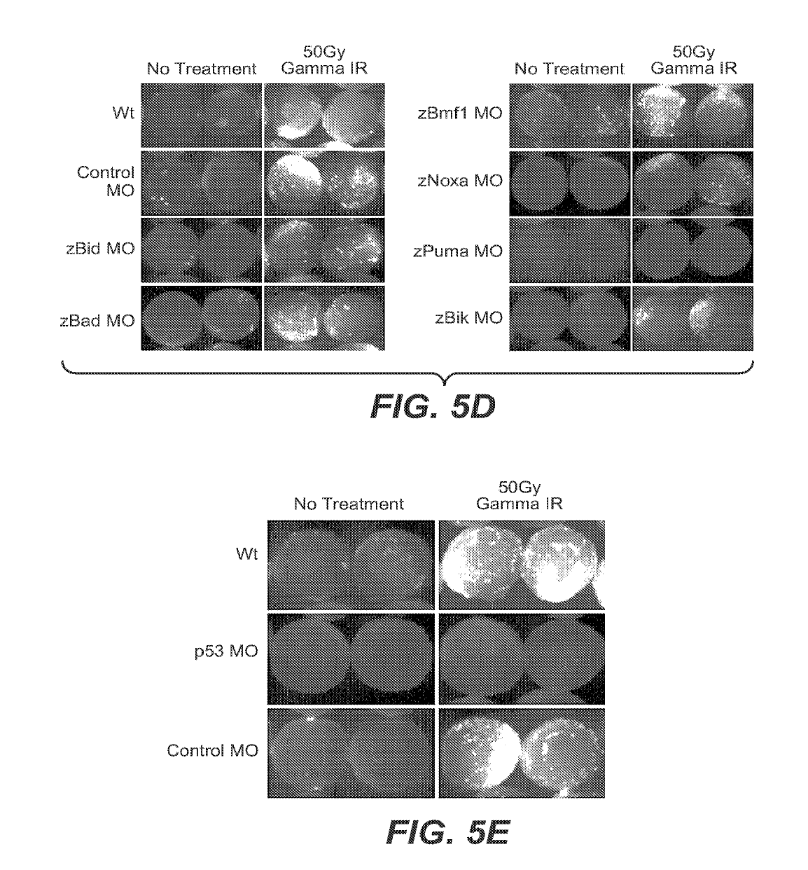

Embryos were irradiated at approximately 7 hours post fertilization in 1 mL of embryo media with a 50 Gy gamma irradiation dose. Embryos were subsequently moved to a tissue culture dish in a greater volume of embryo media and incubated at 28.5° C. until further analysis. Ecotopic expression of each of the zebrafish pro-survival B2R genes (zBlp1, zMcl-1a, zMcl-1b, and zBlp2), as described in Example 3(b) (modified to 500 pg injections) protected embryos from gamma irradiation-induced apoptosis (FIG. 5A). Thus gamma radiation triggered apoptosis in zebrafish embryos via the intrinsic pathway, similar to its effects in mammalian cells.

To determine which zebrafish B2R genes were responsible for mediating the apoptotic effects of gamma radiation on the zebrafish embryos, translational knockdowns of the pro-apoptotic B2R zebrafish genes were made using a morpholino approach. Morpholinos to each pro-survival B2R gene were selected based on their ability to abrogate cognate mRNA-mediated rescue of ectopically expressed zNoxa. Morpholinos for each pro-apoptotic B2R gene were selected based on their ability to rescue ectopic expression of the cognate mRNA. As morpholino efficacy could not be verified for zBok1, zBok2, or zBad (due to the fact that those genes did not induce apoptosis when ectopically expressed, see FIGS. 4A and 4C), those genes were not included in the knockdown analyses.

Morpholinos were designed around the translational start site of each transcript (Nasevicius and Ekker, Nat. Genet. 26: 216-220 (2000)), and obtained from GeneTools. Morpholino (“MO”) sequences were as follows (all 5′ to 3′):

| (SEQ ID NO: 91) | |
|---|---|
| zMcl-1a MO: | GCCTAAAATCCAAACTCAGAGCCAT |
| (SEQ ID NO: 92) | |
| zMcl-1b MO: | TGTCGTTGTTTCTTCCAGCGAACAT |
| (SEQ ID NO: 93) | |
| zBlp1 MO: | AGGTTGTTGCTCGTTCTCCGATGTC |
| (SEQ ID NO: 94) | |
| zBlp2 MO: | GTCATAGCTAATTTCGTTAGCCATG |
| (SEQ ID NO: 95) | |
| zBax MO: | TGAAAATAAGCGAACTGAAGAAGAC |
| (SEQ ID NO: 96) | |
| zBak MO: | ATTTTTCGGCTAAAACGTGTATGGG |
| (SEQ ID NO: 97) | |
| zBid MO: | GGTCAAAGTTCCTGTTGAAGTCCAT |
| (SEQ ID NO: 98) | |
| zBmf MO: | ACACATCATCCTCGTCCTCATCCAT |
| (SEQ ID NO: 99) | |
| zNoxa MO: | CTTTCTTCGCCATTTCAGCAAGTTT |
| (SEQ ID NO: 100) | |
| zPuma MO: | TGCTTTCCATCTCTGGTCGGGCCAT |
| (SEQ ID NO: 101) | |
| zBik MO: | CTACAAACAAGGACACAATGGTGGA |
| (SEQ ID NO: 102) | |
| p53 MO: | GCGCCATTGCTTTGCAAGAATTG |
| (SEQ ID NO: 103) | |
| control MO: | CCTCTTACCTCAGTTACAATTTATA |

The control morpholino was designed to restore normal human beta globin mRNA sequence messages containing the mutant beta thalassemia splice site, and was expected to have no effect in zebrafish embryos. The p53 morpholino was according to Langheinrich et al., Curr. Biol. 12: 2023-2028 (2002).

Morpholinos were diluted to 1 mg/mL in 1× Danieu's solution+0.2% phenol red. A total of either 4.6 ng or 9.2 ng of morpholino was injected. In experiments where a combination of two morpholinos was used, 4.6 ng of each morpholino was injected. An additional 4.6 ng of control morpholino was added to single morpholino injections when the experiment included comparison to a dual morpholino injection sample, such that each sample was injected with 9.2 ng of morpholino. 1-4 cell stage embryos were injected with 4.6 mL of diluted morpholino using a Nanoliter 2000 (World Precision Instruments) microinjector.

In most mammalian cell types, either Bax or Bak is required to transduce most apoptotic stimuli (Wei et al., Science 292: 727-730 (2001)). To determine whether zBax and zBak were functionally redundant in zebrafish, morpholinos directed against zBax and zBak were injected singly or pairwise into embryos, and the embryos were subsequently subjected to gamma irradiation. Translational knockdown of zBax was sufficient to protect the embryos from the effects of gamma radiation (FIGS. 5B and 5C). In a small percentage of clutches, knockdown of both zBax and zBak was required to abrogate gamma irradiation-induced Caspase-3 activation. Some caspase-3 activity remained in the irradiated embryos injected with zBax and zBak morpholinos (FIGS. 5B and 5C), possibly due to incomplete knockdown or the function of maternal proteins present in the embryo. zBax appeared to be primarily responsible for executing the apoptotic program in response to gamma irradiation.

Knockdown of translation of several BH3-only proapoptotic genes revealed that when zPuma translation was impaired, caspase-3 activation was dramatically reduced in response to gamma irradiation (FIG. 5D). zNoxa impairment also greatly reduced caspase-3 activation, but not as completely as zPuma inactivation. This effect mirrored the protective effect of p53 knockdown (FIG. 5E), and was supported by previous studies in mammalian cells indicating that Puma is the primary mediator of gamma irradiation-induced apoptosis (Erlacher et al., Blood 106: 4131-4138 (2005); Jeffers et al., Cancer Cell 4: 321-328 (2003)).

A quantitative PCR analysis was undertaken to better understand the actions of zNoxa and zPuma during gamma irradiation. Taqman analyses were performed using the 7500 Real Time PCR System (Applied Biosystems) according to the manufacturer's instructions. The primer and probe sequences for each gene are shown below (5′ to 3′):

zGAPDH forward:
(SEQ ID NO: 104)
TGCGTTCGTCTCTGTAGATGT
zGAPDH reverse:
(SEQ ID NO: 105)
GCCTGTGGAGTGACACTGA
zGAPDH probe:
(SEQ ID NO: 106)
TGTGTGTGTGTGTTAGTTTCTTTTGACAGTATTTG
zNoxa forward:
(SEQ ID NO: 107)
CGAACCTGTGACAGAAACTTG
zNoxa reverse:
(SEQ ID NO: 108)
CTGCGCGCACTCTACTACA
zNoxa probe:
(SEQ ID NO: 109)
CGGTTTGCTCTTTCTTCGCCATTTC
zPuma forward:
(SEQ ID NO: 110)
GAACACACGGGTTACAAAAGAC
zPuma reverse:
(SEQ ID NO: 111)
GAAAATTCCCAGAGTCTGTAAGTG
zPuma probe:
(SEQ ID NO: 112)
ACGAGTGCAGGCGCTCTCCTT

Embryos were collected approximately 30 minutes post-fertilization and maintained in embryo media for further analysis. Coding regions for each B2R gene were amplified by RT-PCR using the OneStep PT-PCR kit (Qiagen) according to the manufacturer's directions. Multiple PT-PCR products were sequenced to verify the correct coding sequence. Each amplified sequence was cloned by Topo TA (Invitrogen) cloning into the plasmid pCRII (Invitrogen). Embryos were irradiated with 50 Gy at seven hours post fertilization, and the RNA was collected at 10 hours post fertilization.

The quantitative PCR analysis revealed that while zNoxa transcription was upregulated 3-4 fold, zPuma transcription was upregulated almost 100-fold in response to gamma irradiation (FIG. 5F). None of the other zebrafish BH3-only genes showed an increase in transcription in response to gamma irradiation. Upregulation of zPuma was p53-dependent (FIG. 5F), such that knockdown of p53 decreased zPuma upregulation. This suggested that in the zebrafish gamma irradiation induced p53 activity, which in turn transcriptionally upregulated zPuma. Puma upregulation was known in mammalian systems to activate Bax and Bak (Liu et al., Biochem. Biophys. Res. Commun. 310(3): 956-62 (2003)), correlating with the above data suggesting that zBax is critical to induction of apoptosis in response to gamma irradiation. Thus, gamma irradiation of zebrafish embryos triggered the intrinsic apoptotic pathway, mediated in particular by p53, zPuma, and zBax.

e. Knockdown of Zebrafish Pro-Survival B2R Proteins During Normal Development

Having established that the intrinsic apoptotic pathway was present and functional in zebrafish, the zebrafish intrinsic pathway was compared to the known mammalian intrinsic pathway system. Zebrafish pro-survival B2R genes were subjected to morpholino knockdown, and the resulting effects on the developing embryo were monitored. Morpholinos were directed against the translational start site of zMcl-1a, zMcl-1b, and zBlp2 according to the methodology described in Example 3(d).

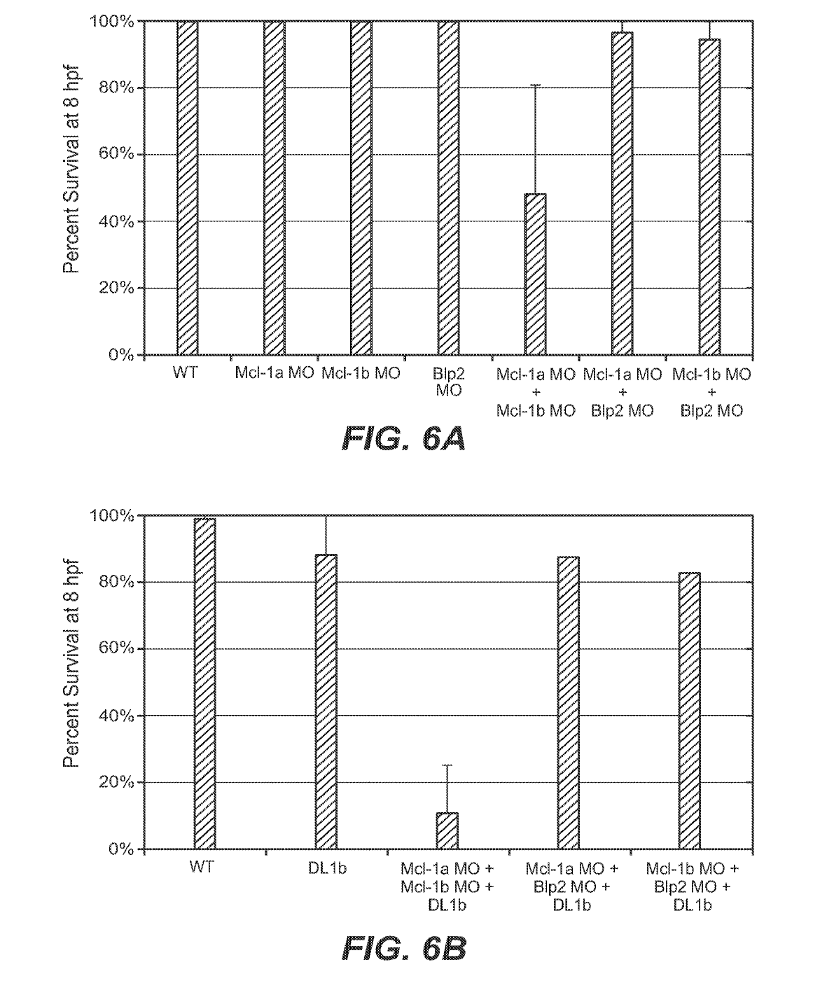

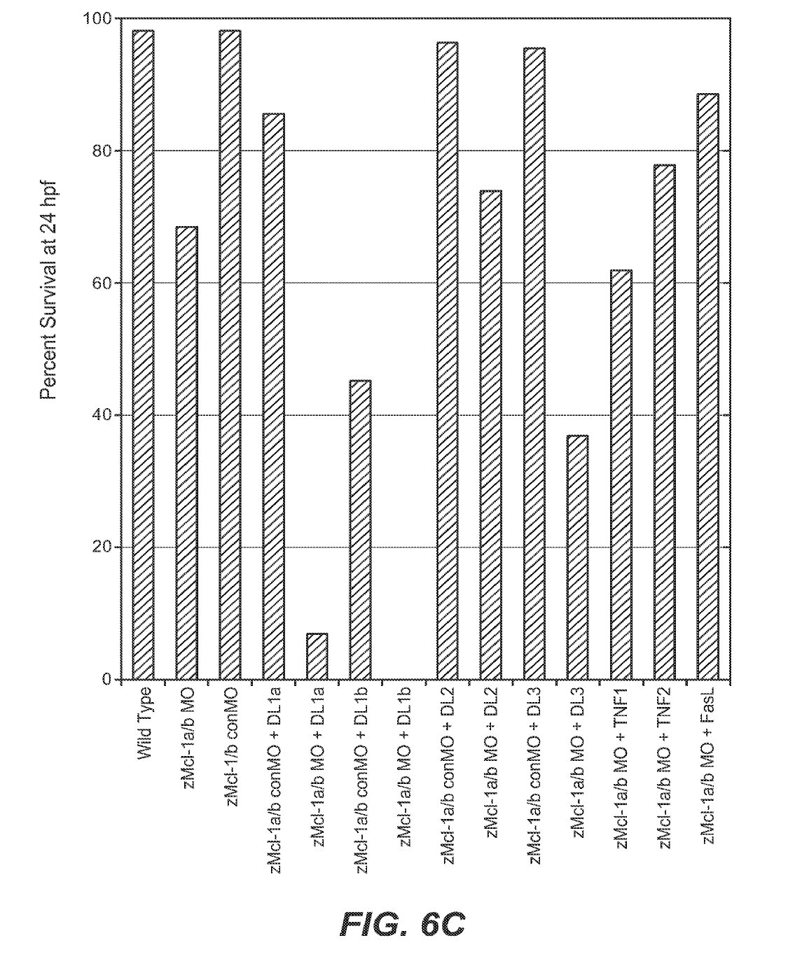

Knockdown of zBlp2 had no obvious effect on early zebrafish development (FIG. 6A). The gross morphology of the zBlp2 knockdown fish was normal. There was no increase in caspase-3 activity as a result of zBlp2 knockdown. Similarly, knockdown of either zMcl-1a or zMcl-1b had no apparent effect on survival of fish embryos. However, knocking down both zMcl-1a and zMcl-1b resulted in a variable but significant decrease in viability by 8 hours post fertilization (FIG. 6A). Pairwise knockdowns of either of the zMcl genes in combination with zBlp2 had no impact on survival (FIG. 6A). Thus, only impairing transcription of both copies of zMcl-1 significantly affected the survival of injected zebrafish embryos. Notably, previous studies had demonstrated that knockouts of mammalian Mcl-1 in mice are pre-implantation lethal (Rinkenberger et al., Genes Dev. 14: 23-27 (2000)).

f. Zebrafish Pro-Survival B2R Proteins and Apo2L Signaling

Zebrafish embryos injected with both zMcl-1a and zMcl-1b morpholinos displayed a significant range of viability (Example 3(e)). This range of viability appeared to vary with the overall “health” of the clutch. One possible explanation for the variability was that zMcl-1a/b might protect early zebrafish embryos from endogenous or environmental apoptotic stimuli. Mcl-1 had previously been implicated in mediating sensitivity to Apo2L-induced apoptosis in several cell lines (Henson et al., J. Cell Biochem. 89:1177-1192 (2003); Taniai et al., Cancer Res. 64: 3517-3524 (2004); Wirth et al., Cancer Res. 65: 7393-7402 (2005); Kobayashi et al., Gastroenterology 128: 2054-2065 (2005)). Thus, the effect of zMcl-1a and zMcl-1b knockdown on the Apo2L-induced extrinsic apoptotic pathway was investigated.

Knockdowns of zMcl-1a and zMcl-1b were performed as described in Example 3(e). Zebrafish Apo2L homolog zDL1b and other TNF-related genes were cloned as described in Example 3(b) using the following primer sequences:

zDL1a forward:
(SEQ ID NO: 113)
ACCATGATGGTCCCGGCGAACAGCCGC
zDL1a reverse:
(SEQ ID NO: 114)
ACTTTACAGATCCAATCGGAAAGCTCC
zDL1b forward:
(SEQ ID NO: 115)
ATCATGGTGCAGCCTAAAAATCGT
zDL1b reverse:
(SEQ ID NO: 116)
CCCTCAGAGGTCAAACAGGAAGGC
zDL2 forward:
(SEQ ID NO: 117)
CACGCGATGGTCAGCATGACAAGC
zDL2 reverse:
(SEQ ID NO: 118)
TACTGACTAGCTCACCAGAAATGC
zDL3 forward:
(SEQ ID NO: 119)
ACCATGACATCCAACCTTCCTATCGGT
zDL3 reverse:
(SEQ ID NO: 120)
AGTTTATTTAATCATGAATGCCCCAAA
zFasL forward:
(SEQ ID NO: 121)
ATGAGTGCTAACTTCGGCCACTCG
zFasL reverse:
(SEQ ID NO: 122)
TCAGTGGATCTTAAAGAGGCCGAA
zTNF1 forward:
(SEQ ID NO: 123)
GCAACCATGAAGCTTGAGAGTCGGGCGTTT
zTNF1 reverse:
(SEQ ID NO: 124)
TTTCGTTCACAAACCAAACACCCCAAAGAA
zTNF2 forward:
(SEQ ID NO: 125)
ATGGTGAGATACGAAACAACATTA
zTNF2 reverse:
(SEQ ID NO: 126)
ATTAAATCACAACGCGAACACCCCGAAGAA.

Synthetic zDL1b mRNA was produced and injected into zebrafish embryos as described in Example 3(b).

In wildtype embryos, ectopic expression of zebrafish Apo2L ortholog zDL1b had minimal effect on early embryonic viability (FIG. 6B). However, when knockdown of zMcl-1a and zMcl-1b was combined with ectopic expression of zDL1b, embryos rapidly underwent massive apoptotic death (FIG. 6B). The effect was specific to zMcl-1a and zMcl-1b, because knockdown of either zMcl-1a or zMcl-1b in conjunction with zBlp2 did not increase the sensitivity to zDL1b-induced apoptosis (FIG. 6B).

Differing effects were obtained with other Apo2L/TNF-related molecules aside from zDL1b (see FIG. 6C). For example, ectopic expression of zDL1a in conjunction with the dual translational knockdown of zMcl-1a and zMcl-1b resulted in nearly as great a reduction in survival of zebrafish embryos as with zDL1b (FIG. 6C). The dual knockdown also markedly decreased survival when zDL3 was ectopically expressed (FIG. 6C). Significantly less reduction in percent survival was obtained when zDL2, zTNF1, or zTNF2 expression was paired with the dual zMcl-1a/b translational knockdowns (FIG. 6C). zFasL did not induce apoptosis, either alone or in combination with the knockdown of zMcl-1a and zMcl-1b. The expression of receptors for zTNF1, zTNF2, and zFasL on embryonic cells has not yet been established.

Thus, zMcl-1a and zMcl-1b together protected zebrafish embryos from zDL1a and zDL1b (Apo2L)-induced apoptosis, and had a lesser, but still significant protective effect from zDL3-induced apoptosis.

What is claimed:

1. A polypeptide having an amino acid sequence selected from SEQ ID NOs: 1, 5, 7, and 9 or a variant thereof, wherein the polypeptide is a zebrafish Bcl-2-related (B2R) multidomain pro-apoptotic polypeptide or a zebrafish B2R BH3-only pro-apoptotic polypeptide.

2. A polynucleotide having a nucleotide sequence selected from SEQ ID NOs: 2, 6, 8, and 10 or a variant thereof, wherein the polynucleotide encodes a zebrafish B2R BH3-only pro-apoptotic polypeptide.

3. A transgenic zebrafish, wherein one or more polynucleotide encoded by a nucleotide sequence selected from SEQ ID NOs: 2, 6, 8, and 10 is deleted.

4. A transgenic zebrafish, wherein the expression of one or more polynucleotides selected from SEQ ID NOs: 2, 6, 8, and 10 is modulated relative to the expression of the one or more polynucleotides in a wild-type zebrafish.

5. The transgenic zebrafish of claim 4, wherein the expression is increased.

6. The transgenic zebrafish of claim 4, wherein the expression is decreased.

7. A transgenic zebrafish, wherein one or more polypeptides selected from SEQ ID NOs: 1, 5, 7, and 9 are not expressed.

8. A transgenic zebrafish, wherein the expression of one or more polypeptides selected from SEQ ID NOs: 1, 5, 7, and 9 is modulated relative to the expression of the one or more polypeptides in a wild-type zebrafish.

9. The transgenic zebrafish of claim 8, wherein the expression is increased.

10. The transgenic zebrafish of claim 8, wherein the expression is decreased.

11. A transgenic zebrafish, wherein one or more endogenous zebrafish pro-apoptotic (ZPA) genes is replaced with a variant ZPA gene or with a ZPA gene counterpart from another organism.

12. The transgenic zebrafish of claim 11, wherein the counterpart is mammalian.

13. The transgenic zebrafish of claim 11, wherein the counterpart is human.

14. The transgenic zebrafish of claim 11, wherein all of the endogenous ZPA genes are replaced with ZPA gene counterparts from another organism.

15. A transgenic zebrafish, wherein one or more endogenous intrinsic apoptotic pathway genes is replaced with an intrinsic apoptotic pathway gene variant or an intrinsic apoptotic pathway gene counterpart from another organism.

16. The transgenic zebrafish of claim 15, wherein the counterpart is mammalian.

17. The transgenic zebrafish of claim 15, wherein the counterpart is human.

18. The transgenic zebrafish of claim 15, wherein all of the endogenous intrinsic apoptotic pathway genes are replaced with intrinsic apoptotic pathway gene counterparts from another organism.

19. The transgenic zebrafish of claim 11 or claim 15, wherein the one or more endogenous ZPA genes are selected from SEQ ID NOs: 2, 6, 8, and 10.

20. A model system for apoptosis, comprising the zebrafish of any of claims 3-19.

21. An in vitro model system for apoptosis, comprising at least one polynucleotide encoded by a nucleotide sequence selected from SEQ ID NOs: 2, 6, 8, and 10.

22. The model system of claim 20 or claim 21, wherein the model system for apoptosis is a model system for the intrinsic apoptotic pathway.

23. An in vitro apoptosis model system, comprising one or more polypeptides selected from SEQ ID NOs: 1, 5, 7, and 9.

24. The apoptosis model system of claim 23, wherein the model system is a model system for the intrinsic apoptotic pathway.

25. The apoptosis model system of claim 23, wherein the model system is a model system for the extrinsic apoptotic pathway.

26. A method of identifying a compound that binds to a ZPA polypeptide, comprising contacting a ZPA polypeptide with a compound and determining whether the compound binds to the ZPA polypeptide.

27. A method for identifying a compound which modulates the activity of a ZPA polypeptide, comprising contacting a ZPA polypeptide with a compound and determining whether the compound modulates the activity of the ZPA polypeptide.

28. A method for identifying an agent for reducing or preventing apoptosis, comprising administering at least one agent to a zebrafish and determining whether apoptosis is reduced or prevented.

29. The method of claim 28, further comprising determining the presence or amount of apoptosis in the zebrafish prior to administering the at least one agent.

30. The method of claim 28, further comprising stimulating apoptosis in the zebrafish prior to administering the at least one agent.

31. The method of claim 28, wherein the agent reduces or prevents apoptosis through the intrinsic apoptotic pathway.

32. The method of claim 28, wherein the agent reduces or prevents apoptosis through the extrinsic apoptotic pathway.

33. The method of claim 28, wherein the expression and/or activity of one or more ZPA proteins in the zebrafish is increased relative to the expression or activity of the one or more ZPA proteins in a wild-type zebrafish.

34. The method of claim 28, wherein one or more ZPA proteins is not expressed in the zebrafish.

35. The method of claim 28, wherein the expression and/or activity of one or more ZPA proteins is reduced in the zebrafish relative to the expression and/or activity of the one or more ZPA proteins in a wild-type zebrafish.

36. The method of claim 28, wherein the agent is selected from an antibody, an antibody fragment, an aptamer, and a small molecule.

37. The method of claim 28, wherein the zebrafish is a larval zebrafish.

38. The method of claim 28, wherein the determining step comprises microscopic examination of cell viability.

39. The method of claim 28, wherein the determining step comprises determining caspase activation.

40. A method for identifying an agent for initiating and/or stimulating apoptosis, comprising administering at least one agent to a zebrafish and determining whether apoptosis is initiated or increased.

41. The method of claim 40, further comprising determining the presence or amount of apoptosis in the zebrafish prior to administering the at least one agent.

42. The method of claim 40, further comprising preventing and/or decreasing apoptosis in the zebrafish prior to administering the at least one agent.

43. The method of claim 40, wherein the agent initiates and/or stimulates apoptosis through the intrinsic apoptotic pathway.

44. The method of claim 40, wherein the agent initiates and/or stimulates apoptosis through the extrinsic apoptotic pathway.

45. The method of claim 40, wherein the expression and/or activity of one or more ZPA proteins in the zebrafish is increased relative to the expression or activity of the one or more ZPA proteins in a wild-type zebrafish.

46. The method of claim 40, wherein one or more ZPA proteins is not expressed in the zebrafish.

47. The method of claim 40, wherein the expression and/or activity of one or more ZPA proteins is reduced in the zebrafish relative to the expression and/or activity of the one or more ZPA proteins in a wild-type zebrafish.

48. The method of claim 40, wherein the agent is selected from an antibody, an antigen-binding antibody fragment, an aptamer, and a small molecule.

49. The method of claim 40, wherein the zebrafish is a larval zebrafish.

50. The method of claim 40, wherein the determining step comprises microscopic examination of cell viability.

51. The method of claim 40, wherein the determining step comprises determining caspase activation.

52. A method of treating an apoptosis-related disorder, comprising administering to a patient at least one polypeptide encoded by an amino acid sequence selected from SEQ ID NOs: 1, 5, 7, and 9.

53. A method of treating an apoptosis-related disorder, comprising administering to a patient an antagonist of at least one polypeptide encoded by an amino acid sequence selected from SEQ ID NOs: 1, 5, 7, and 9.

54. The method of claim 53, wherein the antagonist is selected from an aptamer, an antibody, an antigen-binding antibody fragment, and a small molecule.

55. A method of treating an apoptosis-related disorder, comprising administering to a patient an agonist of at least one polypeptide encoded by an amino acid sequence selected from SEQ ID NOs: 1, 5, 7, and 9.

56. A method of treating an apoptosis-related disorder, comprising administering to a patient at least one polypeptide selected from the group of polypeptides encoded by the polynucleotide sequences of SEQ ID NOs: 2, 6, 8, and 10.

57. The method of any of claims 52-56, wherein the apoptosis-related disorder is selected from a cell proliferative disorder, a viral apoptosis disorder, an autoimmune disorder, a hematologic disorder, and a neurological disorder.

58. A method for identifying an agent for preventing or decreasing apoptosis, comprising contacting at least one polypeptide encoded by an amino acid sequence selected from SEQ ID NOs: 1, 5, 7, and 9 with the agent and determining the ability of the agent to block or decrease activity of the at least one polypeptide.

59. A method for identifying an agent for initiating or stimulating apoptosis, comprising contacting at least one polypeptide encoded by an amino acid sequence selected from SEQ ID NOs: 1, 5, 7, and 9 with the agent and determining the ability of the agent to stimulate or increase activity of the at least one polypeptide.

60. A method for identifying an agent for preventing or decreasing apoptosis, comprising contacting a cell comprising at least one polynucleotide encoded by a nucleotide sequence selected from SEQ ID NOs: 2, 6, 8, and 10 with the agent and determining the ability of the agent to prevent or decrease expression of the at least one polynucleotide.

61. A method for identifying an agent for initiating or stimulating apoptosis, comprising contacting a cell comprising at least one polynucleotide encoded by a nucleotide sequence selected from SEQ ID NOs: 2, 6, 8, and 10 with the agent and determining the ability of the agent to stimulate or increase expression of the at least one polynucleotide.

62. A composition for increasing apoptosis, comprising a polypeptide encoded by an amino acid sequence selected from SEQ ID NOs: 1, 5, 7, and 9, or an agonist thereto.

63. A composition for reducing or preventing apoptosis, comprising an antagonist of one or more of SEQ ID NOs: 1, 5, 7, and 9.

64. The composition of claim 63, wherein the antagonist is selected from an antibody, an antigen-binding antibody fragment, an aptamer, and a small molecule.

65. A composition for reducing or preventing apoptosis, comprising an agent that reduces or inhibits expression of one or more of SEQ ID NOs: 2, 6, 8, and 10.

66. The composition of any of claims 62 to 65, further comprising a pharmaceutically-acceptable carrier.

67. A method of treating an apoptosis-related disorder in a subject in need of treatment, comprising administering to the subject the composition of any of claims 62 to 65.

68. The method of claim 67, wherein the apoptosis-related disorder is selected from a cell proliferative disorder, a viral apoptosis disorder, an autoimmune disorder, a hematologic disorder, and a neurological disorder.

69. A method of detecting the presence or severity of an apoptosis-related disorder, or a predisposition thereto, comprising detecting the presence or amount of a ZPA polypeptide homolog in a subject at risk for developing the apoptosis-related disorder.

70. A method of detecting the presence or severity of an apoptosis-related disorder, or a predisposition thereto, comprising detecting the presence or amount of expression of a ZPA polynucleotide homolog in a subject at risk for developing the apoptosis-related disorder.

71. A kit comprising a composition according to any of claims 62 to 65 and instructions for use.

72. A kit comprising an in vitro apoptosis model system of any of claims 21-25 and instructions for use.

73. An article of manufacture comprising: (a) a composition according to any of claims 62 to 65, (b) a container containing said composition; and (c) a label affixed to the container, or a package insert included in the container referring to the use of the composition in the treatment of an apoptosis-related disorder.


SEQUENCE LISTING (126 sequences)

SEQ ID NO: 1 - protein, 207 aa, Danio rerio
MACEASQDDQIGEALLIGVVRQELMEVMEVTEGNAAPPALPEAKPISNSQDQILVQQLANTIKVIGDKLDQDQAFNDMIDGLVKVADKSSFWKLVEKVFTDGQINWGRIIVLFYSVGKLSAKMVVARLPRIVSDILSLSLDYFKRNLLQWIRTVGGWMNSIPALACFSVDQFSGSSMRKYSPYVGVVFAFTGGLLLGGFIVSRFQKT

SEQ ID NO: 2 - DNA, 624 bp, Danio rerio
atggcttgtgaagcctcacaggatgatcagattggagaggcactcttaataggggtagtaaggcaggagctaatggaggtgatggaggtgactgaaggaaatgcagctcctccagctcttcctgaagctaaaccaataagcaacagccaggaccagattctggttcagcagctggcgaacacaatcaaagtgatcggtgacaaactcgaccaggatcaagcatttaacgacatgatcgatggcttagtaaaggtagctgataaaagcagtttctggaaacttgtggaaaaggtgttcacagatggccagatcaactggggcagaattatcgtgctgttttattctgttggaaaactgtcagccaagatggtcgtcgctcgcctacccagaattgtttcagatattttatcattaagtcttgattacttcaaaaggaatctgttgcagtggattcgcacagtaggaggatggatgaacagtatccctgcactggcctgtttctctgttgaccaattttctggttcttcaatgagaaaatattctccttacgttggagttgtgtttgccttcactggtggcctactgctgggtggcttcatcgtctcgagatttcagaaaacctga

SEQ ID NO: 3 - protein, 145 aa, Danio rerio
MENTSHDHQDDSSTLDEKERSHLKGTIKNHGQHQDRTSANISPQGRVRLYSESQVYTVSRWQDTETQDGASVEENGDGLPFRGRSQSAPAALWKAKKYGRQLRRMSDEFDTWLDKGEVKRANSQKQTYRGWFSFLWSPKEEEGRE

SEQ ID NO: 4 - DNA, 438 bp, Danio rerio
atggagaacacctcgcatgaccatcaagatgattccagcaccttggatgaaaaagagagatcacatctgaaagggacaatcaagaaccatggacaacatcaggatcgaacatcggccaacatttctcctcaagggcgtgtgcggctctattcggaatctcaagtgtatacagtcagccgctggcaggacacagagacccaggatggagcatcggtggaggagaacggagatggacttccattcaggggtcgttctcaatcagcacctgctgcactgtggaaagcaaaaaagtatggccgtcagttgaggagaatgagcgatgaattcgacacatggctcgataaaggggaggtcaagagagcgaacagccagaaacagacctaccgaggatggttttcgttcctctggagtcccaaagaagaagagggcagagaatga

SEQ ID NO: 5 - protein, 154 aa, Danio rerio
MVEETRQQKNATTLQAGPAEVDHSNLYAFNMRVTQTIGRQLAQIGDEMDNKWRQEPPVPWQNLNFGIYPYVLSRRVFSGRILANLWGSKIMPIFRTSWLLPQLQNGCQEARKWAAWVSNLHVSDWSRSTTYTLASALLLVTVSIFLVNWNEYEG

SEQ ID NO: 6 - DNA, 465 bp, Danio rerio
atggtggaagaaactagacagcagaaaaacgccacaaccctgcaggctggacctgctgaggttgaccacagtaatctctatgcattcaatatgagagtcacccagactatcggacgacagctggctcaaataggggacgaaatggacaataaatggcgccaagaaccgcctgtcccatggcagaacctgaatttcgggatttatccttatgtcctaagtaggagagtgttctctggaagaatcctcgctaatctttgggggtctaagattatgccgatattcaggacgtcctggttgcttccacagcttcaaaatggctgtcaggaggctagaaagtgggcagcttgggtgtccaacttgcatgtttctgactggtctcgcagcactacatacaccctggcatctgctttactactggtcactgtgtctatcttccttgtaaactggaatgagtatgaaggctga

SEQ ID NO: 7 - protein, 266 aa, Danio rerio
MTLCFLNTSAALADEEGDPLPTALINSLDLAVNQPVSGSGFCKLKLANEQTVVTLQQLATREPMGDEEEVQGFQSTDPHGTTVCGMARPEMESRVDEHNSGTPNSCRMEVLRQDAWPNGSIIQPCHRRRTIATQTSTLSAPLPHIPSHDAFSLDSVQQQDSLLRDNSGTEQEVSRPLPLPDLLADNQSSSEESTSSSSSTAEEDPTLEEQAVERVAVQLRTIGDEMNAVFLQRNAVPHWQNWRGLYRGLMALVSDTINALYQHGLR

SEQ ID NO: 8 - DNA, 801 bp, Danio rerio
atgactttgtgctttttaaacacaagcgcagcgctcgctgatgaagagggcgatcctctgcccactgctctgataaacagtcttgacctagcagtgaatcagccggtgtcaggttctggcttttgtaaactcaaactggccaatgagcaaactgttgtgactctccaacaattagcaacaagagaacccatgggggatgaagaggaggtgcagggctttcagagcacagacccacacgggacaactgtatgtggaatggcccgaccagagatggaaagcagagtggacgaacataactctggcacgccgaacagctgcaggatggaggtgctgcgtcaggacgcctggccaaatggcagcatcatccagccctgccatcgacgccgaaccattgccactcaaaccagcactctctctgcaccactgccccacatcccctcacatgatgccttcagcttggacagcgtccagcagcaggacagtctactcagggacaattcaggaacagaacaggaagtgtccaggcctcttcctctgccagatctgctagcagacaaccagagctcctcagaggagtccacgtccagcagcagctcgaccgctgaggaggaccccacactggaggagcaggctgtggagagggtggccgtacaactgaggacaattggggacgagatgaacgctgtcttccttcagaggaatgccgtcccgcactggcagaactggagaggcctgtaccgcgggctcatggcgctggtctcggacaccatcaatgccctctaccagcacggcctcagatga

SEQ ID NO: 9 - protein, 185 aa, Danio rerio
MDDEEDEQLPRCCETPLRNKRSEKRDAHGGDVGQTAHRHASTQTAGSVLNSARDADMAPFQGSQREASSLCRVVGARTAFRAPCGTGGLVSLTMGPGARGGPRALFHGNAGFRAHFPALFEPALDGLQNAEQREQDEGRPEEKEEDRDAGISVEVQIGRKLREMGDQFQQEHLQLIILDETRYRI

SEQ ID NO: 10 - DNA, 558 bp, Danio rerio
atggatgatgaggaggatgaacagcttcctcgctgctgtgaaacgccgctaagaaacaagcgctcagagaagagagacgcccacggtggagacgtgggacaaacagctcacagacacgcatccacacagactgctggctctgtcctcaattcagcgagagacgcagatatggcaccattccagggctcacagagagaagcatcatctctttgcagagttgtgggtgctaggactgcattcagggctccctgtggaactgggggccttgtgtcacttactatgggccccggagcccggggtgggccaagagcactttttcatggaaacgctggatttcgtgcacacttccctgcgctgttcgaacccgccctggatggcttacaaaacgccgaacagagagagcaggacgaaggcagaccagaagaaaaagaagaggatcgcgacgcagggattagcgtggaggttcagattggacgtaaattacgtgaaatgggggatcagtttcagcaagagcatcttcagctgattatattagatgaaaccagatacaggatttaa

SEQ ID NO: 11 - protein, 15 aa, Homo sapiens
MEDCLAHLGEKVSQE

SEQ ID NO: 12 - protein, 15 aa, Danio rerio
IEDSLAVLGDRVSRD

SEQ ID NO: 13 - protein, 15 aa, Danio rerio
FDLELKALVQDVNEC

SEQ ID NO: 14 - protein, 15 aa, Homo sapiens
IPECIKQVDQELNGK

SEQ ID NO: 15 - protein, 15 aa, Homo sapiens
IVELLKYSGDQLERK

SEQ ID NO: 16 - protein, 15 aa, Homo sapiens
ALETLRRVGDGVQRN

SEQ ID NO: 17 - protein, 15 aa, Danio rerio
VLSTMRRVVDNLAVK

SEQ ID NO: 18 - protein, 15 aa, Danio rerio
AIPTMKRVVDNILVK

SEQ ID NO: 19 - protein, 15 aa, Homo sapiens
IARHLAQVGDSMDRS

SEQ ID NO: 20 - protein, 15 aa, Danio rerio
IGRQLAQIGDEMDNK

SEQ ID NO: 21 - protein, 15 aa, Homo sapiens
LALRLACIGDEMDVS

SEQ ID NO: 22 - protein, 15 aa, Homo sapiens
LSECLKRIGDELDSN

SEQ ID NO: 23 - protein, 15 aa, Danio rerio
LAQCLQQIGDELDGN

SEQ ID NO: 24 - protein, 15 aa, Danio rerio
LANTIKVIGDKLDQD

SEQ ID NO: 25 - protein, 15 aa, Homo sapiens
IARKLQCIADQFHRL

SEQ ID NO: 26 - protein, 15 aa, Danio rerio
IGQKLQLIGDQFYQE

SEQ ID NO: 27 - protein, 15 aa, Homo sapiens
VGRQLAIIGDDINRR

SEQ ID NO: 28 - protein, 15 aa, Homo sapiens
VHLTLRQAGDDFSRR

SEQ ID NO: 29 - protein, 15 aa, Homo sapiens
IAQELRRIGDEFNAY

SEQ ID NO: 30 - protein, 15 aa, Danio rerio
VARELRRIGDEFNRL

SEQ ID NO: 31 - protein, 15 aa, Danio rerio
VAVQLRTIGDEMNAV

SEQ ID NO: 32 - protein, 15 aa, Danio rerio
CAQQLRNIGDLLNWK

SEQ ID NO: 33 - protein, 15 aa, Homo sapiens
YGRELRRMSDEFVDS

SEQ ID NO: 34 - protein, 15 aa, Danio rerio
YGQQLRRMSDEFDKG

SEQ ID NO: 35 - protein, 15 aa, Homo sapiens
IGAQLRRMADDLNAQ

SEQ ID NO: 36 - protein, 15 aa, Homo sapiens
VKQALREAGDEFELR

SEQ ID NO: 37 - protein, 15 aa, Homo sapiens
LHQAMRAAGDEFETR

SEQ ID NO: 38 - protein, 15 aa, Danio rerio
VKEALRDSANEFELR

SEQ ID NO: 39 - protein, 15 aa, Danio rerio
LYRVLRDAGDEIERI

SEQ ID NO: 40 - protein, 15 aa, Homo sapiens
TAARLKALGDELHQR

SEQ ID NO: 41 - protein, 15 aa, Homo sapiens
VCAVLLRLGDELEMI

SEQ ID NO: 42 - protein, 15 aa, Danio rerio
VSVVLLKLGDELECM

SEQ ID NO: 43 - protein, 15 aa, Danio rerio
MAAELIRIADLLEQS

SEQ ID NO: 44 - protein, 15 aa, Homo sapiens
TKESLAQTSSTITES

SEQ ID NO: 45 - protein, 15 aa, Danio rerio
EAEAVARISDWSSRP

SEQ ID NO: 46 - protein, 15 aa, Homo sapiens
LALRLACIGDEMDVS

SEQ ID NO: 47 - protein, 15 aa, Mus musculus
VALRLACIGDEMDLC

SEQ ID NO: 48 - protein, 15 aa, Danio rerio
IGRQLAQIGDEMDNK

SEQ ID NO: 49 - protein, 15 aa, Homo sapiens
IARHLAQVGDSMDRS

SEQ ID NO: 50 - protein, 15 aa, Mus musculus
IARHLAQIGDEMDHN

SEQ ID NO: 51 - protein, 15 aa, Danio rerio
MAAELIRIADLLEQS

SEQ ID NO: 52 - protein, 15 aa, Homo sapiens
IARKLQCIADQFHRL

SEQ ID NO: 53 - protein, 15 aa, Mus musculus
IARKLQCIADQFHRL

SEQ ID NO: 54 - protein, 15 aa, Danio rerio
IGQKLQLIGDQFYQE

SEQ ID NO: 55 - protein, 15 aa, Danio rerio
IGRKLREMGDQFQQE

SEQ ID NO: 56 - protein, 15 aa, Homo sapiens
YGRELRRMSDEFVDS

SEQ ID NO: 57 - protein, 15 aa, Mus musculus
YGRELRRMSDEFEGS

SEQ ID NO: 58 - protein, 15 aa, Danio rerio
YGQQLRRMSDEFDKG

SEQ ID NO: 59 - protein, 15 aa, Homo sapiens
CATQLRRFGDKLNFR

SEQ ID NO: 60 - protein, 15 aa, Mus musculus
ECAQLRRIGDKVNLR

SEQ ID NO: 61 - protein, 15 aa, Danio rerio
CAQQLRNIGDLLNWK

SEQ ID NO: 62 - protein, 15 aa, Homo sapiens
IGAQLRRMADDLNAQ

SEQ ID NO: 63 - protein, 15 aa, Mus musculus
IGAQLRRMADDLNAQ

SEQ ID NO: 64 - protein, 15 aa, Danio rerio
VAVQLRTIGDEMNAV

SEQ ID NO: 65 - DNA, 24 bp, Danio rerio
gggggttgttgtaaagtacagtgg

SEQ ID NO: 66 - DNA, 25 bp, Danio rerio
tcaggttttctgaaatctcgagacg

SEQ ID NO: 67 - DNA, 27 bp, Danio rerio
actgtactgagacacatcacagcaaca

SEQ ID NO: 68 - DNA, 22 bp, Danio rerio
tcagccttcatactcattccag

SEQ ID NO: 69 - DNA, 35 bp, Danio rerio
ctaactgagtactcatctaatgaattaacaccgct

SEQ ID NO: 70 - DNA, 24 bp, Danio rerio
tcatctgaggccgtgctggtagag

SEQ ID NO: 71 - DNA, 21 bp, Danio rerio
atggatgaggacgaggatgat

SEQ ID NO: 72 - DNA, 19 bp, Danio rerio
tcacctgcggttctctctg

SEQ ID NO: 73 - DNA, 22 bp, Danio rerio
atggatgatgaggaggatgaac

SEQ ID NO: 74 - DNA, 26 bp, Danio rerio
ttaaatcctgtatctggtttcatcta

SEQ ID NO: 75 - DNA, 25 bp, Danio rerio
cttccacaacacttccactggatac

SEQ ID NO: 76 - DNA, 25 bp, Danio rerio
ttcactgtcagtcactctgcggggc

SEQ ID NO: 77 - DNA, 37 bp, Danio rerio
gccgaattccaccatgtcttactataaccgagaactg

SEQ ID NO: 78 - DNA, 36 bp, Danio rerio
ggcctcgagtcacaggcgtttctgtgcaatgagtcc

SEQ ID NO: 79 - DNA, 18 bp, Danio rerio
atggctaacgaaattagc

SEQ ID NO: 80 - DNA, 21 bp, Danio rerio
tcacttctgagcaaaaaaggc

SEQ ID NO: 81 - DNA, 19 bp, Danio rerio
atggagatgttgcgccgct

SEQ ID NO: 82 - DNA, 27 bp, Danio rerio
tcatttctctctcagcaggtagaaaac

SEQ ID NO: 83 - DNA, 19 bp, Danio rerio
atgaacgtgttcgcgcgct

SEQ ID NO: 84 - DNA, 23 bp, Danio rerio
gcgcaagtctacttcgtcaggta

SEQ ID NO: 85 - DNA, 19 bp, Danio rerio
aaggggctgcagctggacg

SEQ ID NO: 86 - DNA, 22 bp, Danio rerio
agcggggctcaggtcagtacag

SEQ ID NO: 87 - DNA, 20 bp, Danio rerio
catcttcgattgatttgatt

SEQ ID NO: 88 - DNA, 22 bp, Danio rerio
atagtttgaaagttctgaatcc

SEQ ID NO: 89 - DNA, 19 bp, Danio rerio
atgtcctgttggttgaggg

SEQ ID NO: 90 - DNA, 21 bp, Danio rerio
tcagcgtactaagaggaaggt

SEQ ID NO: 91 - DNA, 25 bp, Danio rerio
gcctaaaatccaaactcagagccat

SEQ ID NO: 92 - DNA, 25 bp, Danio rerio
tgtcgttgtttcttccagcgaacat

SEQ ID NO: 93 - DNA, 25 bp, Danio rerio
aggttgttgctcgttctccgatgtc

SEQ ID NO: 94 - DNA, 25 bp, Danio rerio
gtcatagctaatttcgttagccatg

SEQ ID NO: 95 - DNA, 25 bp, Danio rerio
tgaaaataagcgaactgaagaagac

SEQ ID NO: 96 - DNA, 25 bp, Danio rerio
atttttcggctaaaacgtgtatggg

SEQ ID NO: 97 - DNA, 25 bp, Danio rerio
ggtcaaagttcctgttgaagtccat

SEQ ID NO: 98 - DNA, 25 bp, Danio rerio
acacatcatcctcgtcctcatccat

SEQ ID NO: 99 - DNA, 25 bp, Danio rerio
ctttcttcgccatttcagcaagttt

SEQ ID NO: 100 - DNA, 25 bp, Danio rerio
tgctttccatctctggtcgggccat

SEQ ID NO: 101 - DNA, 25 bp, Danio rerio
ctacaaacaaggacacaatggtgga

SEQ ID NO: 102 - DNA, 23 bp, Danio rerio
gcgccattgctttgcaagaattg

SEQ ID NO: 103 - DNA, 25 bp, Danio rerio
cctcttacctcagttacaatttata

SEQ ID NO: 104 - DNA, 21 bp, Danio rerio
tgcgttcgtctctgtagatgt

SEQ ID NO: 105 - DNA, 19 bp, Danio rerio
gcctgtggagtgacactga

SEQ ID NO: 106 - DNA, 35 bp, Danio rerio
tgtgtgtgtgtgttagtttcttttgacagtatttg

SEQ ID NO: 107 - DNA, 21 bp, Danio rerio
cgaacctgtgacagaaacttg

SEQ ID NO: 108 - DNA, 19 bp, Danio rerio
ctgcgcgcactctactaca

SEQ ID NO: 109 - DNA, 25 bp, Danio rerio
cggtttgctctttcttcgccatttc

SEQ ID NO: 110 - DNA, 22 bp, Danio rerio
gaacacacgggttacaaaagac

SEQ ID NO: 111 - DNA, 24 bp, Danio rerio
gaaaattcccagagtctgtaagtg

SEQ ID NO: 112 - DNA, 21 bp, Danio rerio
acgagtgcaggcgctctcctt

SEQ ID NO: 113 - DNA, 27 bp, Danio rerio
accatgatggtcccggcgaacagccgc

SEQ ID NO: 114 - DNA, 27 bp, Danio rerio
actttacagatccaatcggaaagctcc

SEQ ID NO: 115 - DNA, 24 bp, Danio rerio
atcatggtgcagcctaaaaatcgt

SEQ ID NO: 116 - DNA, 24 bp, Danio rerio
ccctcagaggtcaaacaggaaggc

SEQ ID NO: 117 - DNA, 24 bp, Danio rerio
cacgcgatggtcagcatgacaagc

SEQ ID NO: 118 - DNA, 24 bp, Danio rerio
tactgactagctcaccagaaatgc

SEQ ID NO: 119 - DNA, 27 bp, Danio rerio
accatgacatccaaccttcctatcggt

SEQ ID NO: 120 - DNA, 27 bp, Danio rerio
agtttatttaatcatgaatgccccaaa

SEQ ID NO: 121 - DNA, 24 bp, Danio rerio
atgagtgctaacttcggccactcg

SEQ ID NO: 122 - DNA, 24 bp, Danio rerio
tcagtggatcttaaagaggccgaa

SEQ ID NO: 123 - DNA, 30 bp, Danio rerio
gcaaccatgaagcttgagagtcgggcgttt

SEQ ID NO: 124 - DNA, 30 bp, Danio rerio
tttcgttcacaaaccaaacaccccaaagaa

SEQ ID NO: 125 - DNA, 24 bp, Danio rerio
atggtgagatacgaaacaacatta

SEQ ID NO: 126 - DNA, 30 bp, Danio rerio
attaaatcacaacgcgaacaccccgaagaa